(19) United States
(12) Patent Application Publication
(10) Pub. No.: US 2003/0175760 A1
(43) Pub. Date: Sep. 18, 2003

(54) Discovery of therapeutic products

(76) Inventors: Wynn L. Walker, Palo Alto, CA (US); John S. Babcook, Vancouver (CA); C. Geoffrey Davis, Burlingame, CA (US); Larry L. Green, San Francisco, CA (US); Jaspal Singh Kang, Surrey (CA); Xiao-Chi Jia, San Mateo, CA (US); Michael L. Gallo, North Vancouver (CA); Keith Joho, San Jose, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/309,418

(22) Filed: Dec. 2, 2002

Related U.S. Application Data
(60) Provisional application No. 60/337,278, filed on Dec. 3, 2001.

(51) Int. Cl. C12Q 1/68; G01N 33/53

(52) U.S. Cl. 435/6; 435/7.1

(57) ABSTRACT

Methods to screen antibodies against an antigen, categorize them according to the epitope they recognize, and rank them according to their binding affinities, thereby providing a method to rapidly and efficiently identify antibodies having potential usefulness in therapeutic products are described. Also described are methods of evaluating antibodies to determine their potential usefulness in therapeutic products.

### RELATED APPLICATIONS

This application claims priority to provisional U.S. Patent Application Serial No. 60/337278, filed Dec. 3, 2001.

### FIELD OF THE INVENTION

The present invention relates to discovery of therapeutic products. The present invention provides methods to screen, categorize, and rank antibodies based on their epitope recognition properties and binding affinities, in order to identify antibodies with potential usefulness in therapeutic products. Further provided are methods of evaluating antibodies that have been screened, categorized, and ranked according the methods of the invention, to determine their potential usefulness in therapeutic products.

### BACKGROUND OF THE INVENTION

Antibodies are regarded as an important resource for developing effective therapeutic products because of their combination of variability and specificity, i.e., antibodies can be elicited against a wide variety of target antigens and antibodies recognize a single epitope on the target antigen. This specificity is best used against a target antigen that appears to be limited to a specific disease condition, such as a surface antigen found only on cancer cells, or a surface antigen specific to a disease-causing organism. Antibodies are of particular interest for the development of anticancer agents, where a key to the development of successful anticancer agents is the ability to design agents that will selectively kill cancer cells while exerting relatively little, if any, untoward effects against normal tissues. To this end, much research has focused on identifying cancer-cell-specific marker antigens that can serve as immunological targets both for chemotherapy and diagnosis.

Antibodies can function in therapeutic products through various mechanisms. In the simplest model, antibody binding to a target antigen on the surface of a cell triggers destruction, malfunctioning, or neutralization of the cell. Antibody binding may trigger cell destruction through apoptosis, necrosis, or by eliciting other cells such as macrophages to destroy and remove the cell, in particular a cancer cell. Antibodies may cause malfunctioning of a diseased cell, in particular a cancer cell, by interfering with normal processes. For example, antibodies may bind to and inhibit receptors or kinases which are expressed only in cancer cells, or which are overexpressed in cancer cells. Antibodies may also have a neutralizing effect in which they bind to toxic antigens, viral antigens, or antigens involved in various essential cell processes such as transcription or signal transduction, and block the action of these antigens. Therapeutic antibodies may induce effector mechanisms such as antibody-dependent cellular cytotoxicity (ADCC) and complement-dependent cytolysis.

In a different model, antibodies are conjugated to a cytotoxin to produce a therapeutic product known as an immunotoxin. This approach utilizes the specificity and affinity of antibodies to deliver cytotoxic agents to a target cell in an approach sometimes known as the “magic bullet”. Antibodies, typically a tumor-directed antibody or antibody fragment, are conjugated with a cytotoxic agent or toxic moiety active against the target cell. The antibody acts as a targeting agent to find and bind to a cell bearing the target antigen, thereby delivering the toxin which selectively kills the cell carrying the target antigen. Recently, stable and long-lived immunotoxins have been developed for the treatment of a variety of malignant diseases by preventing unwanted reactions. For example, deglycosylated ricin A chain appears to prevent entrapment of the immunotoxin by the liver and hepatotoxicity. If necessary, crosslinkers can be chosen which endow immunotoxins with high in vivo stability.

Antibodies as therapeutic products are described, e.g., in U.S. Pat. No. 6,319,500 disclosing an immunotoxin (immunoconjugate) comprising an antibody coupled to a therapeutic agent, in U.S. Pat. No. 6,319,499 disclosing the use of an antibody or antibody fragment to activate a receptor, in U.S. Pat. No. 6,316,462 disclosing an antibody directed the extracellular domain of a growth factor receptor; in U.S. Pat. No. 6,312,691 disclosing an antibody that activates a tumor-specific member of the tumor necrosis factor receptor family, and U.S. Pat. No 6,294,173 disclosing an immunotoxin targeted against fibrin in tumors.

Immunotoxins have proven highly effective at treating lymphomas and leukemias in mice and in humans. Lymphoid neoplasias are particularly amenable to immunotoxin therapy because the tumor cells are relatively accessible to blood-borne immunotoxins. In addition, an immunotoxin comprising a monoclonal antibody conjugated to granulocyte-macrophage colony-stimulating factor (GM-CSF) induced complete remission of bone marrow (BM) disease in many neuroblastoma patients. Kushner et al., 2001, J Clin Oncol 19:4189-4194. In contrast, immunotoxins have proved relatively ineffective against solid tumors such as carcinomas. Reasons for this are that solid tumors are generally impermeable to antibody-sized molecules, antibodies that enter the tumor mass do not distribute evenly due to a physical barrier of tumor cells and fibrous tumor stromas, the distribution of blood vessels in most tumors is disorganized and heterogeneous, and all the antibody entering a tumor may become adsorbed in perivascular regions by the first tumor cells encountered, leaving none to reach tumor cells at more distant sites.

Nonetheless, antibody-based therapeutic products continue to be tested and released, with monoclonal antibodies being of greatest interest. Monoclonal antibodies that have been introduced into human include: OKT3, which binds to a molecule on the surface of T cells and is used to prevent acute rejection of organs; LymphoCide, which binds to CD22, a molecule found on some B-cell leukemias; Rituximab (trade name, Rituxan) which binds to the CD20 molecule found on most B-cells and is used to treat B-cell lymphomas; Lym-1 (trade name, Oncolym), which binds to the HLA-DR-encoded histocompatibility antigen that can be expressed at high levels on lymphoma cells; Daclizumab (trade name, Zenopax), which binds to part of the IL-2 receptor produced at the surface of activated T cells and is used to prevent acute rejection of transplanted kidneys; Infliximab, which binds to tumor necrosis factor-alpha (TNF-alpha) and shows promise against some inflammatory diseases such as rheumatoid arthritis; Herceptin, which binds HER-2/neu, a growth factor receptor found on some tumor cells, including some breast cancers and lymphomas, and has the distinction of being first therapeutic monoclonal antibody that appears to be effective against solid tumors; Vitaxin, which binds to a vascular integrin (anb3) found on the blood vessels of tumors but not on the blood vessels supplying normal tissues; and Abciximab (trade name, Reopro), which inhibits the clumping of platelets by binding the receptors on their surface that normally are linked by fibrinogen. The immunotoxin compound CMA-676 is a conjugate of a monoclonal antibody that binds CD33, a cell-surface molecule expressed by the cancerous cells in acute myelogenous leukemia (AML), and calicheamicin, an oligosaccharide that blocks the binding of transcription factors to DNA and thereby inhibiting transcription in AML cancer cells.

The large number of target antigens that may serve as markers or effectors of disease creates a need for a rapid, efficient, and effective method for identifying antibodies with potential as therapeutic products directed against these antigens. However, the large numbers of antibodies generated against a particular target antigen may vary substantially in terms of both how strongly they bind to the antigen as well as the particular epitope they bind to on the target antigen. In order to identify therapeutically useful antibodies from the large number of generated candidate antibodies, it is necessary to screen large numbers of antibodies for their binding affinities and epitope recognition properties. For this reason, it would be advantageous to have a rapid method of screening antibodies generated against a particular target antigen to identify those antibodies that are most likely to have a therapeutic effect. In addition, it would be advantageous to provide a mechanism of categorizing the generated antibodies according to their target epitope binding sites.

### SUMMARY OF THE INVENTION

The present disclosure provides methods to screen, categorize, and rank antibodies based on their epitope recognition properties and binding affinities, and methods of evaluating antibodies that have been screened, categorized, and ranked according the methods of the invention, to determine their potential usefulness in or as therapeutic products. One embodiment of the present invention is a method of concurrently (i) determining the potential therapeutic utility of a protein target in connection with a molecule that interacts with such protein target and (ii) identifying molecules that interact with such protein target that enable such therapeutic utilities. In the method, a protein target is screened against a plurality of molecules to find which of those molecules interact. The interactive molecules are categorized according to predefined criteria and representative members are selected for use in preselected assays with the protein target. Activities identified in the assays are logged and analyzed and positive activities in the assays are indicative of the potential therapeutic utility of the protein target and the interactive molecules that enable such utility are identified.

As will be appreciated, interactive molecules may include small molecules, proteins, peptides, antibodies, and the like. In a preferred embodiment, the interactive molecules are antibodies and preferably human antibodies. The target protein may be a known protein of generally known function or utility. Or, the target protein may be novel and of relatively unknown function. In connection with the categorization of the interactive molecules, in general, it is preferred that different binding sites on the antigen target are represented and that binding affinity to the target is optimized. Assays are selected based upon the therapeutic utility that is being considered. For example, assays related to oncology, inflammation, or the like may be utilized as the case may be.

One embodiment of the present invention is a method to screen antibodies against an antigen, categorize them according to the epitope they recognize, and rank them according to their binding affinities, thereby providing a method to rapidly and efficiently identify antibodies having potential usefulness in therapeutic products. Further provided are methods of evaluating antibodies to determine their potential usefulness in therapeutic products.

Another embodiment of the invention is a method utilizing epitope binning to screen, categorize, or “bin” antibodies according to the epitope they recognize, and then rank the antibodies within each category or “bin” according to their affinity for an epitope, using a limiting antigen dilution assay for binding affinity. This method is preferably used to screen a panel of antibodies generated against an antigen, using a competitive binding assay to discern the epitope recognition properties of the panel, then using a clustering process to bin the antibodies in the panel, and then using a limiting antigen dilution assay to kinetically rank the antibodies in the panel based on their binding affinity.

Yet another embodiment of the invention is a method to determine the therapeutic potential of any antibody identified by epitope binning and limiting antigen dilution as being a high-affinity antibody against an antigen of interest. The antibody may be evaluated for its ability act directly on cells to bring out the desired effect and/or it may be evaluated for its suitability for use in a conjugated form such as an immunotoxin. The antibody may be evaluated for its potential usefulness in a therapeutic product to treat a disorder or disease state in a mammal, preferably a human, or it may be evaluated for its potential usefulness in a therapeutic product to enhance cell function or confer a beneficial effect on a mammal, preferably a human.

Embodiments of the invention provide methods for screening, categorizing, and ranking a heterogeneous panel of antibodies raised against different epitopes on an antigen, providing to method to identify which epitopes are better targets for therapeutic products directed against a particular antigen

In addition, embodiments of the invention provide methods for screening, categorizing, and ranking conjugated antibodies, to determine their potential usefulness in therapeutic products.

Also, the methods described herein may be used to evaluate antibodies against disease-specific antigens, preferably antibodies directed against cancer antigens, in particular antigens associated with solid tumors, to evaluate their potential usefulness in anti-neoplastic therapeutic products.

### BRIEF DESCRIPTION OF THE DRAWINGS

FIG. 1. Schematic illustration of one embodiment of an epitope binning assay using labelled bead technology in a single well of a microtiter plate. As illustrated here, each reference antibody is coupled to a bead with distinct emission spectrum, where the reference antibody is coupled through a mouse anti-human monoclonal capture antibody, forming a uniquely labelled reference antibody. The entire set of uniquely labelled reference antibodies is placed in the well of a multiwell microtiter plate. The set of reference antibodies are incubated with antigen, and then a probe antibody is added to the well. A probe antibody will only bind to antigen that is bound to a reference antibody that recognizes a different epitope. Binding of a probe antibody to antigen will form a complex consisting of a reference antibody coupled to a bead through a capture antibody, the antigen, and the bound probe antibody. A labelled detection antibody is added to detect bound probe antibody. Here, the detection antibody is labelled with biotin, and bound probe antibody is detected by the interaction of streptavidin-PE and the biotinylated detection antibody. As shown in FIG. 1, Antibody #50 is used as the probe antibody, and the reference antibodies are Antibody #50 and Antibody #1. Probe Antibody #50 will bind to antigen that is bound to reference Antibody #1 because the antibodies bind to different epitopes, and a labelled complex can be detected. Probe antibody #50 will not bind to antigen that is bound by reference antibody #50 because both antibodies are competing for the same epitope, such that no labelled complex is formed.

FIG. 2. Correlation between blocking buffer intensity values and average intensity.

FIG. 2A. Correlation between blocking buffer intensity and average intensity within rows. Blocking buffer intensity value for each row (y-axis) plotted against the average intensity value of the row with blocking buffer value omitted (x-axis). Fitting a line to the data shows a strong linear correlation between the blocking buffer values and the average intensity values of the rest of the row.

FIG. 2B. Correlation between blocking buffer intensity and average intensity within columns. Blocking buffer intensity value for each column (y-axis) plotted against the average intensity value of the column with blocking buffer value omitted (x-axis). Fitting a line to the data shows a relatively weak linear correlation between the blocking buffer values and the average intensity values of the rest of the column.

FIG. 2C. Scatter plot of intensity values for the matrix with antigen and background-normalized matrix. this plot shows a tight linear correlation (slope about 1.0) for high subtracted signal values, indicating that the background signal is minimal relative to the signal in the presence of antigen. The points are shaded according to the value of the fraction, calculated as the subtracted signal divided by the signal for the experiment with antigen present. Smaller fraction values (closer to zero) correspond to high background contribution and have light shading. Larger fraction values (closer to 1) correspond to lower background contribution and have darker shading. The distribution of the smaller fraction values predominantly in the lower-left region of the scatter plot suggests that the contribution of background becomes less for subtracted signal values greater than 1000.

FIG. 3. Comparison of epitope binning results with FACS results. Results from antibody experiments using the ANTIGEN39 antibody are shown, comparing results using the epitope binning method described herein with results using flow cytometry (fluorescence-activated cell sorter, FACS). Antibodies are assigned to bins 1-15, as indicated by rows 1-15 in the far left column using the epitope binning assay. Shading in cells indicates antibodies that are FACS positive for cells expressing ANTIGEN39 (cell line 786-0), and no shading indicates antibodies that are negative for cells that do not express ANTIGEN39 (cell line M14).

FIG. 4. Dissimilarity vs. background value: effect of choice of threshold cutoff value. The figure shows the amount of dissimilarity between antibodies 2.1 and 2.25 calculated at various threshold values. The amount of dissimilarity represents the value for the dissimilarity matrix for the entry corresponding to the two antibodies, Ab 2.1 and Ab 2.25 for a series of dissimilarity matrices computed using different threshold values. Here, the x-axis is the threshold value, and the y-axis is the dissimilarity value calculated using that threshold cutoff value.

FIG. 5. Dendrogram for the ANTIGEN14 antibodies. The length of branches connecting two antibodies is proportional to the degree of similarity between the two antibodies. This figure shows that there are two very distinct epitopes recognized by these antibodies. One epitope is recognized by antibodies 2.73, 2.4, 2.16, 2.15, 2.69, 2.19, 2.45, 2.1, and 2.25. A different epitope is recognized by antibodies 2.13, 2.78, 2.24, 2.7, 2.76, 2.61, 2.12, 2.55, 2.31, 2.56, and 2.39. Antibody 2.42 does not have a pattern that is very similar to any other antibody, but has some noticeable similarity to the second cluster, although it may recognize yet a third epitope which partially overlaps with the second epitope.

FIG. 6. Dendrograms for ANTIGEN39 antibodies.

FIG. 6A. Dendrogram for the ANTIGEN39 antibodies for five input experimental data sets. The number o unique clusters of antibodies suggests that are several different epitopes, some of which may overlap. For example, the cluster containing antibodies 1.17, 1.55, 1.16, 1.11 and 1.12 and the cluster containing 1.21, 2.12, 2.38, 2.35, and 2.1 appear to be fairly closely related, with each antibody pair with the exception of 2.35 and 1.11 being no more than 25% different. This high degree of similarity across the two clusters suggests that the two different epitopes themselves have a high degree of similarity.

FIG. 6B. Dendrogram for the ANTIGEN39 antibodies for Experiment 1. Antibodies 1.12, 1.63, 1.17, 1.55, and 2.12 consistently cluster together in this experiment as well as in other experiments, as do antibodies 1.46, 1.31, 2.17, and 1.29.

FIG. 6C. Dendrogram for 5 the ANTIGEN39 antibodies for Experiment 2. Antibodies 1.57 and 1.61 consistently cluster together in this experiment as well as in other experiments.

FIG. 6D. Dendrogram for the ANTIGEN39 antibodies for Experiment 3. Antibodies 1.55, 1.12, 1.17, 2.12, 1.11 and 1.21 consistently cluster together in this experiment as well as in other experiments.

FIG. 6E. Dendrogram for the ANTIGEN39 antibodies for Experiment 4. Antibodies 1.17, 1.16, 1.55, 1.11 and 1.12 consistently cluster together in this experiment as well as in other experiments, as do antibodies 1.31, 1.46, 1.65, and 1.29, as well as antibodies 1.57 and 1.61.

FIG. 6F. Dendrogram for the ANTIGEN39 antibodies for Experiment 5. Antibodies 1.21, 1.12, 2.12, 2.38, 2.35, and 2.1 consistently cluster together in this experiment as well as in other experiments.

FIG. 7. Dendrograms for clustering IL-8 monoclonal antibodies.

FIG. 7A. Dendrograms for a clustering of seven IL-8 monoclonal antibodies. The dendrogram on the left is generated by clustering columns, and the dendrogram on the right by clustering rows of a background-normalized signal intensity matrix. Both dendrograms indicate that there are two epitopes, using a dissimilarity cutoff of 0.25: one epitope is recognized by monoclonal antibodies HR26, a215, a203, a393, and a452; a second epitope is recognized by monoclonal antibodies K221 and a33.

FIG. 7B. Dendrograms for IL-8 monoclonal antibodies from a combined clustering analysis merging five different experimental data sets. The dendrogram on the left was generated by clustering columns, whereas the dendrogram on the right was generated by clustering rows of the background-normalized signal intensity matrix. Both dendrograms indicate that there are two epitopes, using a dissimilarity cut-off of 0.25: one epitope is recognized by monoclonal antibodies a809, a928, HR26, a215, and D111; a second epitope is recognized by monoclonal antibodies a837, K221, a33, a142, a358, and a203, a393, and a452.

FIG. 7C. Dendrograms for a clustering of nine IL-8 monoclonal antibodies. The dendrogram on the left was generated by clustering columns, and the dendrograms on the right by clustering rows of the background-normalized signal intensity matrix. Both dendrograms indicate that there are two epitopes, using a dissimilarity cut-off of 0.25: one epitope is recognized by monoclonal antibodies HR26 and a215; a second epitope is recognized by monoclonal antibodies K221, a33, al42, a203, a358, a393, and a452.

FIG. 8. Intensity matrices generated in the embodiment disclosed in Example 2 using a set of antibodies against ANTIGEN14.

FIG. 8A is a table showing the intensity matrix for experiment conducted with antigen.

FIG. 8B is a table showing the intensity matrix for the same experiment conducted without antigen (control). These matrices are used a input data matrices for subsequence steps in data analysis.

FIG. 9. Difference matrix for antibodies against the ANTIGEN14 target. Difference matrix is generated by subtracting the matrix corresponding to values obtained from experiment without antigen (see FIG. 8B) from the matrix corresponding to values obtained from the experiment with antigen (see FIG. 8A) disclosed in Example 2.

FIG. 10. Adjusted difference matrix with minimum threshold value. For the intensity values of Example 2, the minimum reliable signal intensity value is set to 200 intensity units and values below the minimum threshold are set to the threshold of 200.

FIG. 11. Row normalized matrix. Each row in the adjusted difference matrix of FIG. 10 is adjusted by dividing it by the last intensity value in the row, which corresponds to the intensity value for beads to which blocking buffer is added in place of primary antibody. This adjusts for well-to-well intensity.

FIG. 12. Diagonal normalized matrix. All columns except the one corresponding to Antibody 2.42 were column-normalized. Dividing each column by its corresponding diagonal is carried out to measure each intensity relative to an intensity that is known to reflect competition—i.e., competition against self.

FIG. 13. Antibody pattern recognition matrix. For data from the embodiment disclosed in Example 2, intensity values below the user-defined threshold were set to zero. The user-defined threshold was set to two (2) times the diagonal intensity values. Remaining values were set to one.

FIG. 14. Dissimilarity matrix. For data from the embodiment disclosed in Example 2, a dissimilarity matrix is generated from the matrix of zeroes and ones shown in FIG. 13, by setting the entry in row i and column j to the fraction of the positions at which two rows, i and j, differ. FIG. 14 shows the number of positions, out of 22 total, at which the patterns for any two antibodies differed for set of antibodies generated against the ANTIGEN14 target.

FIG. 15. Average dissimilarity matrix. After separate dissimilarity matrices were generated from each of several threshold values ranging from 1.5 to 2.5 times the values of the diagonals, the average of these dissimilarity matrices was computed (FIG. 15) and used as input to the clustering process.

FIG. 16. Permuted average dissimilarity matrix. For data from the embodiment disclosed in Example 2, clusters can be visualized in matrices. In FIG. 16, the rows and columns of the dissimilarity matrix were rearranged according to the order of the “leaves ” or leaves on the dendrogram shown in FIG. 5, and individual cells were visually coded according to the degree of dissimilarity.

FIG. 17. Permuted normalized intensity matrix. For data from the embodiment disclosed in Example 2, rows and columns of the normalized intensity matrix were rearranged according to the order of the leaves on the dendrogram shown in FIG. 5, and individual cells were visually coded according to their normalized intensity values.

FIG. 18. Permuted average dissimilarity matrix for five ANTIGEN39 input data sets. Data from five experiments that were conducted using antibodies against the ANTIGEN39 target (see Example 3) produced five input data sets. Dissimilarity matrices were generated for each input data set, and an average dissimilarity matrix was generated, and rows and columns were arranged (permuted) according to arrangement of the corresponding dendrogram(s) shown in FIG. 6.

FIG. 19. Permuted normalized intensity matrix for five ANTIGEN39 input data sets. Data from five experiments that were conducted using antibodies against the ANTIGEN39 target (see Example 3) produced five input data sets. A normalized intensity matrix was generated for the five input data sets and rows and columns were arranged (permuted) according to arrangement of the corresponding dendrogram(s) shown in FIG. 6.

FIG. 20. Permuted average dissimilarity matrix for Experiment 1 using a set of antibodies against the ANTIGEN39 target. Data from the set of antibodies analyzed in Experiment 1 (Example 3) were analyzed. See dendrogram shown in FIG. 6B.

FIG. 21. Permuted normalized intensity matrix for Experiment 1 using a set of antibodies against the ANTIGEN39 target. Data from the set of antibodies analyzed in Experiment 1 (Example 3) were analyzed. See dendrogram shown in FIG. 6B.

FIG. 22. Permuted average dissimilarity matrix for Experiment 2 using a set of antibodies against the ANTIGEN39 target. Data from the set of antibodies analyzed in Experiment 2 (Example 3) were analyzed. See dendrogram shown in FIG. 6C.

FIG. 23. Permuted normalized intensity matrix for Experiment 2 using a set of antibodies against the ANTIGEN39 target. Data from the set of antibodies analyzed in Experiment 2 (Example 3) were analyzed. See dendrogram shown in FIG. 6C.

FIG. 24. Permuted average dissimilarity matrix for Experiment 3 using a set of antibodies against the ANTIGEN39 target. Data from the set of antibodies analyzed in Experiment 3 (Example 3) were analyzed. See dendrogram shown in FIG. 6D

FIG. 25. Permuted normalized intensity matrix for Experiment 3 using a set of antibodies against the ANTIGEN39 target. Data from the set of antibodies analyzed in Experiment 3 (Example 3) were analyzed. See dendrogram shown in FIG. 6D.

FIG. 26. Permuted average dissimilarity matrix for Experiment 4 using a set of antibodies against the ANTIGEN39 target. Data from the set of antibodies analyzed in Experiment 4 (Example 3) were analyzed. See dendrogram shown in FIG. 6E.

FIG. 27. Permuted normalized intensity matrix for Experiment 4 using a set of antibodies against the ANTIGEN39 target. Data from the set of antibodies analyzed in Experiment 4 (Example 3) were analyzed. See dendrogram shown in FIG. 6E.

FIG. 28. Permuted average dissimilarity matrix for Experiment 5 using a set of antibodies against the ANTIGEN39 target. Data from the set of antibodies analyzed in Experiment 5 (Example 3) were analyzed. See dendrogram shown in FIG. 6F.

FIG. 29. Permuted normalized intensity matrix for Experiment 5 using a set of antibodies against the ANTIGEN39 target. Data from the set of antibodies analyzed in Experiment 5 (Example 3) were analyzed. See dendrogram shown in FIG. 6F.

FIG. 30. Clusters identified in Experiments 1-5 using sets of antibodies against the ANTIGEN39 target. FIG. 30 summarizes the clusters identified for each of the five individual data sets and for the combined data set for all of the antibodies generated in all five experiments disclosed in Example 3.

### DETAILED DESCRIPTION

Embodiments of the present invention provide methods to discover new therapeutic products and allow validation of the therapeutic potential of intervention with protein targets using interactive molecules, such as antibodies.

In general, one embodiment of the present invention is a method of concurrently (i) determining the potential therapeutic utility of a protein target in connection with a molecule that interacts with such protein target and (ii) identifying molecules that interact with such protein target that enable such therapeutic utilities. In the method, a protein target is screened against a plurality of molecules to find which of those molecules interact. The interactive molecules are categorized according to predefined criteria and representative members are selected for use in pre-selected assays with the protein target. Activities identified in the assays are logged and analyzed and positive activities in the assays are indicative of the potential therapeutic utility of the protein target and the interactive molecules that enable such utility are identified.

As will be appreciated, interactive molecules may include small molecules, proteins, peptides, antibodies, and the like. In a preferred embodiment, the interactive molecules are antibodies and preferably human antibodies. The target protein may be a known protein of generally known function or utility. Or, the target protein may be novel and of relatively unknown function. In connection with the categorization of the interactive molecules, in general, it is preferred that different binding sites on the antigen target are represented and that binding affinity to the target is optimized. Assays are selected based upon the therapeutic utility that is being considered. For example, assays related to oncology, inflammation, or the like may be utilized as the case may be.

As will be appreciated, in the case of a protein target that appears to have homology with certain oncology targets, it is not known whether interaction with the target will result in therapeutic utility. For example, a target may be expressed in normal tissue and interaction with certain interactive molecules could have non-tumor specific effects and, thus, such target would not have beneficial therapeutic utility. On the other hand, even in such case, certain interactive molecules could be determined to provide tumor specific response. In this way, the target would be determined to possess potential therapeutic utility when interactive molecules of determined criteria are utilized. In the process, both the potential therapeutic utility of the protein target and the type and criteria of the interactive molecules are validated.

Relevant assays and screens for activity in oncology, inflammation and the like are well-known to those of skill in the art.

The present invention discloses the discovery discussed above in the context of the utilization and generation of antibodies as the interactive molecules. In a preferred embodiment of the invention in connection with antibodies as the interactive molecules, discovery methods include a combination of epitope binning and limiting antigen dilution assays, which can be used to screen antibodies against a protein target (or antigen), categorize them according to the epitope they recognize, and rank them according to their binding affinities, thereby providing a method to rapidly and efficiently identify antibodies having potential usefulness in therapeutic products. Further provided are methods of evaluating antibodies that have been screened, categorized, and ranked according the methods of the invention, to determine their potential usefulness in therapeutic products.

The present invention provides methods for identifying and evaluating antibodies for use in therapeutic products to treat a disorder or disease state in a mammal, preferably a human. The present invention also provides methods for identifying and evaluating antibodies for use in therapeutic products to enhance target cell function in a mammal, preferably a human. The methods of the present invention may be used to identify and evaluate native antibodies, antibody fragments, chimeric antibodies, monoclonal antibodies, polyclonal antibodies, multispecific antibodies. Preferably, methods of the present invention are practiced using isolated antibodies.

One aspect of the present invention provides a method for screening a panel of antibodies using epitope binning to categorize or “bin” the antibodies according to the epitope they recognize. In conjunction with binning, the antibodies within each category or “bin” are ranked according to their affinity for an epitope, using a limiting antigen dilution assay for binding affinity. In one embodiment, a panel of antibodies may be screened using a competitive binding assay to discern the epitope recognition properties of the panel, then sorted using a clustering process to bin the antibodies in the panel, and then kinetically ranked using a limiting antigen dilution assay to determine the binding affinity of the antibodies in the panel.

Another aspect of the invention provides methods to determine the therapeutic potential of any antibody identified by epitope binning and limiting antigen dilution as being a high-affinity antibody against an antigen of interest. The antibody may be evaluated for its ability act directly on cells to bring out the desired effect and/or it may be evaluated for its suitability for use a conjuated form such as an immunotoxin.

Antibodies identified by epitope binning and limiting antigen dilution as being high-affinity antibodies against an antigen of interest may be evaluated for characteristics such as the ability to have a direct effect on a target cell. Such antibodies may be tested for ability fix complement and elicit complement-dependent cytolysis, or their ability to elicit antibody-dependent cellular cytotoxicity (ADCC). Antibodies can also be tested for their action directly on target cells, for example by inducing apoptosis (programmed cell death) or inhibition of cell metabolism, including proliferation.

Antibodies may also be evaluated for their ability to work synergistically with the host's immune effector mechanisms, for example to enhance antibody-dependent cellular cytotoxicity (ADCC) and complement-dependent cytolysis. Antibodies that bind effectors such as the extracellular domains of receptors involved in a disease process may be tested for the ability to directly activate the receptor and/or block ligand binding to receptors. (Here, ligands may be agonists, antagonists, or small molecules that affect receptor activity.) The antibody may be tested for its ability to act as a neutralizing antibody by neutralizing antigens or exercising neutralizing effects on essential cellular processes involved in the disease state.

A further aspect of the present invention provides methods to determine the immunotoxin suitability of any antibody identified by epitope binning and limiting antigen dilution as a high-affinity antibody against an antigen associated with a disease condition. These antibodies may be useful therapeutic products when conjugated to a cytotoxin to form an immunotoxin, wherein the antibody can deliver the cytotoxin to a defined antigen on a target cell with great precision and high affinity, and the cytotoxin can effect inhibition or destruction of the target cell. As part of an immunotoxin, the antibody may act as a potentiator, targeting compound, carrier, and/or delivery agent for the cytotoxin to which the antibody is conjugated.

High-affinity antibodies against disease-associated antigens such as differentiation markers, growth factors receptors, surface markers of tumor vasculature, disease-specific carbohydrate molecules including glycolipids and glycoproteins, viral surface proteins, or surface immunoglobins, may be conjugated with cytotoxins to form an immunotoxin, and the ability of the immunotoxin to selectively kill target cells may be tested. Antibodies that bind to possible effectors such as receptors, ion channels, or other transmembrane proteins may be evaluated for their ability to deliver an agent that selectively disables the effector. Antibodies may also be used to test a variety of cytotoxins, to find a combination that provides maximal effectiveness.

In another embodiment, an antibody identified by epitope binning and limiting antigen dilution as being a high-affinity antibody against an antigen of interest may be evaluated for its potential usefulness in a therapeutic product designed to enhance target cell function or otherwise confer a beneficial effect on a mammal, preferably a human. The antibody may be evaluated for its ability act directly on cells to bring out the desired effect and/or it may be evaluated for its suitability for use a conjuated form. For example, an antibody may be tested for its ability to bind to a receptor in such a way that prevents toxin binding to the receptor, or for its ability to bind to and neutralize a toxin. Alternately, an antibody may be tested for its ability to bind to and stimulate an effector molecule in a way that brings about a desired effect in a target cell or, if the effector is a circulating molecule, throughout an organism. An antibody may be evaluated for its ability to deliver a stimulant to a target cell, such that the stimulant may exert its desired effect on the target cell.

An advantageous aspect of the present invention provides methods for assessing the potential usefulness of antibodies for use in immunotoxins by screening, categorizing, and ranking conjugated antibodies. Antibodies may be conjugated with a cytotoxin or with some other label, after the antibodies are recovered and before the epitope binning and limiting antigen dilution assays are carried out. By using conjugated antibodies to practice the methods of the invention, this method provides an effective method for identifying and isolating antibodies in which high-affinity epitope binding is not hindered by the presence of a toxin or other label. In one embodiment, conjugation reactions are carried out using antibody-containing hybridoma supernatants, such that the antibodies are conjugated to a cytotoxin of interest. A panel of conjugated antibodies are then “binned” and kinetically ranked, to identify those conjugated antibodies that have high affinity for an epitope of interest. In other embodiments, the antibodies in hybridoma supernatants may be conjugated to a protein or carbohydrate label, or even to a cross-linking group alone.

Another advantageous aspect of the present invention provides a method for screening, binning, and ranking a heterogeneous panel of antibodies generated by challenge with a single antigen, with the result that the heterogeneous panel is sorted into groups of antibodies against different epitopes on the same antigen. This makes it possible to simultaneously study the characteristics of the highest-affinity antibodies against different epitopes on the same antigen. By comparing the effects of antibodies against different epitopes, it may be possible to identify which epitopes are better targets for therapeutic products directed against a particular antigen. In one embodiment, a panel of hundreds of antibodies is raised against the extracellular domain of a tumor-specific member of a growth factor receptor family. Using epitope binning and limiting antigen dilution assays, the highest-affinity antibodies against various epitopes on the receptor are identified, screened for their ability to inhibit ligand binding to the receptor, and compared to determine which antibody shows the greatest ability to inhibit receptor function.

Antibodies from different sources can be combined for use in the methods of the present invention. For example, antibodies obtained from different individuals or cell cultures that were subjected to challenge with the same antigen, or polyclonal and monoclonal antibodies raised against the same antigen can be combined to screen, categorize, rank, and evaluate antibodies using the methods of the present invention.

Preferably, the methods of the invention are used to screen human, chimeric or humanized antibodies to provide therapeutic products that avoid rejection when used in human subjects. Although mice are convenient for immunization and recognize most human antigens as foreign such that murine antibodies against human targets with therapeutic potential can be generated, these advantages are overshadowed by disadvantages such as a higher dosing requirement, a shorter circulating half-life, and the possibility of eliciting human antibodies against the murine antibodies. Preferably, human or humanized antibodies are produced using the transgenic XenoMouse™ maintained by available cloning vehicles. The use of yeast artificial chromosome (YAC) cloning vectors led the way to introducing large germline fragments of human Ig locus into transgenic mammals. Essentially a majority of the human V, D, and J region genes arranged with the same spacing found in the human genome and the human constant regions were introduced into mice using YACs. One such transgenic mice is known as XenoMouse and is commercially available from Abgenix, Inc. (Fremont Calif.).

A XenoMouse is a mouse which has inactivated mouse IgH and IgK loci and is transgenic for functional megabase-sized human IgH and IgK transgenes. Further, the XenoMouse is a transgenic mouse capable of producing high affinity, fully human antibodies of the desired IgGI isotype in response to immunization with virtually any desired antigen. Such a mAbs can be used to direct complement dependent cytotoxicity or antibody-dependent cytotoxicity to a target cell.

Cancer

One aspect of the present invention provides methods to identify potentially therapeutic antibodies directed against cancer antigens, preferably against antigens associated with solid tumors. In various preferred Embodiments, the methods of the present invention can be used to identify antibodies directed against antigens associated with prostate, kidney, bladder, lung, colon, and ovarian cancers, and in particular against prostate stem cell antigen (PSCA).

Another aspect of the present invention provides methods to identify therapeutic products for cancer therapy, by identifying, categorizing, and ranking antibodies having a high affinity for, and a low dissociation rate from, its antigen. In one embodiment, antibodies can be identified that act directly on cancer cells, for example by inducing apoptosis (programmed cell death) or inhibition of cell proliferation, by binding with high affinity to the relevant antigens. In another embodiment, antibodies may work synergistically with the host's immune effector mechanisms, for example to enhance antibody-dependent cellular cytotoxicity (ADCC) and complement-dependent cytolysis. In another embodiment, methods of the present invention may be used to identify antibodies with potential use in immunotoxins, whereby the specificity and high affinity of the antibody for a cancer-associated antigen permits delivery of the conjugated toxin to the cancer cell. Preferably, the antibodies are specific for antigens associated with solid tumors, prostate, kidney, bladder, lung, colon, or ovarian cancers, and in particular for prostate stem cell antigen (PSCA).

Definitions

Unless defined otherwise, technical and scientific terms used herein have the same meaning as commonly understood by one of ordinary skill in the art to which this invention belongs. See, e.g. Singleton et al., Dictionary of Microbiology and Molecular Biology 2^{nd} ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present invention, the following terms are defined below.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

“Native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Chothia et al. J Mol. Biol. 186:651 (1985; Novotny and Haber, Proc. Natl. Acad. Sci. U.S.A. 82:4592 (1985); Chothia et al., Nature 342:877-883 (1989)).

The term “antibody” herein is used in the broadest sense and specifically covers intact monoclonal antibodies, polyclonal antibodies, multi-specific antibodies (e.g. bi-specific antibodies) formed from at least two intact antibodies, chimeric antibodies, and antibody fragments, so long as they exhibit the desired biological activity. The term “antibody” includes all classes and subclasses of intact immunoglobulins.

Depending on the amino acid sequence of the constant domain of their heavy chains, intact antibodies can be assigned to different “classes”. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The “light chains” of antibodies (immunoglobulns) from any vertebrate species can be assigned to one of two clearly distinct types, called κ and λ, based on the amino acid sequences of their constant domains. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single epitope on a single antigen. Monoclonal antibodies are advantageous for use in the present invention in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al, Nature, 352:624-628 (1991) and Marks et al., J Mol. Biol., 222:581-597 (1991), for example.

The term “chimeric antibody” as used herein refers to antibodies containing, or encoded by, materials derived from more than one source. For example, a chimeric antibody may contain regions derived from mouse antibodies combined with regions derived from human antibodies to produce an antibody have certain desired characteristics. Alternately, a chimeric antibody may be an antibody encoded by a chimeric gene that may contain coding regions obtained from different species or coding regions obtained from different members of the same species or coding regions from different regions of the same genome, in order to generate a gene product having certain desired characteristics. A humanized antibody may be considered a chimeric antibody within this definition.

An “isolated” antibody is one which has been identified and separated and/or recovered from a component of its natural environment. As used herein, an isolated antibody may be an antibody secreted into the medium of a culture of antibody-producing cells, e.g., a B cell culture or a hybridoma culture, preferably where the cultured cells are have been centrifuged and the medium containing antibodies is collected as a supernatant.

By “neutralizing antibody” is meant an antibody molecule which is able to eliminate or significantly reduce an effector function of a target antigen to which is binds. Accordingly, a therapeutic product that acts as a “neutralizing” antibody is capable of eliminating or significantly reducing an effector function.

“Antibody-dependent cell-mediated cytotoxicity” and “ADCC” refer to a cell-mediated reaction in which non-specific cytotoxic cells that express Fc receptors (FcRs) (e.g. Natural Killer (NK) cells, neutrophils, and macrophages) recognize bound antibody on a target cell and subsequently cause lysis of the target cell. To assess ADCC activity of a molecule of interest, an in vitro ADCC assay, such as that described in U.S. Pat. No. 5,500,362, or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1988).

The term “epitope” is used to refer to binding sites for (monoclonal or polyclonal) antibodies on protein antigens.

The term “therapeutic product” refers to a product used to treat a disorder or disease state in a mammal, as well as to a product administered for its beneficial effects in the absence of any apparent disorder or disease state. As used herein, a “therapeutic product” contains an antibody or antibody fragment. A therapeutic product may be a therapeutic antibody containing an antibody or antibody fragment and if needed, carriers, buffers, excipients and the like. Alternately, a therapeutic product may contain an antibody or antibody fragment conjugated to at least one bioactive substance such as a cytotoxin or a stimulant, and if needed, carriers, buffers, excipients and the like. The term “immunotoxin” refers to a therapeutic product containing an antibody conjugated to at least one cytotoxin, where the antibody and cytoxin(s) may be conjugated or combined by any suitable means, with or without the use of cross-linking agents. An immunotoxin may be used to deliver a toxin to a target cell, in order to destroy or inhibit the target cell. A therapeutic product containing an antibody conjugated to or otherwise combined with a stimulant may be used to stimulate or enhance the functioning of a target cell.

The term “disease state” refers to a physiological state of a cell or of a whole mammal in which an interruption, cessation, or disorder of cellular or body functions, systems, or organs has occurred.

The term “treat” or “treatment” refer to both therapeutic treatment and prophylactic or preventative measures, wherein the object is to prevent or slow down (lessen) an undesired physiological change or disorder, such as the development or spread of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

A “disorder” is any condition that would benefit from treatment of the present invention. This includes chronic and acute disorders or disease including those pathological conditions which predispose the mammal to the disorder in question. Non-limiting examples of disorders to be treated herein include benign and malignant tumors, leukemias and lymphoid malignancies, in particular breast, rectal, ovarian, stomach, endometrial, salivary gland, kidney, colon, thyroid, pancreatic, prostate or bladder cancer. A preferred disorder to be treated in accordance with the present invention is malignant tumor, such as cervical carcinomas and cervical intraepithelial squamous and glandular neoplasia, renal cell carcinoma (RCC), esophageal tumors, and carcinoma-derived cell lines.

“Tumor”, as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

“Mammal” for purposes of treatment refers to any animal classified as a mammal, including humans, domestic and farm animals, and zoo, sports, or pet animals, such as dogs, horses, cats, cows, etc. Preferably, the mammal is human.

### Epitope Binning

With increased fusion efficiency producing larger numbers of antigen specific antibodies from each hybridoma-cell fusion experiment, a screening method of managing and prioritizing large numbers of antibodies becomes ever more important. When a set of monoclonal antibodies has been generated against a target antigen, different antibodies in the set will recognize different epitopes, and will also have variable binding affinities. Thus, to effectively screen large numbers of antibodies it is important to determine which epitope each antibody binds, and to determine binding affinity for each antibody.

Epitope binning, as described herein, is the process of grouping antibodies based on the epitopes they recognize. More particularly, epitope binning comprises methods and systems for discriminating the epitope recognition properties of different antibodies, combined with computational processes for clustering antibodies based on their epitope recognition properties and identifying antibodies having distinct binding specificities. Accordingly, embodiments include assays for determining the epitope binding properties of antibodies, and processes for analyzing data generated from such assays.

In general, the invention provides an assay to determine whether a test moiety (such as an antibody) binds to a test object (such as an antigen) in competition with other test moieties (such as other antibodies). A capture moiety is used to capture the test object and/or the test moiety in an addressable manner and a detection moiety is utilized to addressably detect binding between other test moieties and the test object. When a test moiety binds to the same or similar location on the test subject as the test moiety being assayed, no binding is detected, whereas when a test moiety binds to a different location on the test subject as the test moiety being assayed, binding is detected. In each case, the binding or lack thereof is addressable, so the relative interactions between test moieties with the test object can be readily ascertained and categorized.

One embodiment of the invention is a competition-based method of categorizing a set of antibodies that have been generated against an antigen. This method relies upon carrying out a series of assays wherein each antibody from the set is tested for competitive binding against all other antibodies from the set. Thus, each antibody will be used in two different modes: in at least one assay, each antibody will be used in “detect” mode as the “probe antibody” that is tested against all the other antibodies in the set; in other assays, the antibody will be used in “capture” mode as a “reference antibody” within the set of reference antibodies being assayed. Within the set of reference antibodies, each reference antibody will be uniquely labelled in a way that permits detection and identification each reference antibody within a mixture of reference antibodies. The method relies on forming “sandwiches” or complexes involving reference antibodies, antigen, and probe antibody, and detecting the formation or lack of formation of these complexes. Because each reference antibody in the set is uniquely labelled, it is possible to addressably determine whether a complex has formed for each reference antibody present in the set of reference antibodies being assayed.

### Antibody Assay Overview

The method begins by selecting an antibody from the set of antibodies against an antigen, where the selected antibody will serve as the “probe antibody” that is to be tested for competitive binding against all other antibodies of the set. A mixture containing all the antibodies will serve as a set of “reference antibodies” for the assay, where each reference antibody in the mixture is uniquely labelled. In an assay, the probe antibody is contacted with the set of reference antibodies, in the presence of the target antigen. Accordingly, a complex will form between the probe antibody and any other antibody in the set that does not compete for the same epitope on the target antigen. A complex will not form between the probe antibody and any other antibody in the set that competes for the same epitope on the target antigen Formation of complexes is detected using a labelled detection antibody that binds the probe antibody. Because each reference antibody in the mixture is uniquely labelled, it is possible to determine for each reference antibody whether that reference antibody does or does not form a complex with the probe antibody. Thus, it can be determined which antibodies in the mixture compete with the probe antibody and bind to the same epitope as the probe antibody.

Each antibody is used as the probe antibody in at least one assay. By repeating this method of testing each individual antibody in the set against the entire set of antibodies, the competitive binding affinities can be generated for the entire set of antibodies against an antigen. From such a affinity measurements, one can determine which antibodies in the set have similar binding characteristics to other antibodies in the set, thereby allowing the grouping or “binning” of each antibody on the basis of its epitope binding profile. A table of competitive binding affinity measurements is a suitable method for displaying assay results. A preferred embodiment of this method is the Multiplexed Competitive Antibody Binning (MCAB) assay for high-throughput screening of antibodies.

Because this embodiment relies on testing antibody competition, wherein a single antibody is tested against the entire set of antibodies generated against an antigen, one challenge to implementing this method relates to the mechanism used to uniquely identify and quantitatively measure complexes formed between the single antibody and any one of the other antibodies in the set. It is this quantitative measurement that provides an estimate of whether two antibodies are competing for the same epitope on the antigen.

As described below, embodiments of the invention relate to uniquely labelling each reference antibody in the set prior to creating a mixture of all antibodies. This unique label, as discussed below, is not limited to any particular mechanism. Rather, it is contemplated that any method that provides a way to identify each reference antibody within the mixture, allowing one to distinguish each reference antibody in the set from every other reference antibody in the set, would be suitable. For example, each reference antibody can be labelled calorimetrically so that the particular color of each antibody in the set is determinable. Alternatively, each reference antibody in the set might be labelled radioactively using differing radioactive isotopes. The reference antibody may be labelled by coupling, linking, or attaching the antibody to a labelled object such as a bead or other surface.

Once each reference antibody in the set has been uniquely labelled, a mixture is formed containing all the reference antibodies. Antigen is added to the mixture, and the probe antibody is added to the mixture. A detection label is necessary in order to detect complexes containing bound probe antibody. A detection label may be a labelled detection antibody or it may be another label that binds to the probe antibody. For example, when a set of human monoclonal antibodies is being tested, a mouse anti-human monoclonal antibody is suitable for use as a detection antibody. The detection label is chosen to be distinct from all other labels in the mixture that are used to label reference antibodies. For example, a labelled detection antibody might be labelled with a unique color, or radioactively labelled, or labelled by a particular fluorescent marker such as phycoerythrin (PE).

The design of an experiment must include selecting conditions such that the detection antibody will only bind to the probe antibody, and will not bind to the reference antibodies. In embodiments in which reference antibodies are coupled to beads or other materials through antibodies, the antibody that couples the reference antibody to the bead (the “capture antibody”) will be the same antibody as the detection antibody. In accordance with this embodiment of the invention, the detection antibody is specifically chosen or modified so that the detection antibody binds only to the probe antibody and does not bind to the reference antibody. By using the same antibody for both detection and capture, each will block one the other from binding to their respective targets. Accordingly, when the capture antibody is bound to the reference antibody, it will block the detection antibody from binding to the same epitope on the reference antibody and producing a false positive result. Antibodies suitable for use as detection antibodies include mouse anti-human IgG2, IgG3, and IgG4 antibodies available from Calbiochem, (Catalog No. 411427, mouse anti-human IgKappa available from Southern Biotechnology Associates, Inc. (Catalog Nos. 9220-01 and 9220-08, and mouse anti-hlgG from PharMingen (Catalog Nos. 555784 and 555785).

Once the labelled detection antibody has been added to the mixture, the entire mixture can then be analyzed to detect complexes between labelled detection antibody, bound probe antibody, the antigen, and uniquely labelled reference antibody. The detection method must permit detection of complexes (or lack thereof for each uniquely labelled reference antibody in the mixture.

Detecting whether a complex formed between a probe antibody and each reference antibody in the set indicates, for each reference antibody, whether that reference antibody competes with the probe antibody for binding to the same (or nearby) epitope. Because the mixture of reference antibodies will include the antibody being used as the probe antibody, it is expected that this provides a negative control. Detecting complex formation allows measurement of competitive affinities of the antibodies in the set being tested. This measurement of competitive affinities is then used to categorize each antibody in the set based on how strongly or weakly they bind to the same epitopes on the target antigen. This provides a rapid method for grouping antibodies in a set based on their binding characteristics.

In one embodiment, large numbers of antibodies can be simultaneously screened for their epitope recognition properties in a single experiment in accordance with embodiments of the present invention, as described below. Generally, the term “experiment” is used nonexclusively herein to indicate a collection of individual antibody assays and suitable controls. The term “assay” is used nonexclusively herein to refer to individual assays, for example reactions carried out in a single well of a microtiter plate using a single probe antibody, or may be used to refer to a collection of assays or to refer to a method of measuring antibody binding and competition as described herein.

In one embodiment, large numbers of antibodies are simultaneously screened for their epitope recognition properties using a sandwich assay involving a set of reference antibodies in which each reference antibody in the set is bound to a uniquely labelled “capture” antibody. The capture antibody can be, for example, a calorimetrically labelled antibody that has strong affinity for the antibodies in the set. As one example, the capture antibody can be a labelled mouse, goat, or bovine anti-human IgG or anti-human IgKappa antibody. Although embodiments described herein use a mouse monoclonal anti-human IgG antibody, other similar capture antibodies that will bind to the antibodies being studied are within the scope of the invention. Thus, one of skill in the art can select an appropriate capture antibody based on the origin of the set of antibodies being tested.

One embodiment of the present invention therefore provides a method of categorizing, for example, which epitopes on a target antigen are bound by fifty (50) different antibodies generated against that target antigen. Once the 50 antibodies have been determined to have some affinity for a target antigen, the methods described below are used to determine which antibodies in the group of 50 bind to the same epitope. These methods are performed by using each one of the 50 antibodies as a probe antibody to cross-compete against a mixture of all 50 antibodies (the reference antibodies), wherein the 50 uniquely labelled reference antibodies in the mixture are each labelled by a capture antibody. Those antibodies that recognize the same epitope will compete with one another, while antibodies that do not compete are assumed to not bind to the same epitope. By uniquely labelling a large number of antibodies in a single reaction, as described below, these methods allow for a pre-selected antibody to be competed against 10, 25, 50, 100, 200, 300, or more antibodies at one time. For this reason, the choice of testing 50 antibodies in an experiment is arbitrary, and should not be viewed as limiting on the invention.

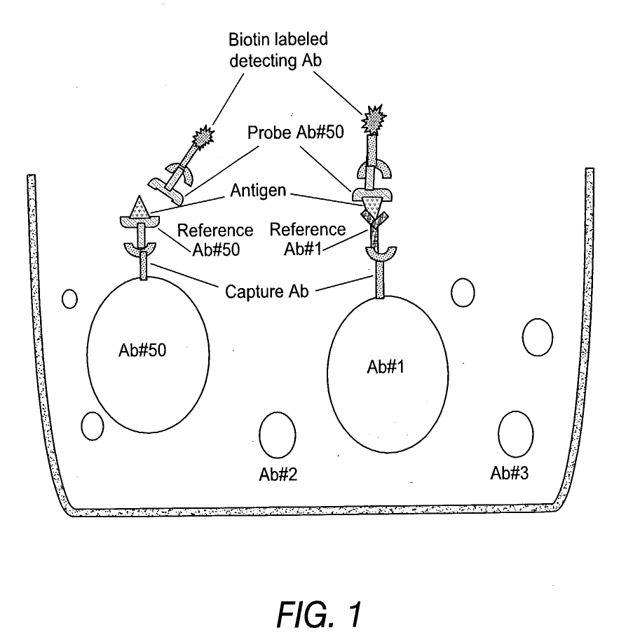

Preferably, the Multiplex Competitive Antibody Binning (MCAB) assay is used. More preferably, the MCAB assay is practiced utilizing the LUMINEX System (Luminex Corp., Austin Tex.), wherein up to 100 antibodies can be binned simultaneously using the method illustrated in FIG. 1. The MCAB assay is based on the competitive binding of two antibodies to a single antigen molecule. The entire set of antibodies to be characterized is used twice in the MCAB assay, in “capture” and “detect” modes in the MCAB sandwich assay.

In one embodiment, each capture antibody is uniquely labelled. Once a capture antibody has been uniquely labelled, it is exposed to one of the set of antibodies being tested, forming a reference antibody that is uniquely labelled. This is repeated for the remaining antibodies in the set so that each antibody becomes labelled with a different colored capture antibody. For example, when 50 antibodies are being tested, a labelled reference antibody mixture is created by mixing all 50 uniquely labelled reference antibodies into a single reaction well. For this reason, it is useful for each label to have a distinct property that allows it to be distinguished or detected when mixed with other labels. In one preferred embodiment, each capture antibody is labelled with a distinct pattern of fluorochromes so they can be calorimetrically distinguished from one another.

Once the test antibody mixture is created, it is placed into multiple wells of, for example, a microtiter plate. In this example, the same antibody mixture would be placed in each of 50 microtiter wells and the mixture in each well would then be incubated with the target antigen as a first step in the competition assay. After incubation with the target antigen, a single probe antibody selected from the original set of 50 antibodies is added to each well. In this example, only one probe antibody is added to each reference antibody mixture. If any labelled reference antibody in the well binds to the target antigen at the same epitope as the probe antibody, they will compete with one another for the epitope binding site.

It is understood by one of skill in the art that embodiments of the invention are not limited to only adding a single probe antibody to each well. Other methods wherein multiple probe antibodies, each one distinguishably labelled from one another, are added to the mixture are contemplated.

In order to determine whether the probe antibody has bound to any of the 50 labelled reference antibodies in the well, a labelled detection antibody is added to each of the 50 reactions. In one embodiment, the labelled detection antibody is a differentially labelled version of the same antibody used as the capture antibody. Thus, for example, the detection antibody can be a mouse anti-human IgG antibody or a anti-human IgKappa antibody. The detection antibody will bind to, and label, the probe antibody that was placed in the well.

The label on the detection antibody permits detection and measurement of the amount of probe antibody bound to a complex formed by a reference antibody, the antigen, and the probe antibody. This complex serves as a measurement of the competition between the probe antibody and the reference antibody. The detection antibody may be labelled with any suitable label which facilitates detection of the secondary antibody. For example, a detection antibody may be labelled with biotin, which facilitates fluorescent detection of the probe antibody when streptavidin-phycoerythrin (PE) is added. The detection antibody may be labelled with any label that uniquely determines its presence as part of a complex, such as biotin, digoxygenin, lectin, radioisotopes, enzymes, or other labels. If desired, the label may also facilitate isolation of beads or other surfaces with antibody-antigen complexes attached.

The amount of labelled detection antibody bound to each uniquely labelled reference antibody indicates the amount of bound probe antibody, and the labelled detection antibody is bound to the probe antibody bound to antigen bound to labelled reference antibody. Measuring the amount of labelled detection antibody bound to each one of the 50 labelled reference antibodies indicates the amount of bound probe antibody can be obtained, where the amount of bound probe antibody is an indicator of the similarity or dissimilarity of the epitope recognition properties of the two antibodies (probe and reference). If a measurable amount of the labelled detection antibody is detected on the labelled reference antibody-antigen complex, that is understood to indicate that the probe antibody and the reference antibody do not bind to the same epitope on the antigen. Conversely, if little or no measurable detection antibody is detected on the labelled reference antibody-antigen complex, then it is understood to indicate that the probe antibody for that reaction bound to very similar or identical epitopes on the antigen. If a small amount of detection antibody is detected on the reference antibody-antigen complex, that is understood to indicate that the reference and probe antibodies may have similar but not identical epitope recognition properties, e.g., the binding of the reference antibody to its epitope interferes with but does not completely inhibit binding of the probe antibody to its epitope.

Another aspect of the present invention provides a method for detecting both the reference antibody and the amount of probe antibody bound to an antigen. If antibody complexes containing different reference antibodies have been mixed, then the unique property provided by the unique labels on the capture antibody can be used to identify the reference antibody coupled to that bead. Preferably, that distinct property is a unique emission spectrum.

The amount of probe antibody bound to any reference antibody can be determined by measuring the amount of detection label bound to the complex. The detection label may be a labelled detection antibody bound to probe antibody bound to the complex, or it may be a label attached to the probe antibody. Thus, the epitope recognition properties of both a reference antibody and a probe antibody can be measured by using a comparative measure of the competition between the two antibodies for an epitope.

Conditions for optimizing procedures can be determined by empirical methods and knowledge of one of skill in the art. Incubation time, temperature, buffers, reagents, and other factors can be varied until a sufficiently strong or clear signal is obtained. For example, the optimal concentration of various antibodies can be empirically determined by one of skill in the art, by testing antibodies and antigens at different concentrations and looking for the concentration that produces the strongest signal or other desired result. In one embodiment, the optimal concentration of primary and secondary antibodies—that is, antibodies to be binned—is determined by a double titration of two antibodies raised against different epitopes of the same antigen, in the presence of a negative control antibody that does not recognize the antigen.

### Assays Using Colored Beads

In a preferred embodiment, large numbers of antibodies are simultaneously screened for their epitope recognition properties in a single assay using color-coded microspheres or beads to identify multiple reactions in a single tube or well, preferably using a system available from Luminex Corporation (Luminex Corp, Austin Tex.), and most preferably using the Luminex 100 system. Preferably, the MCAB assay is carried out using Luminex technology. In another preferred embodiment, up to 100 different antibodies to be tested are bound to Luminex beads with 100 distinct colors. This system provides 100 different sets of polystyrene beads with varying amounts of fluorochromes embedded. This gives each set of beads a distinct fluorescent emission spectrum and hence a distinct color code.

To characterize the binding properties of antibodies using the Luminex 100 system, beads are coated with a capture antibody which is covalently attached to each bead; preferably a mouse anti-human IgG or anti-human IgKappa monoclonal antibody is used. Each set of beads is then incubated in a well containing a reference antibody to be characterized (e.g., containing hybridoma supernatant) such that a complex if formed between the bead, the capture antibody, and the reference antibody (henceforth, a “reference antibody-bead” complex) which has a distinct fluorescence emission spectrum and hence, a color code, that provides a unique label for that reference antibody.

In this preferred embodiment, each reference antibody-bead complex from each reaction with each reference antibody is mixed with other reference antibody-bead complexes to form a mixture containing all the reference antibodies being tested, where each reference antibody is uniquely labelled by being couple to a bead. The mixture is aliqotted into as many wells of a 96-well plate as is necessary for the experiment. Generally, the number of well will be determined by the number of probe antibodies being tested, along with various controls. Each of these wells containing an aliquot of the mixture of reference antibody-bead complexes is incubated first with antigen and then probe antibody (one of the antibodies to be characterized), and then detection antibody (a labelled version of the original capture antibody), where the detection antibody is used for detection of bound probe antibody. In a preferred embodiment, the detection antibody is a biotinylated mouse anti-human IgG monoclonal antibody. This process is illustrated in FIG. 1.

In the illustrative embodiment presented in FIG. 1, each reference antibody is coupled to a bead with distinct emission spectrum, where the reference antibody is coupled through a mouse anti-human monoclonal capture antibody, forming a uniquely labelled reference antibody. The entire set of uniquely labelled reference antibodies is placed in the well of a multiwell microtiter plate. The set of reference antibodies are incubated with antigen, and then a probe antibody is added to the well. A probe antibody will only bind to antigen that is bound to a reference antibody that recognizes a different epitope. Binding of a probe antibody to antigen will form a complex consisting of a reference antibody coupled to a bead through a capture antibody, the antigen, and the bound probe antibody. A labelled detection antibody is added to detect bound probe antibody. Here, the detection antibody is labelled with biotin, and bound probe antibody is detected by the interaction of streptavidin-PE and the biotinylated detection antibody. As shown in FIG. 1, Antibody #50 is used as the probe antibody, and the reference antibodies are Antibody #50 and Antibody #1. Probe Antibody #50 will bind to antigen that is bound to reference Antibody #1 because the antibodies bind to different epitopes, and a labelled complex can be detected. Probe antibody #50 will not bind to antigen that is bound by reference antibody #50 because both antibodies are competing for the same epitope, such that no labelled complex is formed.

In this embodiment, after the incubation steps are completed, the beads of a given well are aligned in a single file in a cuvette and one bead at a time passes through two lasers. The first laser excites fluorochromes embedded in the beads, identifying which reference antibody is bound to each bead. A second laser excites fluorescent molecules bound to the bead complex, which quantifies the amount of bound detection antibody and hence, the amount of probe antibody bound to the antigen on a reference antibody-bead complex. When a strong signal for the detection antibody is measured on a bead, that indicates the reference and probe antibodies bound to that bead are bound to different sites on the antigen and hence, recognize different epitopes on the antigen. When a weak signal for the bound detection antibody is measured on a bead, that indicates the corresponding reference and probe antibodies compete for the same epitope. This is illustrated in FIG. 1. A key advantage of this embodiment is that it can be carried out in high-throughput mode, such that multiple competition assays can be simultaneously performed in a single well, saving both time and resources.

The assay described herein may include measurements of at least one additional parameter of the epitope recognition properties of primary and secondary antibodies being characterized, for example the effect of temperature, ion concentration, solvents (including detergent) or any other factor of interest. One of skill in the relevant art can use the present disclosure to develop an experimental design that permits the testing of at least one additional factor. If necessary, multiple replicates of an assay may be carried out, in which factors such as temperature, ion concentration, solvent, or others, are varied according to the experimental design. When additional factors are tested, methods of data analysis can be adjusted accordingly to include the additional factors in the analysis.

### Data analysis

Another aspect of the present invention provides processes for analyzing data generated from at least one assay, preferably from at least one high throughput assay, in order to identify antibodies having similar and dissimilar epitope recognition properties. A comparative approach, based on comparing the epitope recognition properties of a collection of antibodies, permits identification of those antibodies having similar epitope recognition properties, which are likely to compete for the same epitope, as well as the identification of those antibodies having dissimilar epitope recognition properties, which are likely to bind to different epitopes. In this way, antibodies can be categorized, or “binned” based on which epitope they recognize. A preferred embodiment provides the Competitive Pattern Recognition (CPR) process for analyzing data generated by a high throughput assay. More preferably, CPR is used to analyze data generated by the Multiplexed Competitive Antibody Binning (MCAB) high-throughput competitive assay. Application of data analysis processes as disclosed and claimed herein makes it possible to eliminate redundancy by identifying the distinct binding specificities represented within a pool of antigen-specific antibodies characterized by an assay such as the MCAB assay.

A preferred embodiment of the present invention provides a process that clusters antibodies into “bins” or categories representing distinct binding specificities for the antigen target. In yet another preferred embodiment, the CPR process is applied to data representing the outcomes of the MCAB high-throughput competition assay in which every antibody competes with every other antibody for binding sites on antigen molecules. Embodiments carried out using different data sets of antibodies generated from XenoMouse animals provide a demonstration that application of the process of the present invention produces consistent and reproducible results.

The analysis of data generated from an experiment typically involves multi-step operations to normalize data across different wells in which the assay has been carried out and cluster data by identifying and classifying the competition patterns of the antibodies tested. A matrix-based computational process for clustering antibodies is then performed based on the similarity of their competition patterns, wherein the process is applied to classify sets of antibodies, preferably antibodies generated from hybridoma cells.

Antibodies that are clustered based on the similarity of their competition patterns are considered to bind the same epitope or similar epitopes. These clusters may optionally be displayed in matrix format, or in “tree” format as a dendrogram, or in a computer-readable format, or in any data-input-device-compatible format. Information regarding clusters may be captured from a matrix, a dendrogram or by a computer or other computational device. Data capture may be visual, manual, automated, or any combination thereof.

As used herein, the term “bin” may be used as a noun to refer to clusters of antibodies identified as having similar competition according to the methods of the present invention. The term “bin” may also be used a verb to refer to practicing the methods of the present invention. The term “epitope binning assay” as used herein, refers to the competition-based assay described herein, and includes any analysis of data produced by the assay.

Steps in data analysis are described in detail in the following disclosure, and practical guidance is provided by reference to the data and results are presented in Example 2. References to the data of Example 2, especially the matrices or dendrograms generated by performing various data analysis steps on the input data of Example 2, serve merely as illustrations and do not limit the scope of the present invention in any way.

When a large number and sizes of the data sets is generated, a systematic method is needed to analyze the matrices of signal intensities to determine which antibodies have similar signal intensity patterns. By way of example, two matrices containing m rows and m columns are generated in a single experiment, where m is the number of antibodies being examined. One matrix has signal intensities for the set of competition assays in which antigen is present. The second matrix has the corresponding signal intensities for a negative control experiment in which antigen is absent. Each row in a matrix represents a unique well in a multiwell microtiter plate, which identifies a unique probe antibody. Each column represents a unique bead spectral code, which identifies a unique reference antibody. The intensity of signal detected in each cell in a matrix represents the outcome of an individual competition assay involving a reference antibody and a probe antibody. The last row in the matrix corresponds to the well in which blocking buffer is added instead of a probe antibody. Similarly, the last column in the matrix corresponds to the bead spectral code to which blocking buffer is added instead of reference antibody. Blocking buffer serves as a negative control and determines the amount of signal present when only one antibody (of the reference-antibody-probe-antibody pair) is present.

Similar signal intensity value patterns for two rows indicate that the two probe antibodies exhibit similar binding behaviors, and hence likely compete for the same epitope. Likewise, similar signal intensity patterns for two columns indicate that the two reference antibodies exhibit similar binding behaviors, and hence likely compete for the same epitope. Antibodies with dissimilar signal patterns likely bind to different epitopes. Antibodies can be grouped, or “binned,” according to the epitope that they recognize, by grouping together rows with similar signal patterns or by grouping together columns with similar signal patterns. Such an assay described above is referred to as an epitope binning assay.

### Program to Apply Competitive Pattern Recognition (CPR) Process

One aspect of the present invention provides a program to apply the CPR process having two main steps: (1) normalization of signal intensities; and (2) generation of dissimilarity matrices and clustering of antibodies based on their normalized signal intensities. It is understood that the term “main step” encompasses multiple steps that may be carried as necessary, depending on the nature of the experimental material used and the nature of the data analysis desired. It is also understood that additional steps may be practiced as part of the present invention.

### Background Normalization of Signal Intensities

Input data is subjected to a series of preprocessing steps that improve the ability to detect meaningful patterns. Preferably, the input data comprises signal intensities stored in a two dimensional matrix, and a series of normalization steps are carried out to eliminate sources of noise or signal bias prior to clustering analysis.

The input data to be analyzed comprises the results from a complete assay of epitope recognition properties. Preferably, results comprise signal intensities measured from an assay carried out using labelled secondary antibodies. More preferably, results using the MCAB assay are analyzed as described herein. Two input files are generated: one input file from an assay in which antigen was added; and a second input file from an assay in which antigen was absent. The experiment in which antigen is absent serves as a negative control allowing one to quantify the amount of binding by the labelled antibodies that is not to the antigen. Preferably, each combination of primary antibody and secondary antibody being tested was assayed in the presence and absence of antigen, such that each combination is represented in both sets of input data. Even more preferably, the assay is carried out using the procedures for assaying epitope recognition properties of multiple antibodies using a multi-well format disclosed elsewhere in the present disclosure.

The input data normally comprises signal intensities stored in a two dimensional matrix. First, the matrix corresponding to the experiment without antigen (negative control) experiment, A_{B}, is subtracted from the matrix corresponding to the experiment with antigen, A_{E} to give the background normalized matrix given by A_{N}=A_{E}−A_{B}. This subtraction step eliminates background signal that is not due to binding of antibodies to antigen. The above matrices are of dimension (m+1)×(m+1) where m is the number of antibodies to be clustered. The last row and the last column contain intensity values for experiments in which blocking buffer was added in place of a probe antibody or reference antibody, respectively.

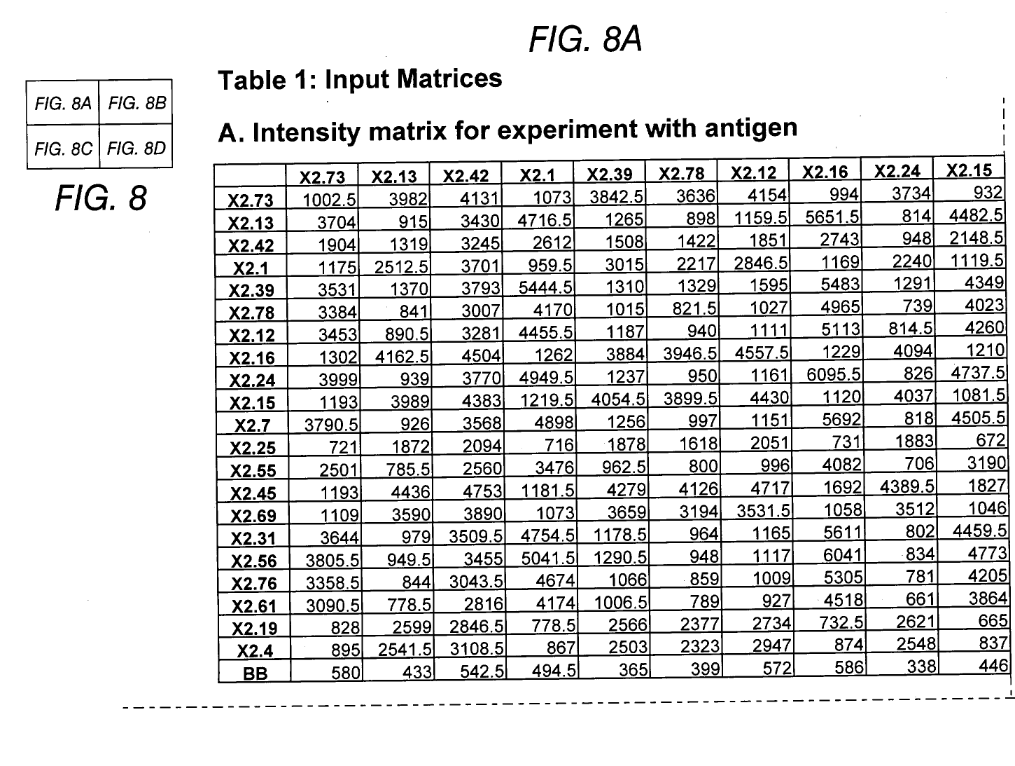

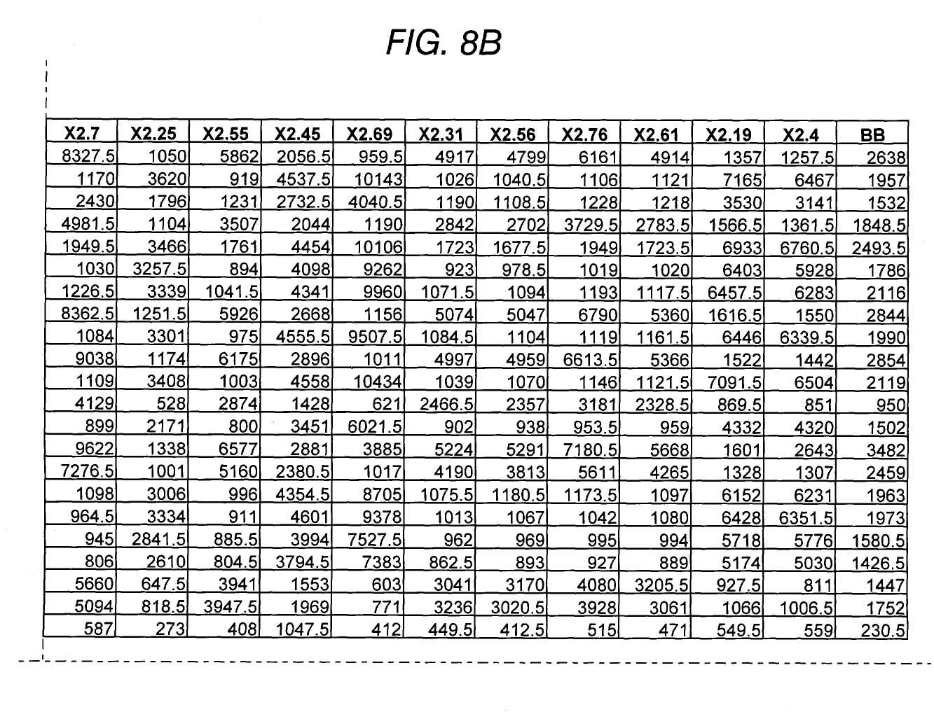

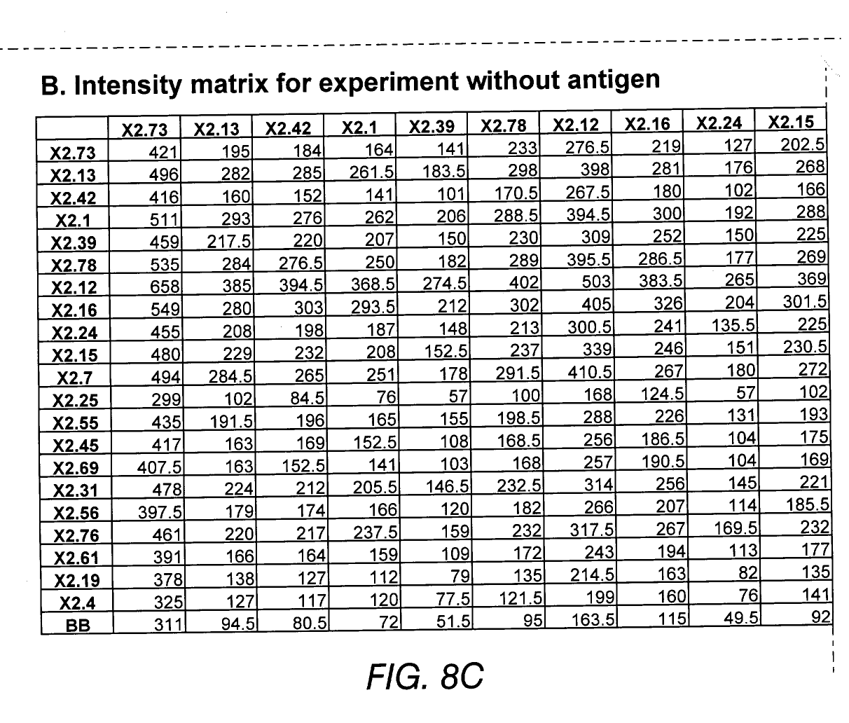

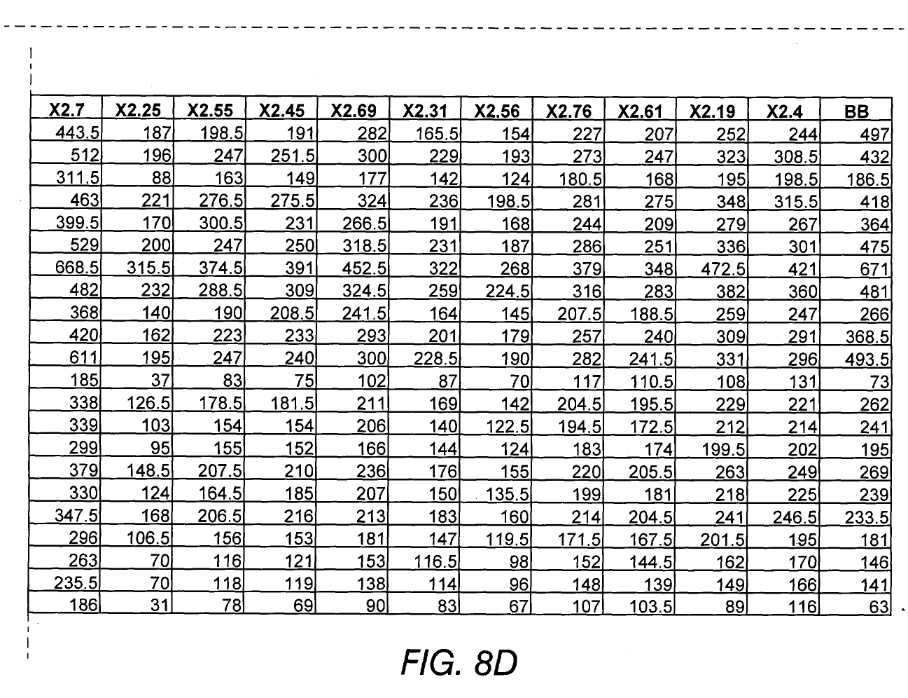

In an illustrative embodiment, FIG. 8A and 8B illustrate the intensity matrices generated in the embodiment disclosed in Example 2, which are used as input data matrices for subsequent steps in data analysis. FIG. 8A is the intensity matrix for an experiment conducted with antigen, and FIG. 8B is the intensity matrix for the same experiment conducted without antigen. Each row in the matrix corresponds to the signal intensities for the different beads in one well, where each well represents a unique detecting antibody. Each column represents the signal intensities corresponding to the competition of a unique primary antibody with each of the secondary antibodies. Each cell in the matrix represents an individual competition assay for a different pair of primary and secondary antibodies. In assays of epitope recognition properties, addition of blocking buffer in place of one of the antibodies serves as a negative control. In the embodiment illustrated by FIGS. 8A and 8B, the last row in the matrix corresponds to the well in which blocking buffer is added in place of a secondary antibody, and the last column in the matrix corresponds to the beads to which blocking buffer is added in place of primary antibody. Other arrangements of cells within a matrix can be used to practice aspects of the present invention, as one of skill in the relevant art can design data matrices having other formats and adapt subsequent manipulations of these data matrices to reflect the particular format chosen.

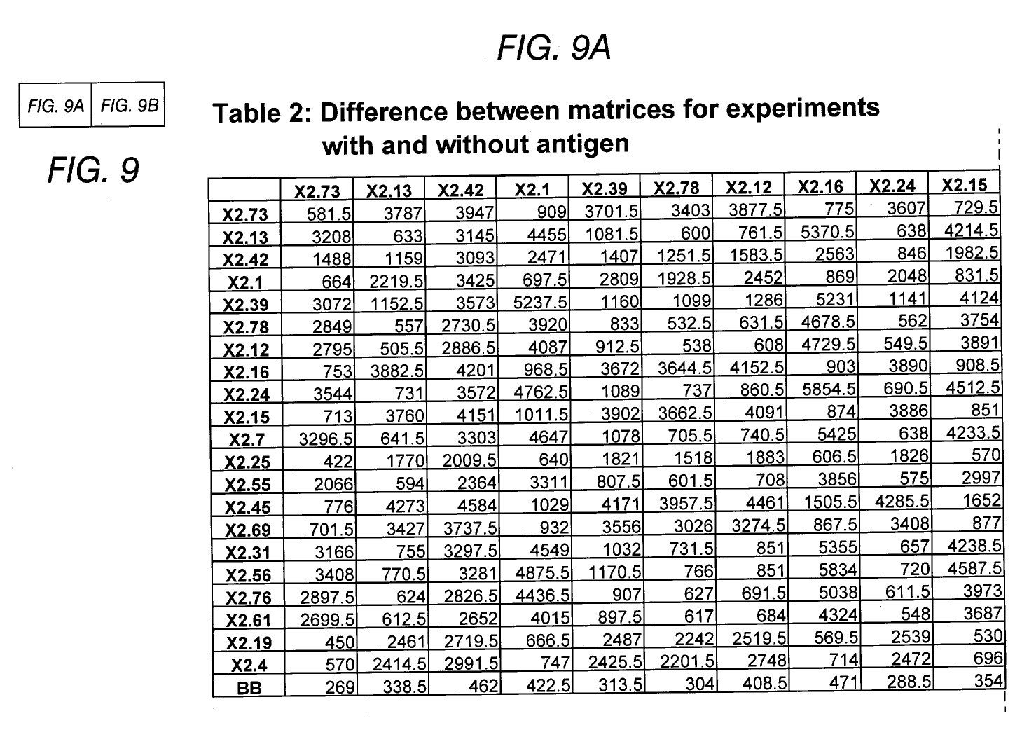

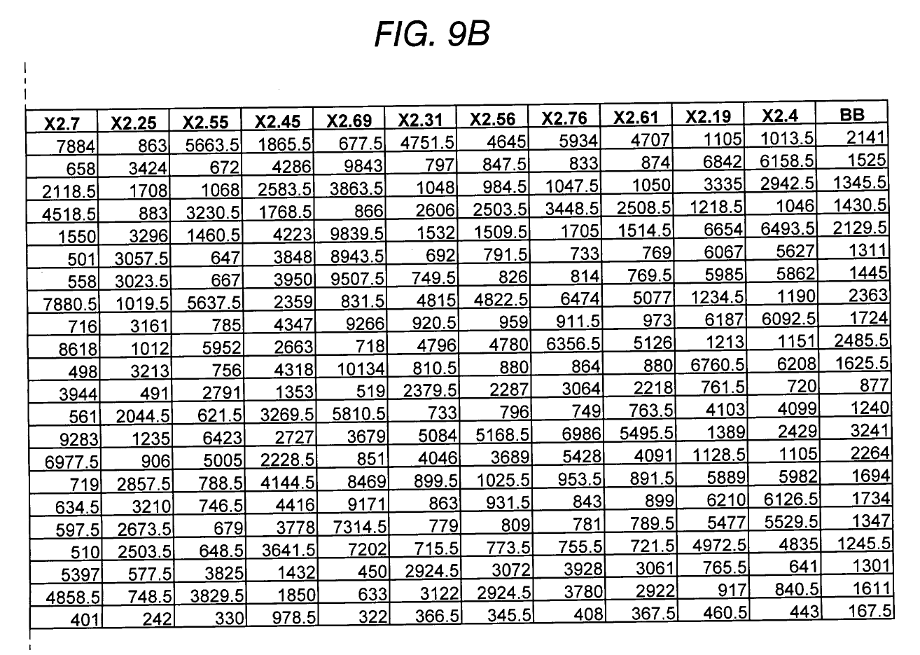

A different matrix can be generated by subtracting the matrix corresponding to values obtained from the experiment without antigen from the matrix corresponding to values obtained from the experiment with antigen. This step is performed to subtract from the total signal the amount of signal that is not attributed to the binding of the labelled probe antibody to the antigen. This subtraction step generates a difference matrix as illustrated in FIG. 9. Following this subtraction, any antibodies that have unusually high intensities for their diagonal values relative to the other diagonal values are flagged. High values for a column both along and off the diagonal suggest that the data associated with this particular bead may not be reliable. The antibodies corresponding to these columns are flagged at this step and are considered as individual bins.

### Elimination of Background Signals Due to Nonspecific Binding: Normalization of Signal Intensities Within Rows or Columns of the Matrix

In some cases, there is a significant disparity in the overall signal intensities between different rows or columns in the background-normalized signal intensity matrix. Row variations are likely due to variations in intensity from well to well, while column variation is likely due to the variation in the affinities and concentrations of different probe antibodies. In accordance with one aspect of the present invention, there is often a linear correlation between the blocking buffer values of the rows or columns, and the average signal intensity values of the rows or columns. If an intensity variation is observed, an additional step of row and/or column normalization is performed as described below.

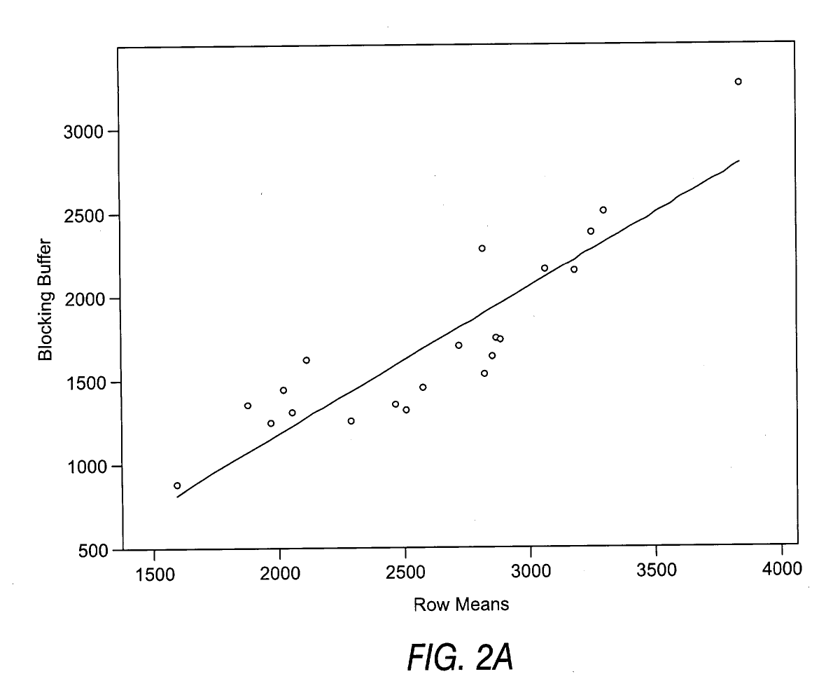

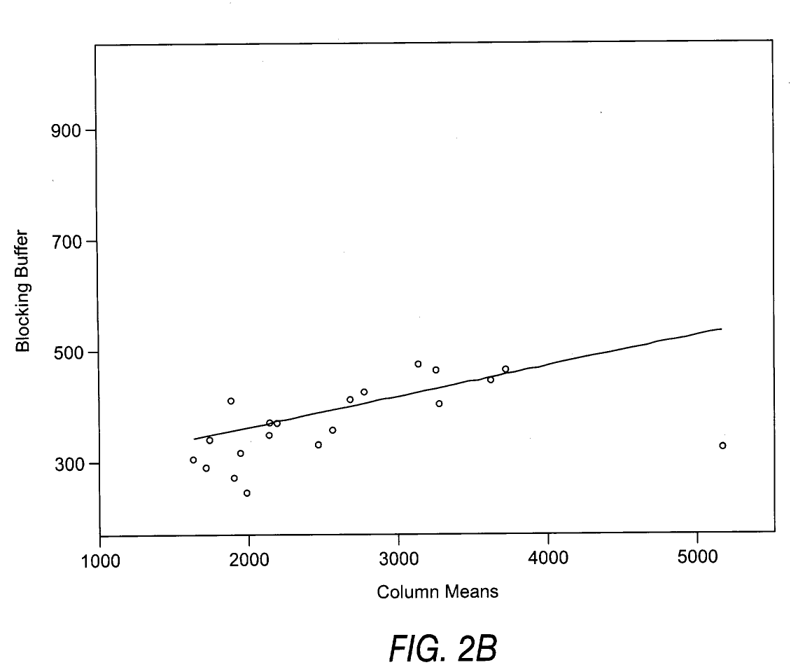

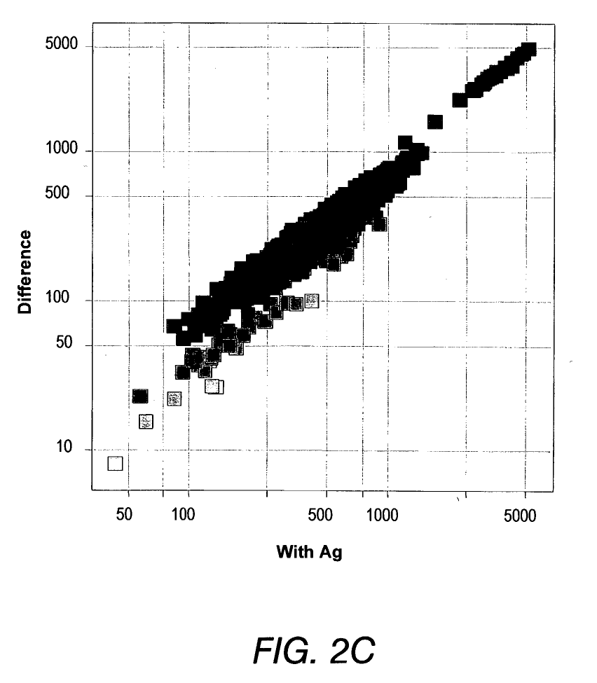

Row normalization. Row normalization is performed when there are any significant well-specific signal biases, and is carried out to eliminate any “signal artifacts” that would otherwise be introduced into the data analysis. One of skill in the art can determine whether the step is desirable based on the distribution of intensity values of the blocking buffer negative controls. By way of illustration, in FIG. 2A, the blocking buffer intensity value for each row is plotted against the average intensity value (excluding the blocking buffer value) for the corresponding row. The plot in FIG. 2A shows a clear linear correlation between the blocking buffer values and the average intensity value for a row. This figure shows that there is a well-specific signal bias in the samples being analyzed, and that the intensity value for the blocking buffer correlates to the overall signal intensity within a row. The different intensity biases seen in the different rows is likely due in part to the variation in affinity for the secondary antibodies for the antigen as well as the concentration variations of these secondary antibodies. Note that FIG. 2B shows that, for the same embodiment, there is weaker correlation between the blocking buffer intensity values for the columns and the average column intensity values.

For intensity variations in rows, the intensities of each row in the matrix are adjusted by dividing each value in a row by the blocking buffer intensity value for that row. In the case where blocking buffer data is absent, each row value is divided by the average intensity value for the row. In an embodiment applying the CPR process, the intensity-normalized matrix is given by

$${A}_{I} ( i , j ) = \frac{{A}_{N} ( i , j )}{I ( k )} 1 \leq i , j \leq m + 1$$

where I is a vector containing the blocking buffer or average intensities and k=i if normalization is done with respect to rows.

Column normalization. In this final pre-processing step, each column in the row normalized matrix (that was not flagged at the step the difference matrix was generated) is divided by its corresponding diagonal value. The cells along the diagonal represent competition assays for which the primary and secondary antibodies are the same. Ideally, values along the diagonal should be small as two copies of the same antibody should compete for the same epitope. The division of each column by its corresponding diagonal is done to measure each intensity relative to an intensity that is known to reflect competition—i.e., competition of an antibody against itself.

For intensity variations in columns, the intensities of each column in the matrix are adjusted by dividing each value in a column by the blocking buffer intensity value for that row. In the case where blocking buffer data is absent, each column value is divided by the average intensity value for the column. In an embodiment applying the CPR process, the intensity-normalized matrix is given by

$${A}_{I} ( i , j ) = \frac{{A}_{N} ( i , j )}{I ( k )} 1 \leq i , j \leq m + 1$$

where I is a vector containing the blocking buffer or average intensities and k=j if normalization is done with respect to columns.

Setting threshold values prior to row or column normalization. To prevent artificial inflation of low signal values in this normalization step, all blocking buffer values that are below a minimum user-defined threshold value are flagged and then adjusted to the user-defined threshold value which represents the lowest reliable signal intensity value, prior to row or column division. This threshold is set based on a histogram of the signal intensities. This normalization step adjusts for variations in intensity from well to well.

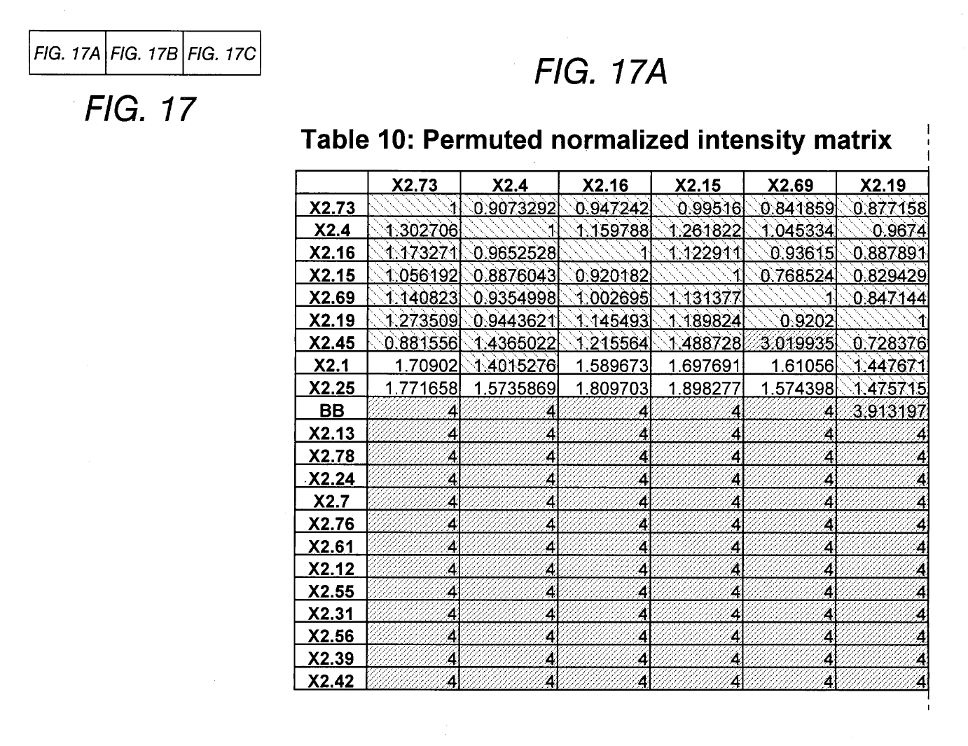

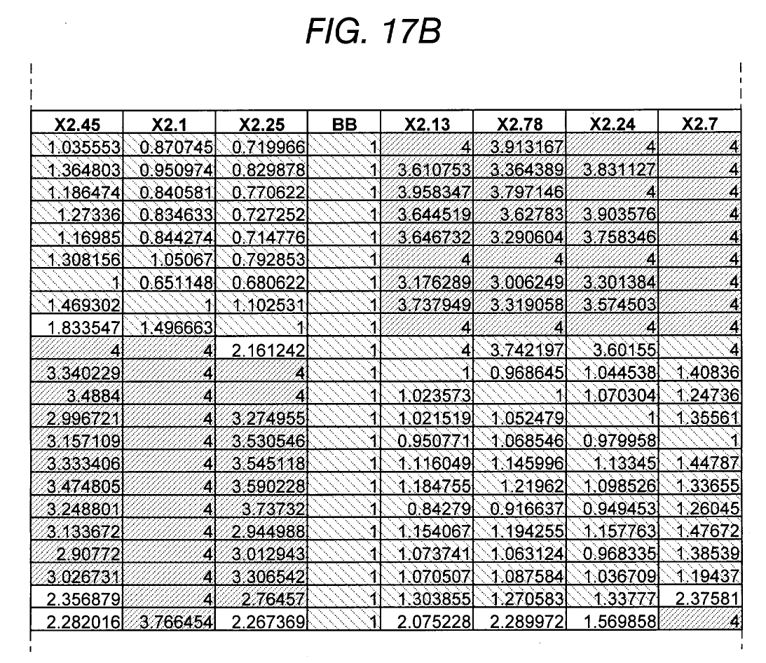

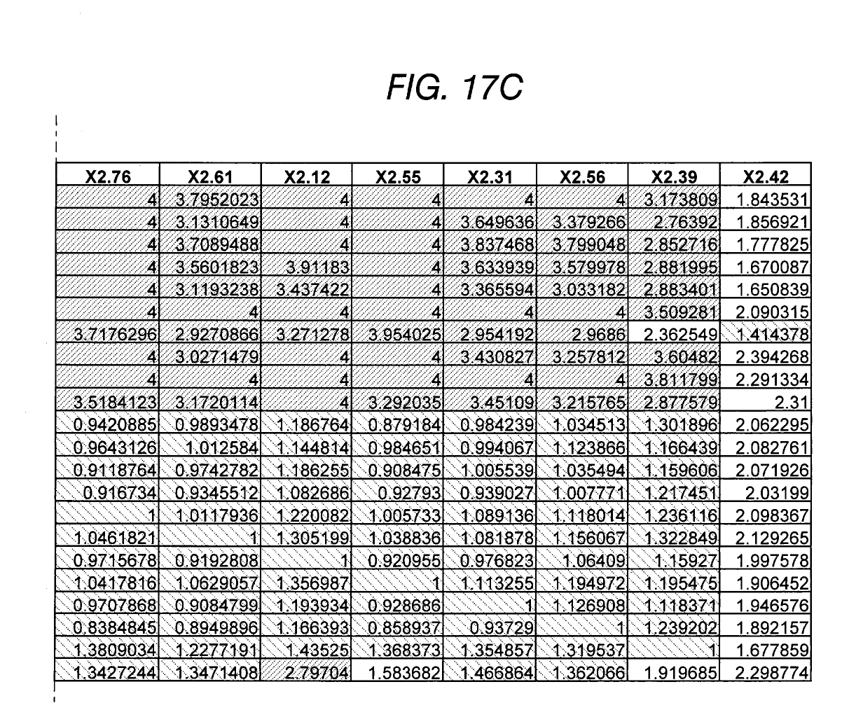

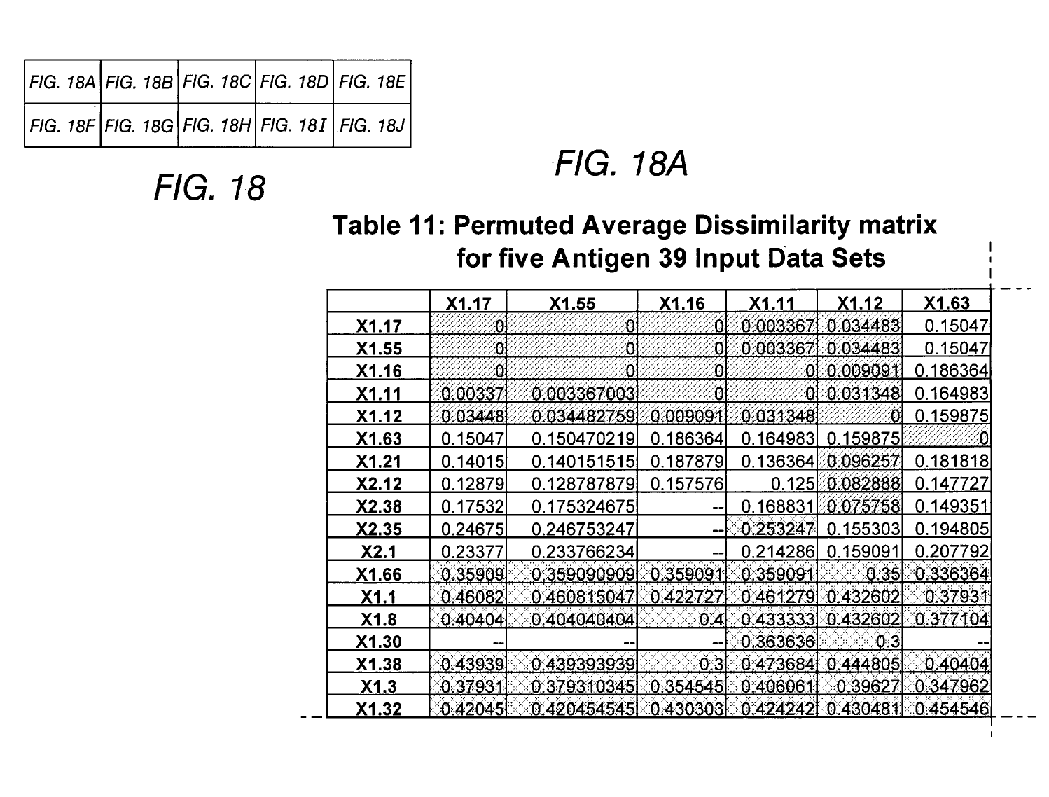

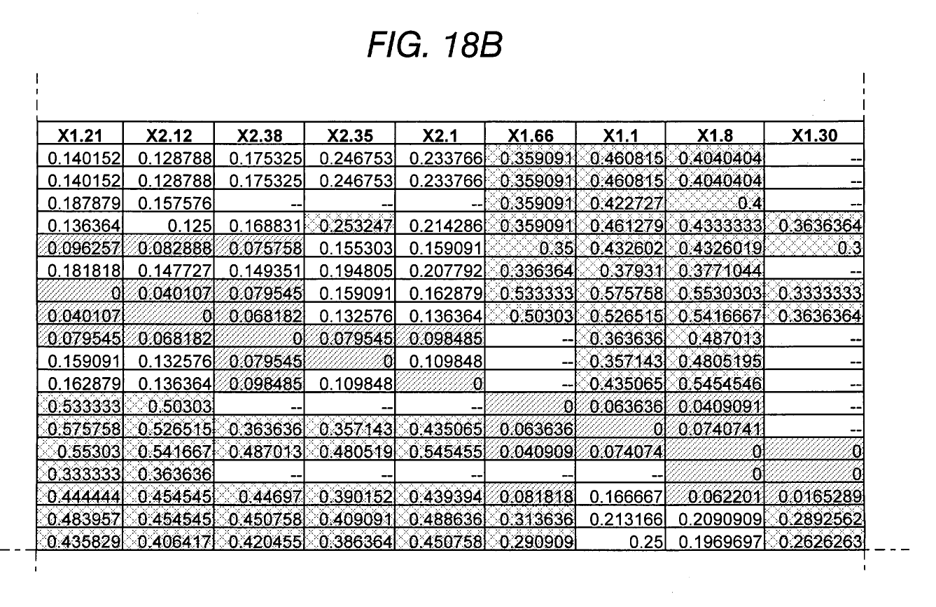

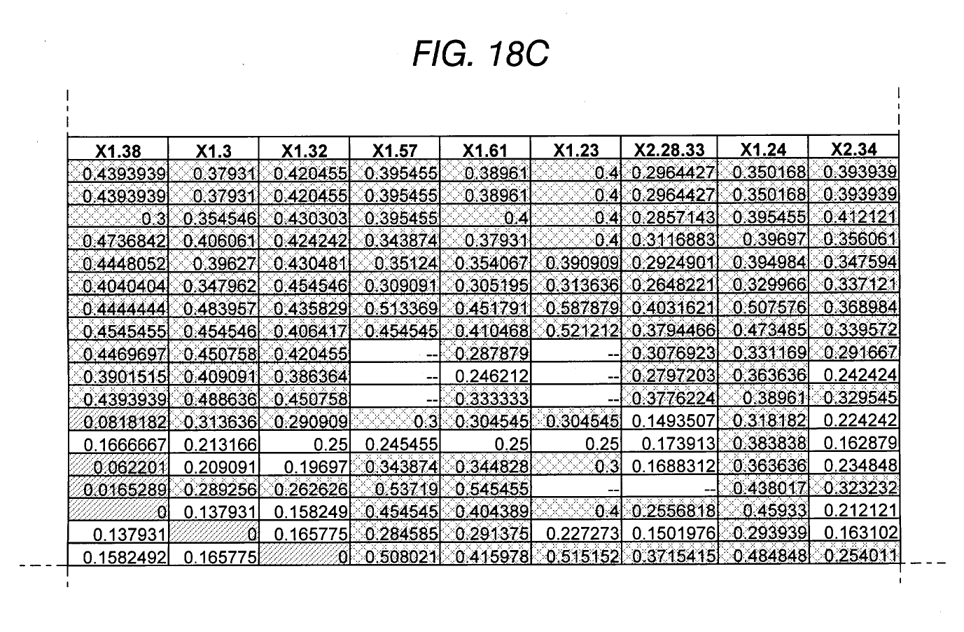

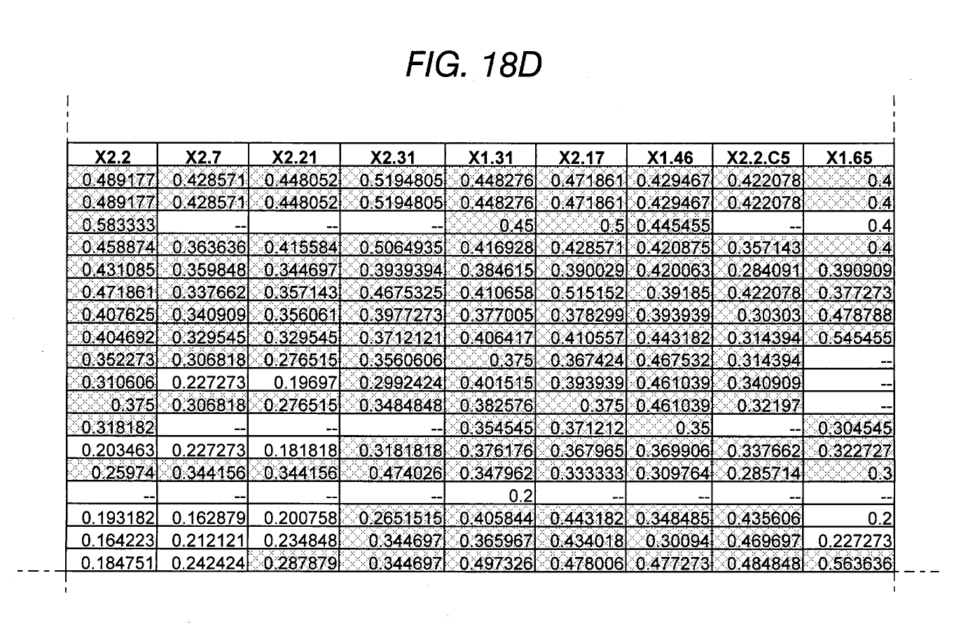

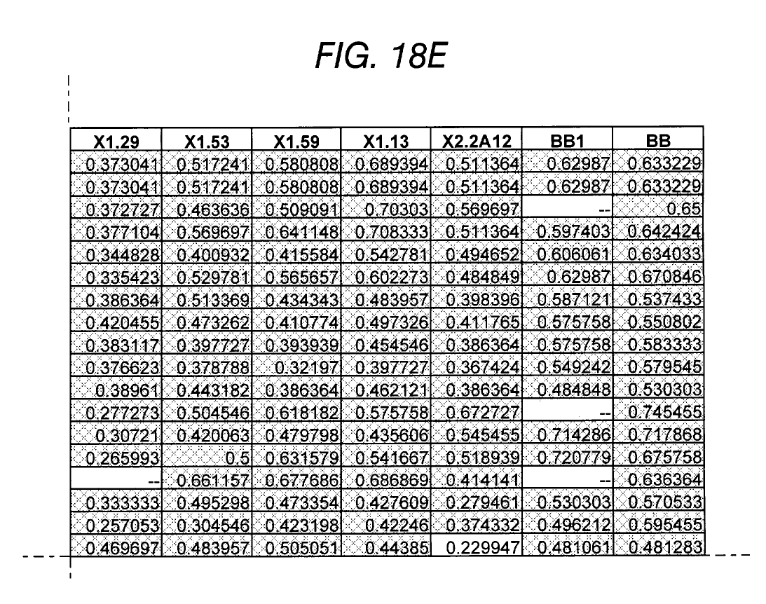

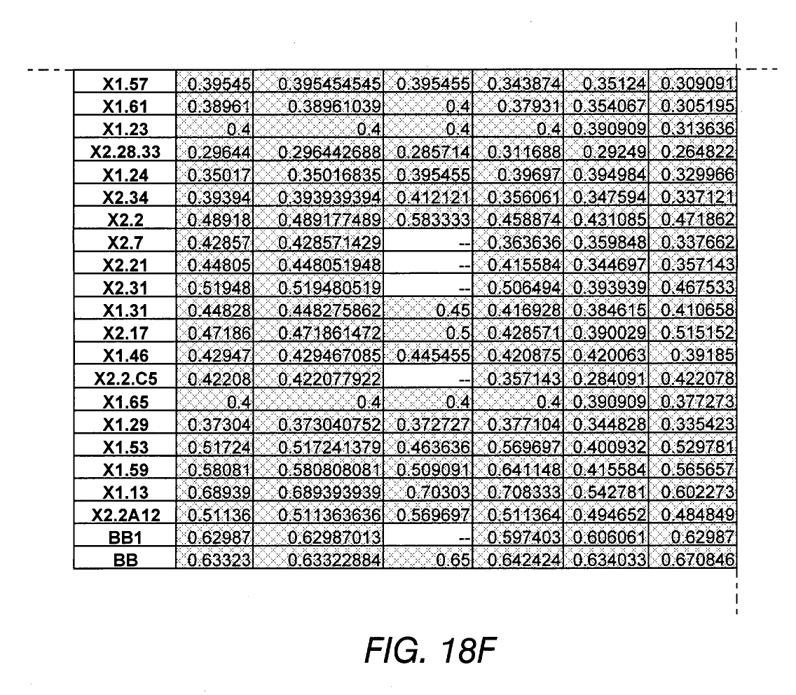

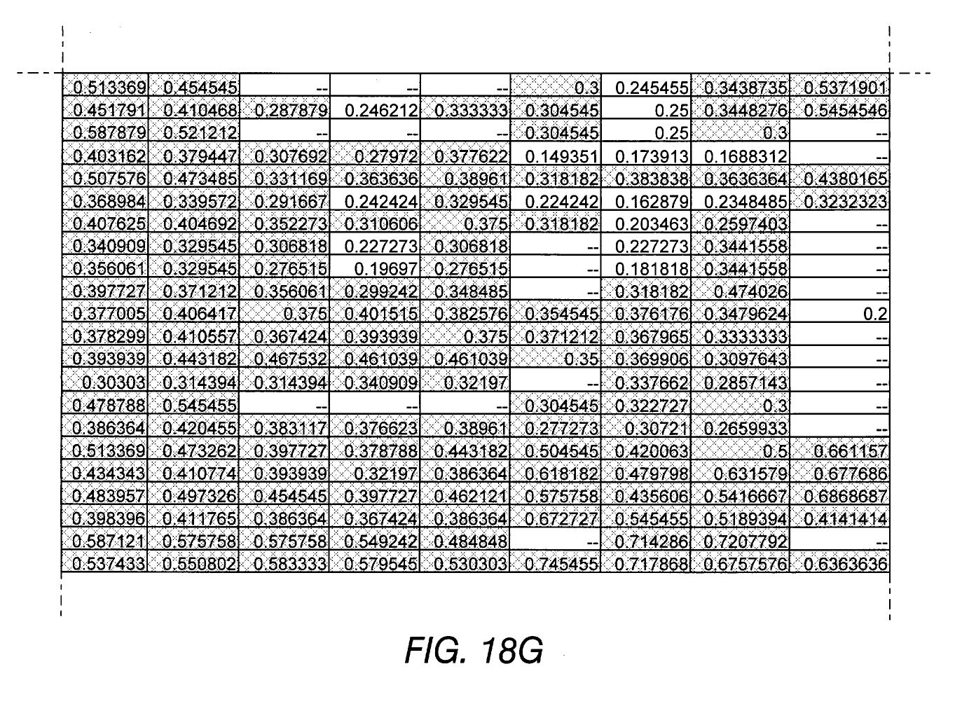

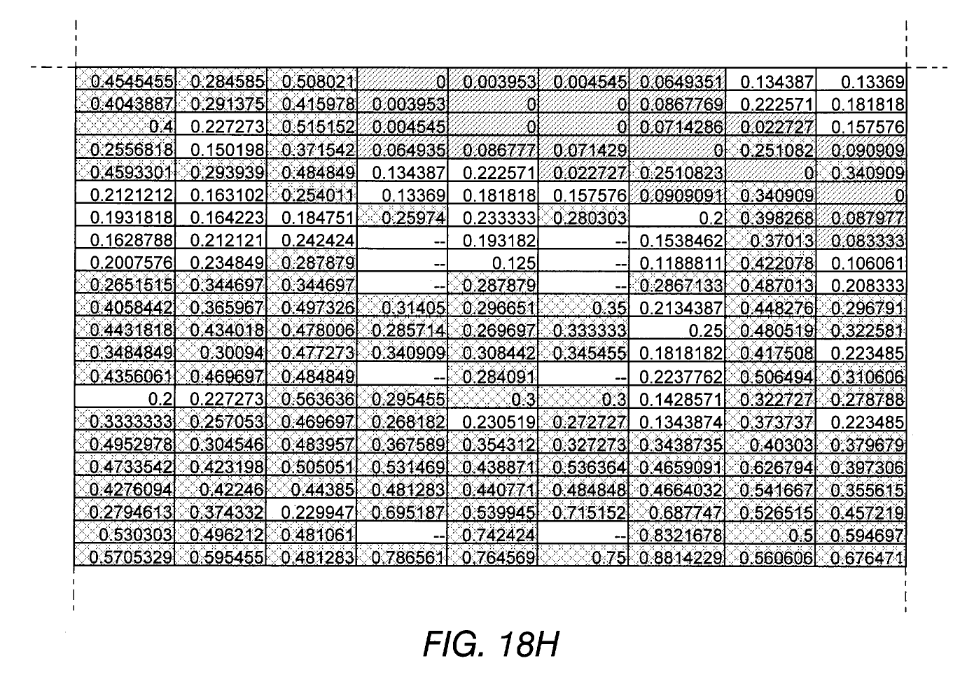

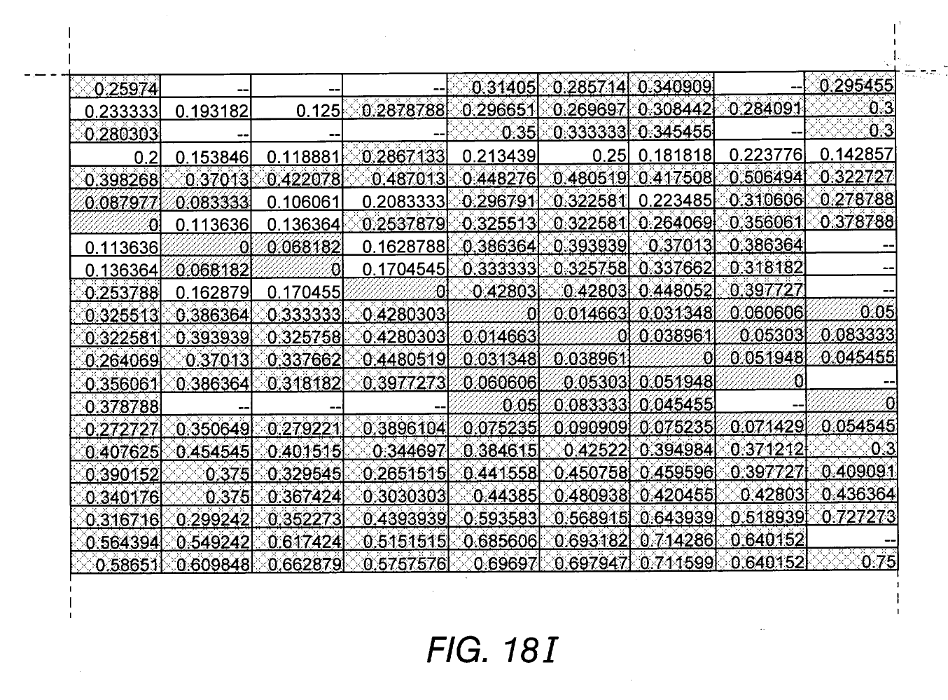

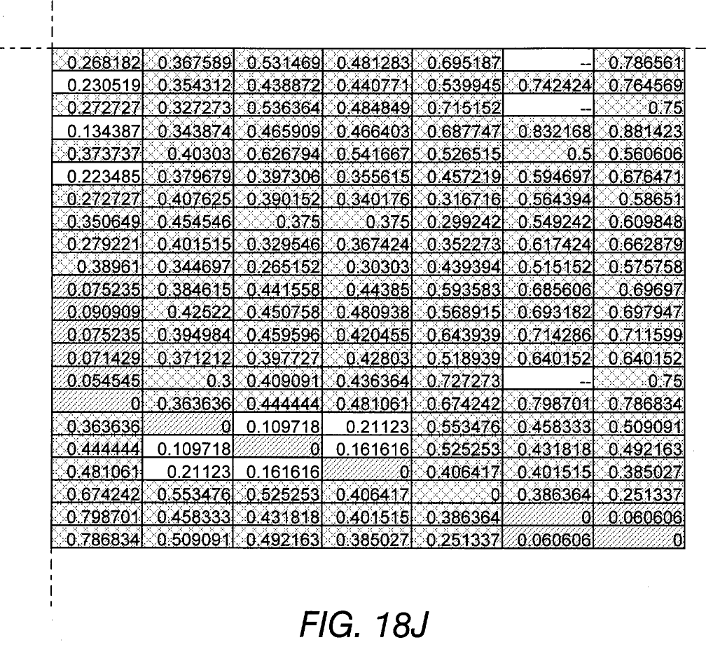

By way of example, FIG. 17 illustrates an adjusted difference matrix for the data of Example 2, wherein the minimum reliable signal intensity is set to 200 intensity units. Each row in the matrix is adjusted by dividing it by the last intensity value in the row. As noted above, the last intensity value in each row corresponds to the intensity value for beads to which blocking buffer is added in place of primary antibody. This step adjusts for the well-to-well variation in intensity values across the row. FIG. 18 illustrates a row normalized matrix for the data of Example 2.

Further by way of example, FIG. 2A presents data from an embodiment in which the blocking buffer intensity value for each row was plotted against the average intensity value for the corresponding row. This plot shows a linear correlation between the blocking buffer values and the average intensity value for a row, and suggests that there are well-specific intensity biases. These biases may be partially due to the variation in affinity for the probe antibodies for the antigen and the concentration variations of the probe antibodies. FIG. 2B presents data from an embodiment in which the blocking buffer intensity value for each column was plotted against the average intensity value for the corresponding column.

In another illustrative embodiment, FIG. 2C shows a scatter plot of the background-normalized difference matrix intensities plotted against the intensities for the matrix of results from an embodiment using antigen. This plot shows a tight linear correlation (slope=1) for signal values greater than 1000, and a more scattered correlation for lower signal values. The points in FIG. 2C are shaded according to the value of a fraction calculated as the subtracted signal divided by the signal for the experiment with antigen present. Smaller fraction values (closer to zero) correspond to high background contribution and have light shading in FIG. 2C. Larger fraction values (closer to 1) correspond to lower background contribution and have darker shading. In FIG. 2C, the smaller fraction values are predominantly in the lower-left region of the scatter plot, suggesting that the contribution of background becomes less for subtracted signal values greater than 1000.

The plot shown in FIG. 2C suggests that for this embodiment, intensity values of the background-normalized matrix greater than 1000 have a low background signal contribution relative to the signal due to antigen binding. These matrix cells likely correspond to antibody pairs that do not compete for the same epitope. Conversely, intensity values below 1000 likely correspond to antibody pairs that bind to the same epitope. In accordance with one aspect of the present invention, it is expected that the intensity values along the diagonal would be small, as identical reference and probe antibodies compete for the same epitope. In the embodiment illustrated in FIG. 2C, all but one of the diagonal values of the background-normalized signal intensity matrix have intensity values below 1000.

### Normalization of Signal Intensities Relative to the Baseline Signal for Probe Antibodies

In a final step, data are adjusted by dividing each column or row by its corresponding diagonal value to generate the final normalized matrix given by

$${A}_{F} ( i , j ) = \frac{{A}_{I} ( i , j )}{{A}_{I} ( j , j )} .$$

Once again, to prevent artificial inflation of low signal values in this normalization step, all diagonal values below a minimum user-defined threshold value are adjusted to the threshold value before the diagonal division is done. This step is done for all columns or rows, except those that have diagonal values that are significantly high relative to other values in the column or row. This step normalizes each intensity value relative to the intensity corresponding to the individual competition assay for which the reference and probe antibodies are the same. This intensity value should be low and ideally reflect the baseline signal intensity value for the column or row, because two identical antibodies should compete for the same epitope and hence be unable to simultaneously bind to the same antigen. Columns having unusually large diagonal values are identified as outliers and excluded from the analysis. High-diagonal-intensity values may indicate that the antigen has two copies of the same epitope, e.g., when the antigen is a homodimer.

### Pattern Recognition Analysis: Dissimilarity Matrices

In accordance with another aspect of the present invention, a second step in data analysis involves generating a dissimilarity matrix from the normalized intensity matrix in two steps. First, the normalized intensity values that are below a user-defined threshold value for background are set to zero (and hence represent competition) and the remaining values are set to 1, indicating that the antibodies bind to two different epitopes. Accordingly, intensity values that are less than the intensity equal to this threshold multiplied by the intensity value of the diagonal value are considered low enough to represent competition for the same epitope by the antibody pair. The dissimilarity matrix or distance matrix for a given threshold value is computed from the matrix of zeroes and ones by determining the number of positions in which each pair of rows differs. The entry in row i and column j, corresponds to the fraction of the total number of primary antibodies that differ in their competition patterns with the secondary antibodies represented in rows i and j.

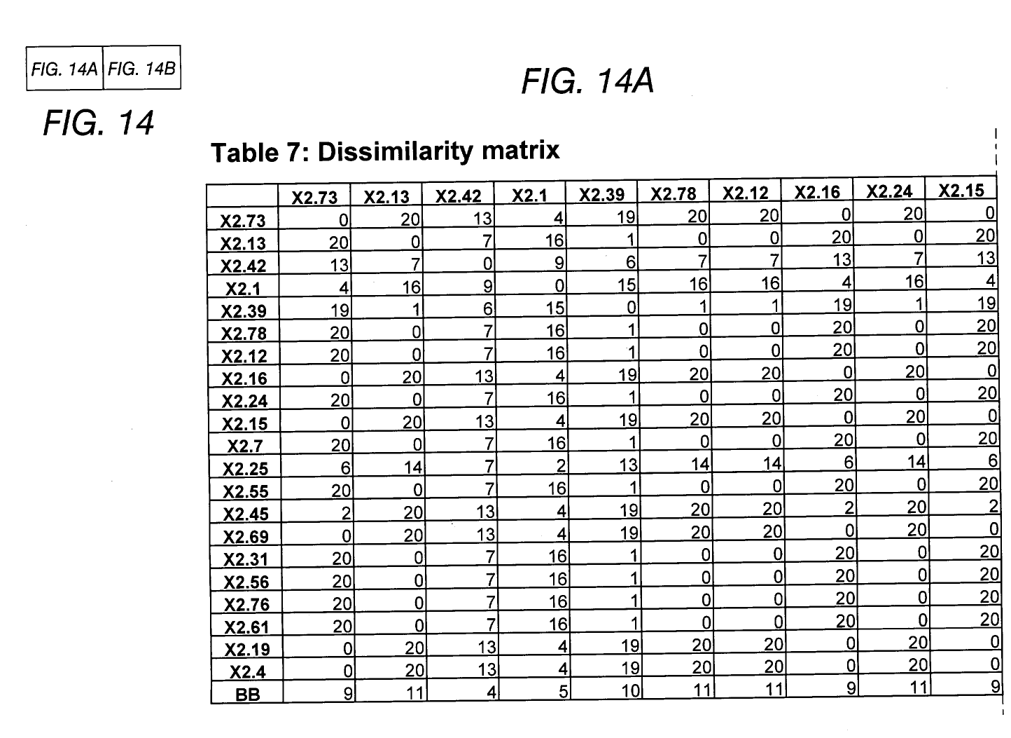

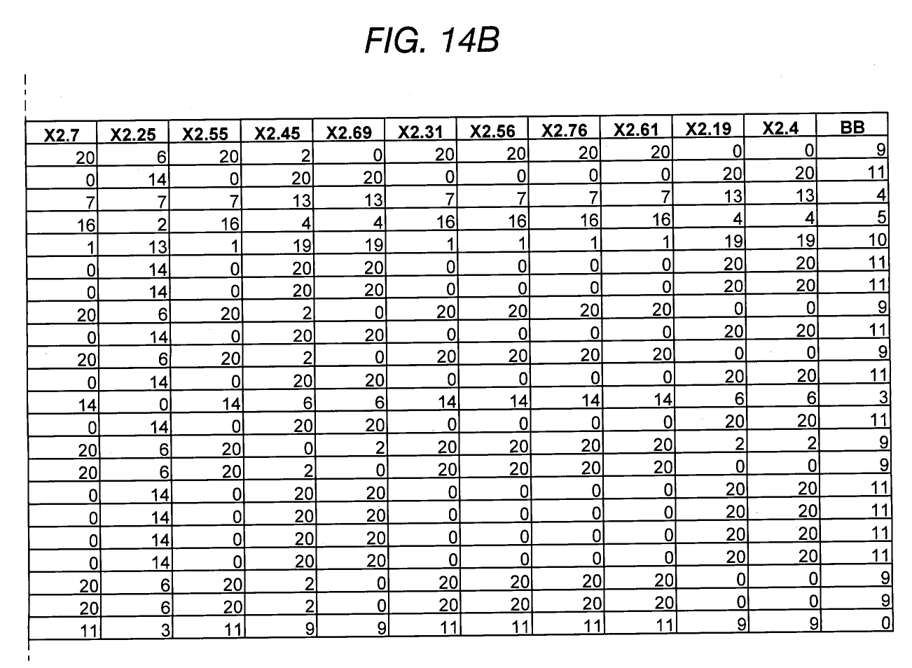

By way of example, FIG. 14 shows the number of positions (out of 22 total) at which the patterns for any two antibodies differ. In this embodiment, dissimilarities are computed with respect to rows instead of columns because the row intensities have already been adjusted for well-specific intensity biases and therefore the undesirable effects of unequal secondary antibody affinities and concentrations have been factored out. In addition, the concentrations and affinities of primary antibodies are consistent between rows. However, for the columns, there is not an apparent consistent trend between average intensity and background intensity which suggests that there is not an obvious way to factor out the undesirable affects of the variable primary antibody concentrations and affinities. Therefore, comparing the signals between columns might be less valid.

Dissimilarity matrix using CPR. In an embodiment applying the CPR process, a threshold matrix, A_{T}, of zeros and ones is generated as described below. Normalized values that are less than or equal to a threshold value are set to zero to indicate that the corresponding pairs of antibodies compete for the same epitope. The threshold matrix is given by

$${A}_{T} ( i , j ) = \{ \begin{matrix}0 & if & {A}_{F} ( i , j ) \leq T \\ 1 & if & {A}_{F} ( i , j ) > T .\end{matrix}$$

The remaining normalized intensity values are set to one, and the values represent pairs of antibodies that bind to different epitopes.

The dissimilarity matrix is computed from the threshold matrix by setting the value in the i^{th} row and j^{th} column of the dissimilarity matrix to the fraction of the positions at which two rows, i and j of the matrix of zeros and ones, differ. A dissimilarity matrix for a specified threshold value, T, is given by

$${D}_{T} ( i , j ) = \frac{m - {N}_{1} ( i , j )}{m}$$

where N₁ is the number of 1's present when the i^{th} and j^{th} rows are summed.

By way of example, for the matrix shown in Table 1 below, the dissimilarity value corresponding to the first and second rows is 0.4, because the number of positions at which the two rows differ is 2 out of 5. For an ideal experiment, the dissimilarity matrix that is generated based on a comparison of rows of the original signal intensity matrix, should be the same as the dissimilarity matrix that is generated based on the comparison of columns.

TABLE 1
Matrix Used to Compute Dissimilarity Values

| | A | B | C | D | E |
|---|---|---|---|---|---|
| A | 0 | 1 | 1 | 1 | 0 |
| B | 1 | 1 | 1 | 0 | 0 |
| C | 1 | 1 | 1 | 1 | 1 |
| D | 1 | 1 | 1 | 0 | 1 |
| E | 1 | 0 | 1 | 1 | 0 |

Effect of calculating dissimilarity matrices at multiple threshold values.

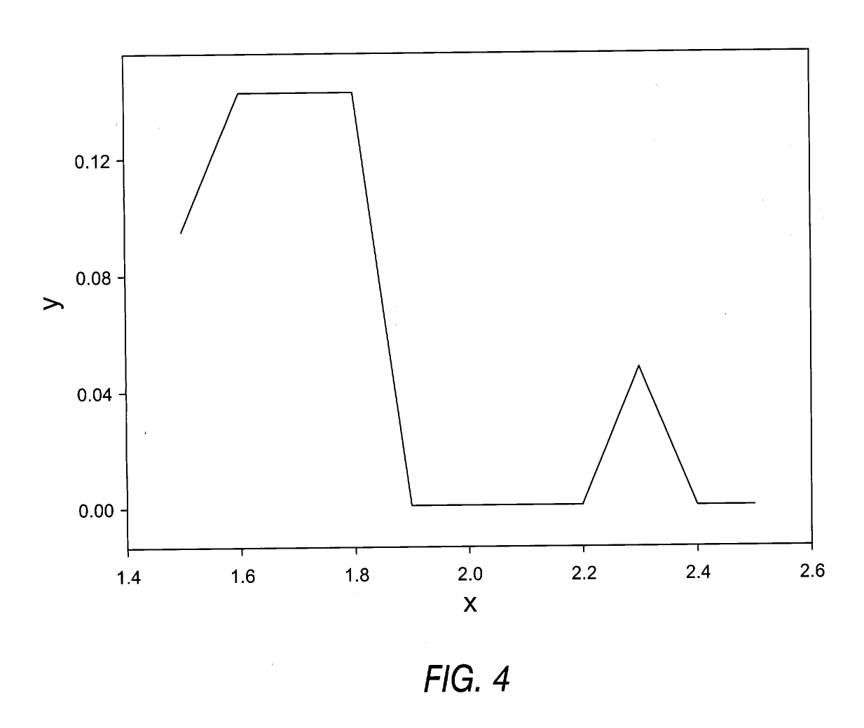

If desired, the process of generating dissimilarity matrices is repeated for background threshold values incremented inclusively between two user-defined threshold values which represent lower and upper threshold values for intensity (where the threshold value is as described above) The dissimilarity matrices generated over a range of background threshold values is averaged and used an input to the clustering algorithm. The process of averaging over several thresholds is performed to minimize the sensitivity of the final dissimilarity matrix to any one particular choice for the threshold value. The effect of variation of the threshold value on the apparent dissimilarity is illustrated by FIG. 4, which shows the fraction of dissimilarities for a pair of antibodies (2.1 and 2.25) as a function of the threshold value for threshold values ranging between 1.5 and 2.5. As the threshold value changes from 1.8 to 1.9 the amount of dissimilarity between the signal patterns for the two antibodies changes substantially from 15% to nearly 0%. This figure shows how the amount of dissimilarity between the signal patterns for a pair of antibodies may be sensitive to one particular choice for a cutoff value, as it can vary substantially for different threshold values. The sensitivity is mitigated by taking the average dissimilarity value over a range of different threshold values.

Calculating dissimilarity matrices at multiple threshold values using CPR. In a preferred embodiment, the process of computing dissimilarity matrices using CPR is repeated for several incremental threshold values within a user-defined range of values. The average of these dissimilarity matrices is computed and used as input to the clustering step where the average is computed as

$${D}_{Ave} ( i , j ) = \frac{{\sum }_{T} {D}_{T} ( i , j )}{{N}_{T}}$$

where N_{T} is the number of different thresholds to be averaged.

This process of averaging over several thresholds is done to minimize the sensitivity of the dissimilarity matrix to a particular cutoff value for the threshold.

### Dissimilarity Matrices From Multiple Experiments

If there are input data sets for more than one experiment, normalized intensity matrices are first generated as described above for each individual experiment. Normalized values above a threshold value (typically set to 4) are then set to this threshold value. Setting the high-intensity values to the threshold value is done to prevent any single intensity value from having too much weight when the average normalized intensity values are computed for that cell. The average intensity matrix is computed by taking individual averages over all data points for each antibody pair out the group consisting of antibodies that are in at least one of the input data sets. Antibody pairs for which there are no intensity values are flagged. The generation of the dissimilarity matrix is as described above with the exception that the entry in row i and column j corresponds to the fraction of the positions at which two rows, i and j differ out of the total number of positions for which both rows have an intensity value. If the two rows have no such positions, then the dissimilarity value is set arbitrarily high and flagged.

### Clustering of Antibodies Based on Their Normalized Signal Intensities

Another aspect of the present invention provides processes for clustering antibodies based on their normalized signal intensities, using various computational approaches to identify underlying patterns in complex data. Preferably, any such process utilizes computational approaches developed for clustering points in multidimensional space. These processes can be directly applied to experimental data to determine epitope binding patterns of sets of antibodies by regarding the signal levels for the n² competition assays of n probe antibodies in n sampled reference antibodies as defining n points in n-dimensional space. These methods can be directly applied to epitope binning by regarding the signal levels for the competition assays of each secondary antibody with all of the n different primary antibodies as defining a point in n-dimensional space.

Results of clustering analysis can be expressed using visual displays. In addition or in the alternative, the results of clustering analysis can be captured and stored independently of any visual display. Visual displays are useful for communicating the results of an epitope binning assay to at least one person. Visual displays may also be used as a means for providing quantitative data for capture and storage. In one preferred embodiment, clusters are displayed in a matrix format and information regarding clusters is captured from a matrix. Cells of a matrix can have different intensities of shading or patterning to indicate the numerical value of each cell; alternately, cells of a matrix can be color-coded to indicate the numerical value of each cell. In another preferred embodiment, clusters are displayed as dendrograms or “trees” and information regarding clusters is captured from a dendrogram based on branch length and height (distance) of branches. In yet another preferred embodiment, clusters are identified by automated means, and information regarding clusters is captured by an automated data analysis process using a computer or any data input device.

One approach that has proven valuable for the analysis of large biological data sets is hierarchical clustering (Eisen et al. (1998) Proc. Natl. Acad. Sci. USA 95:14863-14868). Applying this method, antibodies can be forced into a strict hierarchy of nested subsets based on their dissimilarity values. In an illustrative embodiment, the pair of antibodies with the lowest dissimilarity value is grouped together first. The pair or cluster(s) of antibodies with the next smallest dissimilarity (or average dissimilarity) value is grouped together next. This process is iteratively repeated until one cluster remains. In this manner, the antibodies are grouped according to how similar their competition patterns are, compared with the other antibodies. In one embodiment, antibodies are grouped into a dendrogram (sometimes called a “phylogenetic tree”) whose branch lengths represent the degree of similarity between the binding patterns of the two antibodies. Long branch lengths between two antibodies indicate they likely bind to different epitopes. Short branch lengths indicate that two antibodies likely compete for the same epitope.

In a preferred embodiment, the antibodies corresponding to the rows in the matrix are clustered by hierarchical clustering based on the values in the average dissimilarity matrix using an agglomerative nesting subroutine incorporating the Manhattan metric with an input dissimilarity matrix of the average dissimilarity matrix. In an especially preferred embodiment, antibodies are clustered by hierarchical clustering based on the values in the average dissimilarity matrix using the SPLUS 2000 agglomerative nesting subroutine using the Manhattan metric with an input dissimilarity matrix of the average dissimilarity matrix. (SPLUS 2000 Statistical Analysis Software, Insightful Corporation, Seattle, Wash.)

In accordance with another aspect of the present invention, the degree of similarity between two dendrograms provides a measure of the self-consistency of the analyses performed by a program applying the CPR process. A non-limiting theory regarding similarity and consistency predicts that a dendrogram generated by clustering rows and a dendrogram generated by clustering columns of the same background-normalized signal intensity matrix should be identical, or nearly so, because: if Antibody #1 and Antibody #2 compete for the same epitope, then the intensity should be low when Antibody #1 is the reference antibody and Antibody #2 is the probe antibody, as well as when Antibody #2 is the reference antibody and Antibody #1 is the probe antibody. Likewise, when the two antibodies bind to different epitopes, the intensities should be uniformly high. By this reasoning, the degree of similarity between two rows of the signal intensity matrix should be the same as between two columns of the similarity matrix. A high level of self-consistency between row clustering and column clustering suggests that, for a given experiment, the experimental protocol described herein, practiced with the program for applying the process of the present invention, produces robust results.

In accordance with a further aspect of the present invention, the degree of overlap between two epitopes may also be inferred based on the lengths of the longest branches connecting clusters in a dendrogram. For example, if a target antigen has two distinct, completely nonoverlapping epitopes, then one would expect that an antibody binding to one of the epitopes would have an opposite signal intensity pattern from an antibody binding to another epitope. According to this reasoning, if the binding sites are nonoverlapping, the signal patterns for the set of antibodies binding one epitope should be completely anticorrelated to the signal pattern for the set of antibodies recognizing the other epitope. Hence, dissimilarity values that are close to one (1) for two different clusters suggest that the corresponding epitopes do not interfere with each other or overlap in their binding sites on the antigen.

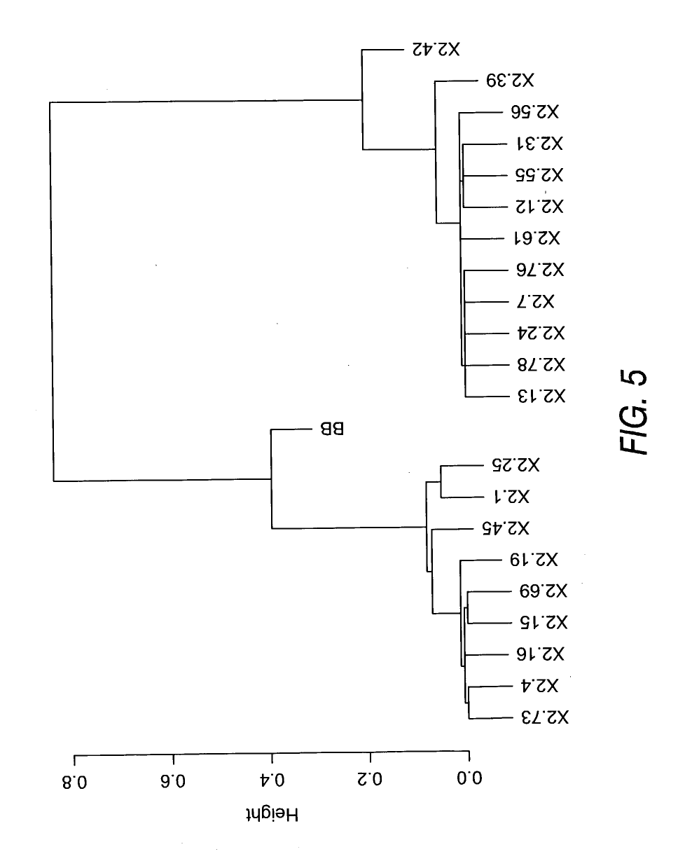

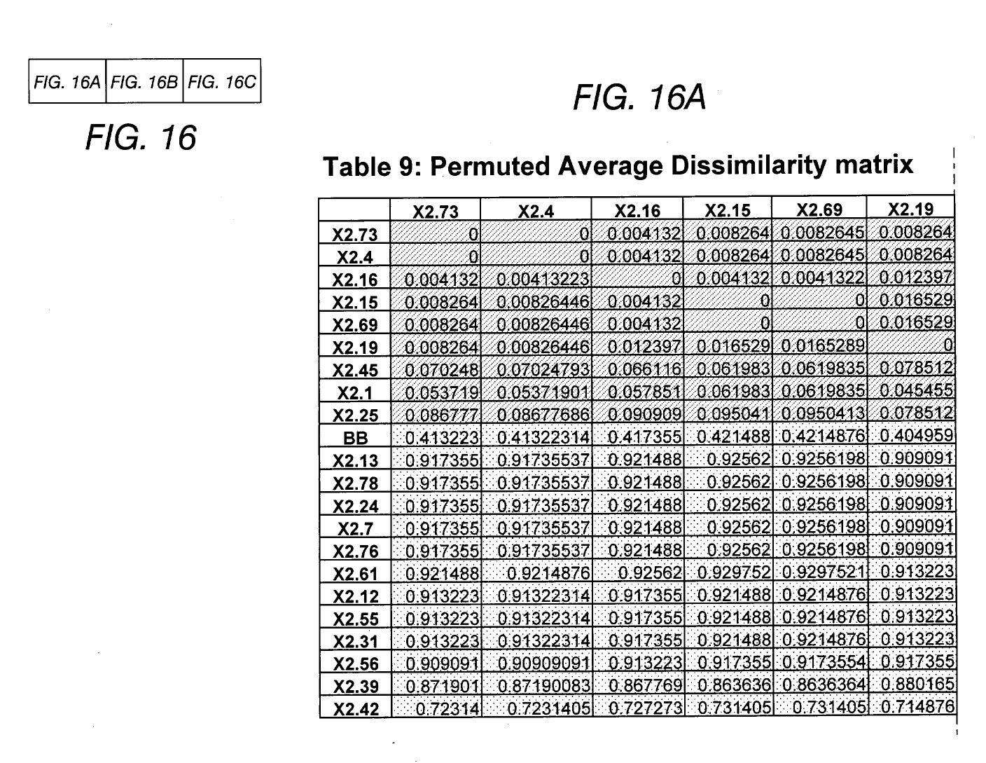

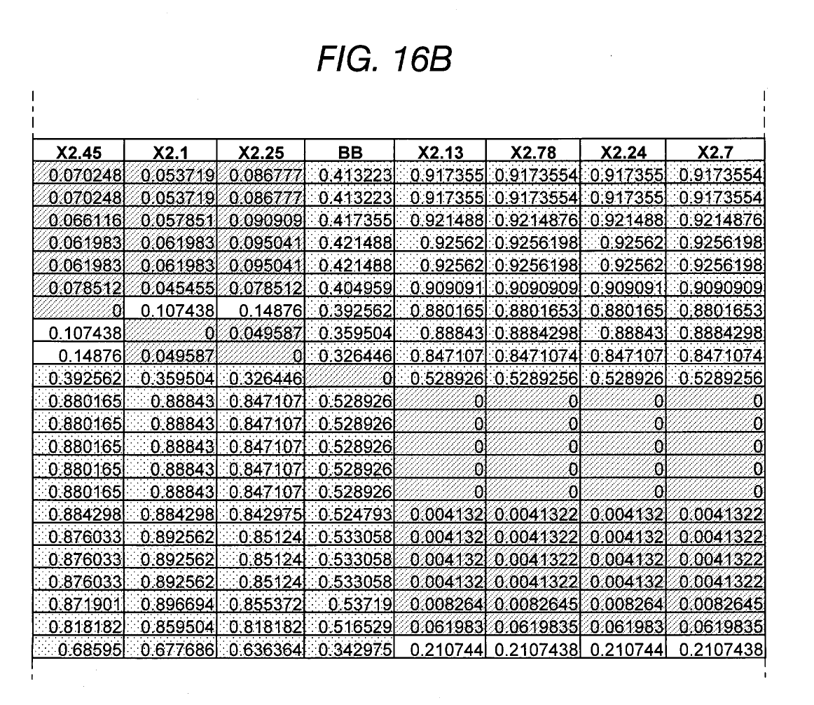

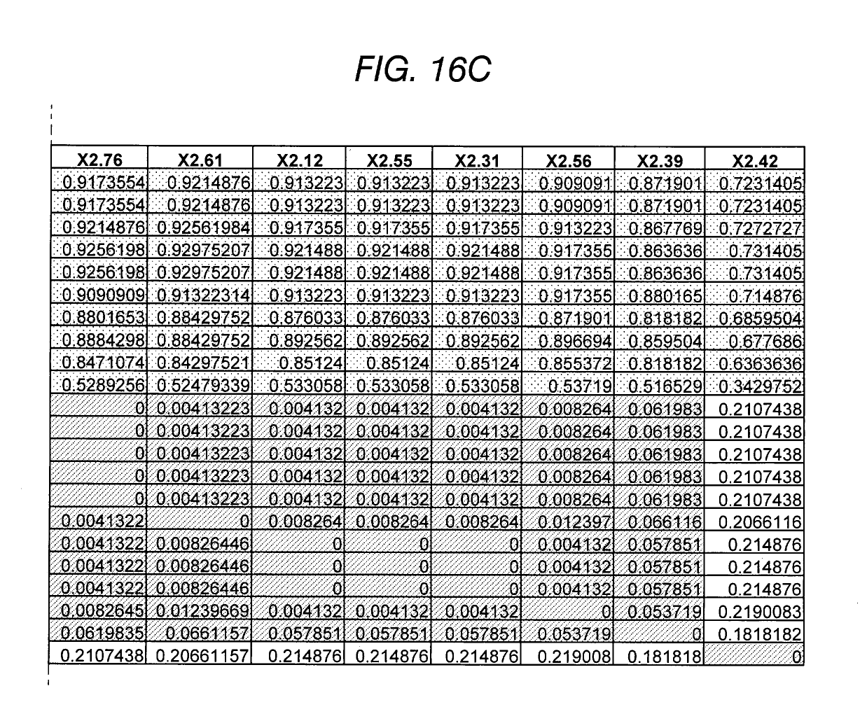

The embodiment described in Example 2 below demonstrates how clustering results can be displayed as a dendrogram (FIG. 5) or in matrix form (FIGS. 16 and 17). The data points (values of antibodies against the ANTIGEN14 target) were grouped into a dendrogram whose branch lengths represent the degree of similarity between two antibodies, where the dendrogram was generated using the Agglomerative Nesting module of the SPLUS 2000 statistical analysis software. To facilitate comparison, In FIG. 16 and 17, the order of the antibodies in rows and columns of the matrices is the same as the order of the antibodies as displayed from left to right under the dendrogram in FIG. 5. The individual cells are visually coded by shading cells according to their numerical value. In FIG. 16, cells with values below a lower threshold value have darker shading. Cells with values below a lower threshold and an upper threshold are unshaded. Cells with values above the upper threshold have lighter shading. A block having cells that are unshaded or have darker shading indicates that all of the antibodies corresponding to that block that recognize the same epitope. Cells with lighter shading correspond to antibodies that recognize different epitopes. In FIG. 17, the cells are the normalized intensity values and are also visually coded according to their value. Cells that have lighter shading have intensities below a lower threshold, unshaded cells have intensities between a lower and an upper threshold, while cells with darker shading have intensities above an upper threshold. A cell with lighter shading indicates the antibodies in its corresponding row and column compete for the same epitope (as the intensity is low). A darker cell corresponds to a higher intensity and is indicative that the antibodies in the corresponding row and column bind to different epitopes.

The results from this illustrative embodiment (Example 2) indicate that the processes of the present invention provide a high level of self-consistency for the data with regard to revealing whether or not two antibodies compete for the same epitope. The symmetry of the shading in FIGS. 16 and 17 with respect to the diagonal clearly shows this self-consistency. The reason is that the antibodies in row A and column B are the same pair as in row B and column A. Hence, if the pair of antibodies compete for the same epitope, then the intensity should be low both when antibody A is the primary antibody and antibody B is the secondary antibody, as well as when antibody B is the primary antibody and antibody B is the secondary antibody. Therefore, the intensity for the cell of the ith row and jth column as well that for the jth row and ith column should both be low. Likewise, if these two antibodies recognize different epitopes, then both corresponding intensities should be high. Out of the approximately 200 pairs of cells in FIG. 17, only one pair showed a discrepancy where one member of the pair had an intensity below 1.5 while the other member had an intensity above 2.5. The level of self-consistency of the resulting normalized matrices produced by the algorithm provides a measure of the reliability of both the data generated as well as the algorithm's analysis of the data. The high level of self-consistency for the data set (over 99%) of antibodies against the ANTIGEN14 target suggest that the data analysis processes disclosed and claimed herein generate reliable results.

### Clustering Antibodies From Multiple Experiments

Another aspect of the present invention provides a method for combining data sets to overcome limitations of experimental systems used to screen antibodies. By performing multiple experiments in which each experiment has at least x antibodies in common with each other experiment, and providing the multiple resulting data sets as input to the clustering process, it should be possible to reliably cluster very large numbers of antibodies. By having a set of m antibodies in common between the m experiments, it becomes possible to infer which cluster antibodies are likely to belong to even if they are not tested against every other antibody. This suggests that using this method for data analysis with multiple data sets, it may be possible to achieve an even higher throughput with fewer assays

By way of example, the Luminex technology provides 100 unique fluorochromes, so it is possible to study 100 antibodies at most in a single experiment. The consistency of results produced by the clustering step for individual data sets and the combined data set indicate that it is possible to infer which epitope is recognized by which antibody, even if the epitope and/or antibody are not tested against every other antibody. In a preferred embodiment, the CPR process can be used to characterize the binding patterns of more than 100 antibodies by performing multiple experiments using overlapping antibody sets. By designing experiments in such a way that each experiment has a set of antibodies in common with the other experiments, the combined-average matrix will not have any missing data.

A further aspect provides that the results of data analysis for a given set of antibodies are useful to aid in the rational design of subsequent experiments. For example, if a data set for a first experiment shows well-defined clusters emerging, then the set of antibodies for a second experiment should include representative antibodies from the first set of antibodies as well as untested antibodies. This approach ensures that each set of antibodies has sufficient material to define the two epitopes, and that the sets overlap sufficiently to permit comparison between sets. By comparing the competition patterns of an untested set of antibodies in the second experiment with a sample set of known antibodies from the first experiment, it should be possible to determine whether or not the untested antibodies recognize the same epitope(s) as do the first set of antibodies. This overlapping experimental design permits reliable comparison of the competition patterns of the first set with the second set of antibodies, to determine whether the antibodies in the second experiment recognize existing epitopes, or whether they recognize one or more completely novel epitopes. Further, experiments can be iteratively designed in an optimal way, so that multiple sets of antibodies can be tested against existing and new clusters.

### Analysis of Data From Multiple Experiments

Results from the embodiment described in Example 3 below, using antibodies against the ANTIGEN39 target, demonstrate that the processes disclosed and claimed herein are suitable for analyzing data from multiple experiments. In this embodiment, ANTIGEN39 antibodies were tested for binding to cell surface ANTIGEN39 antigen, where ANTIGEN39 antigen is a cell surface protein. First, normalized intensity matrices were generated for each individual experiment, wherein normalized values above a selected threshold value are set to the selected threshold value to prevent any single normalized intensity value from having too much influence on the average value for that antibody pair. A single normalized matrix was generated from the individual normalized matrices by taking the average of the normalized intensity values over all experiments for each antibody pair for which data was available. Then a single dissimilarity matrix was generated as described above, with the exception that the fraction of the positions at which two rows, i and j differ only considers the number of positions for which both rows have an intensity value.

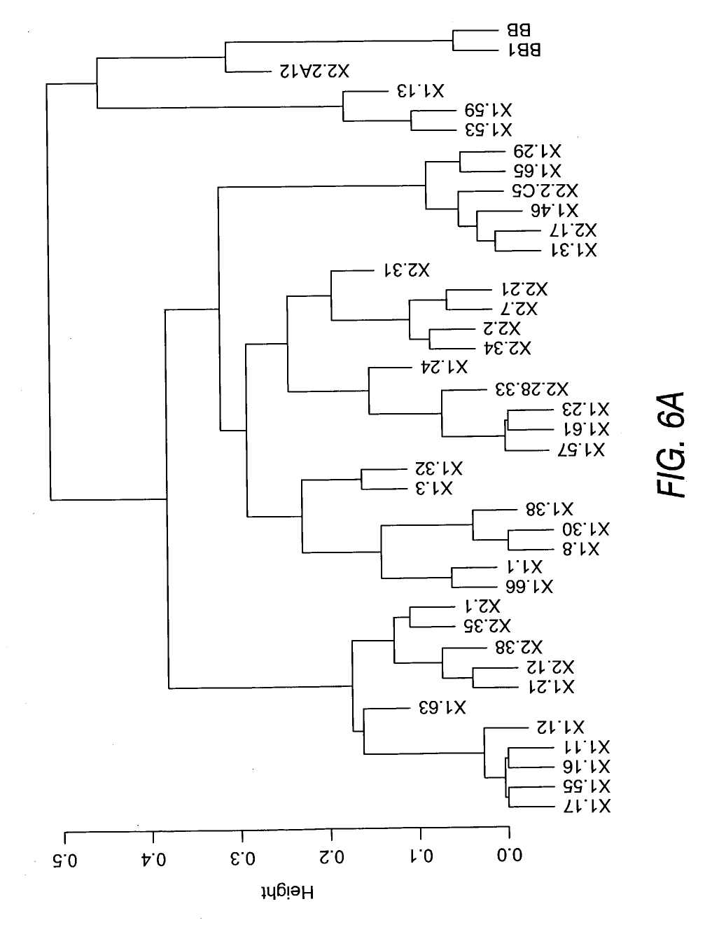

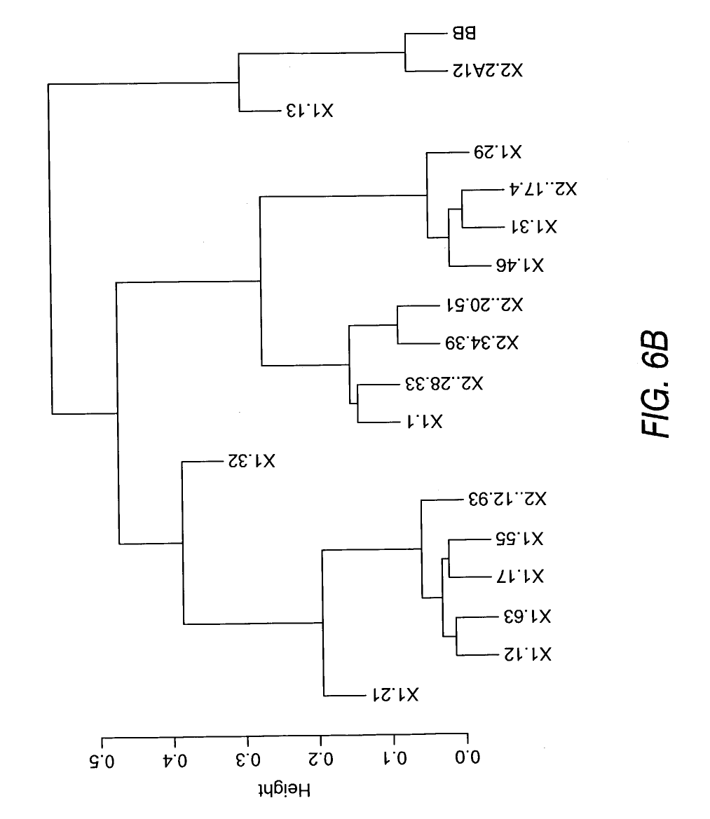

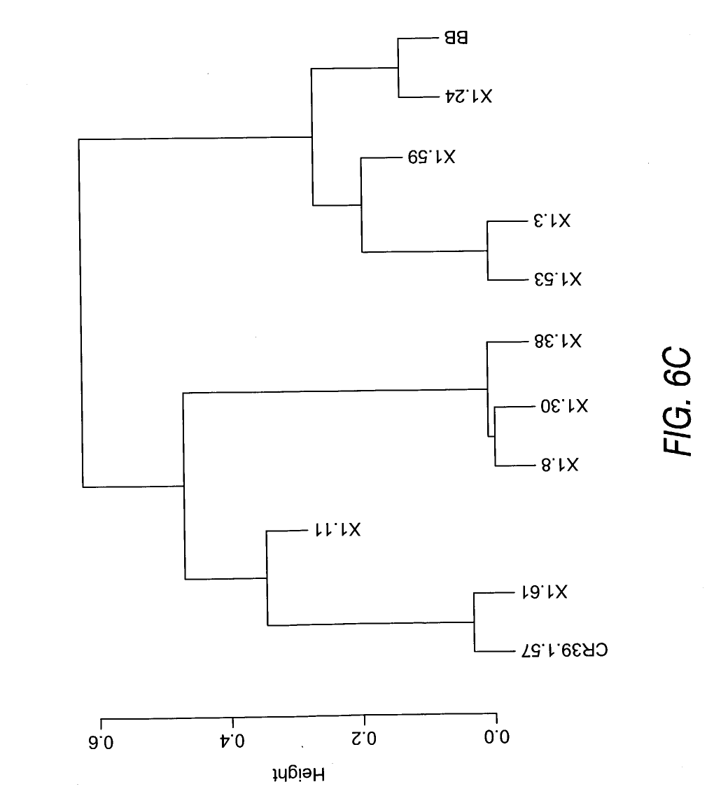

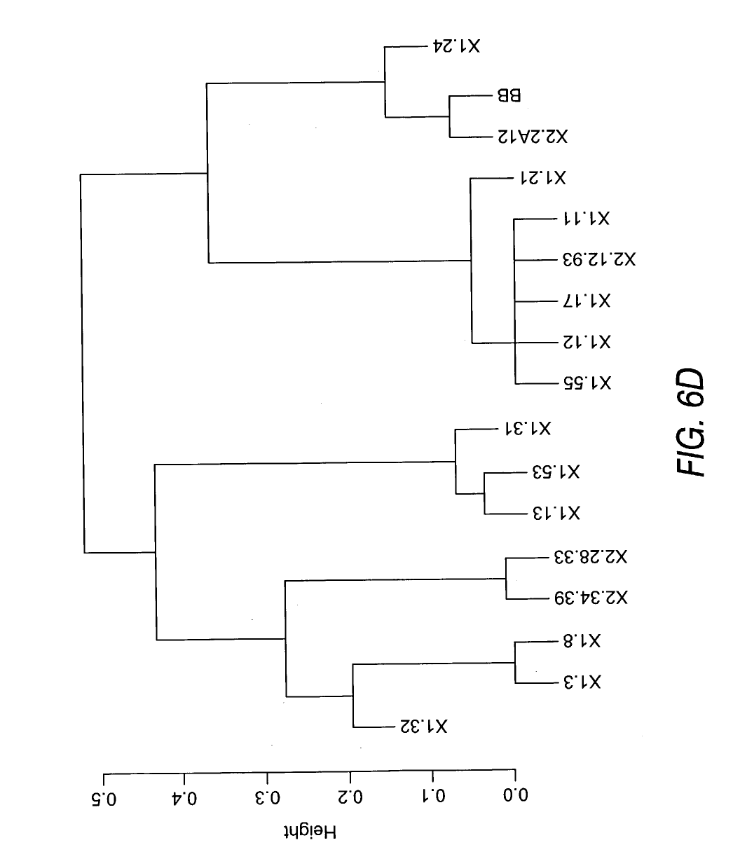

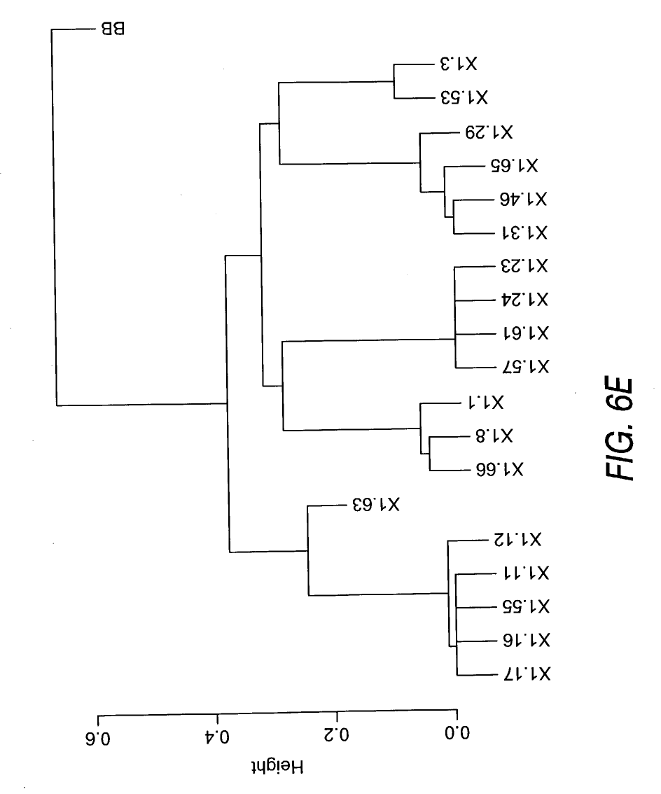

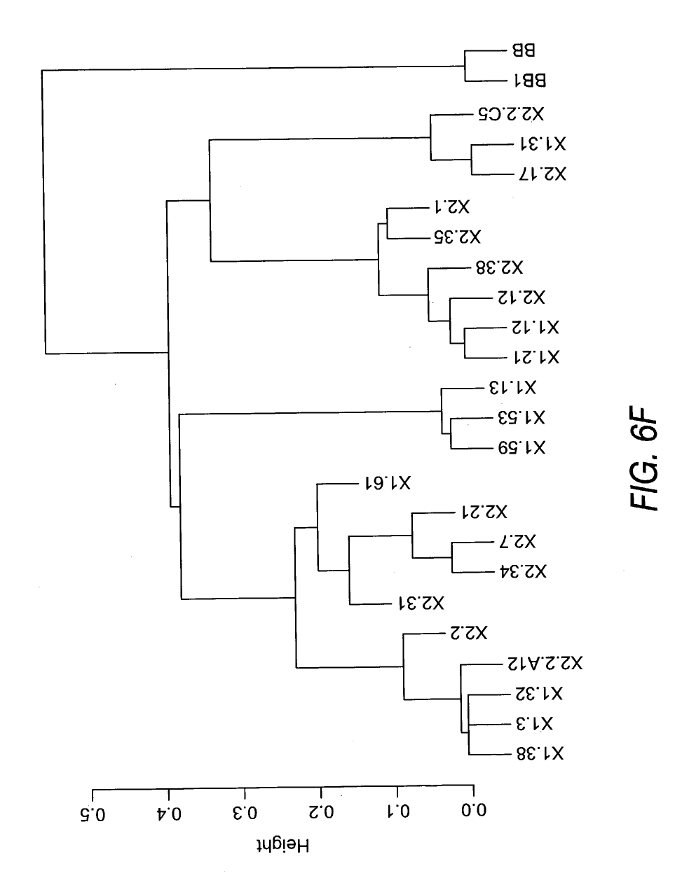

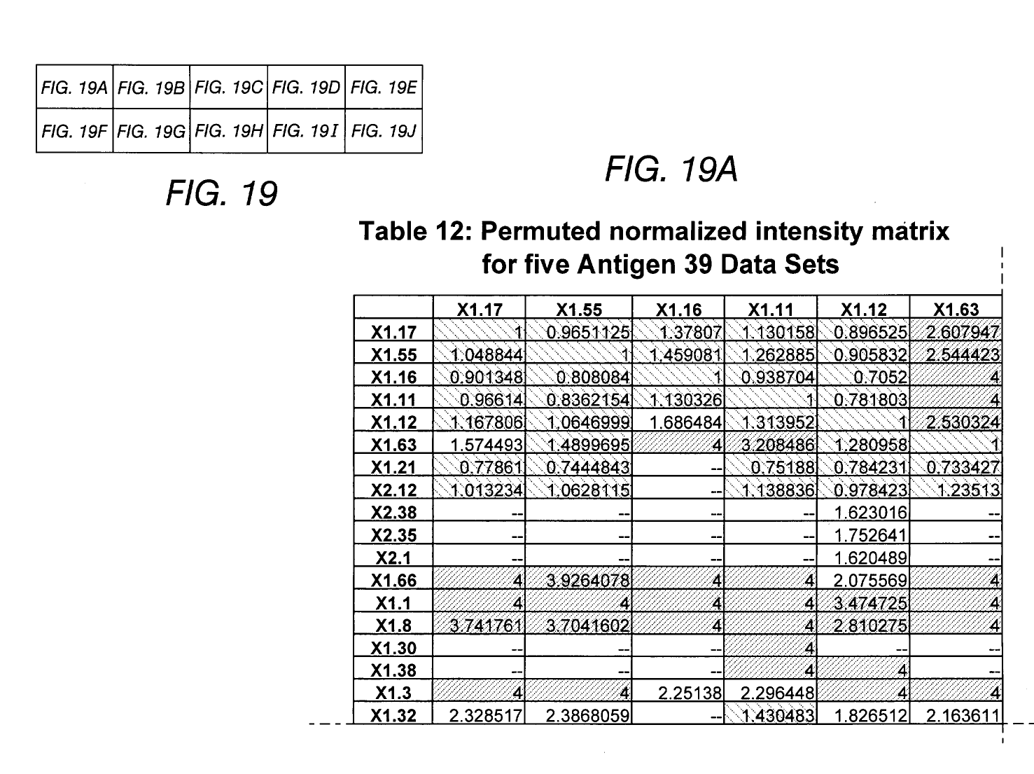

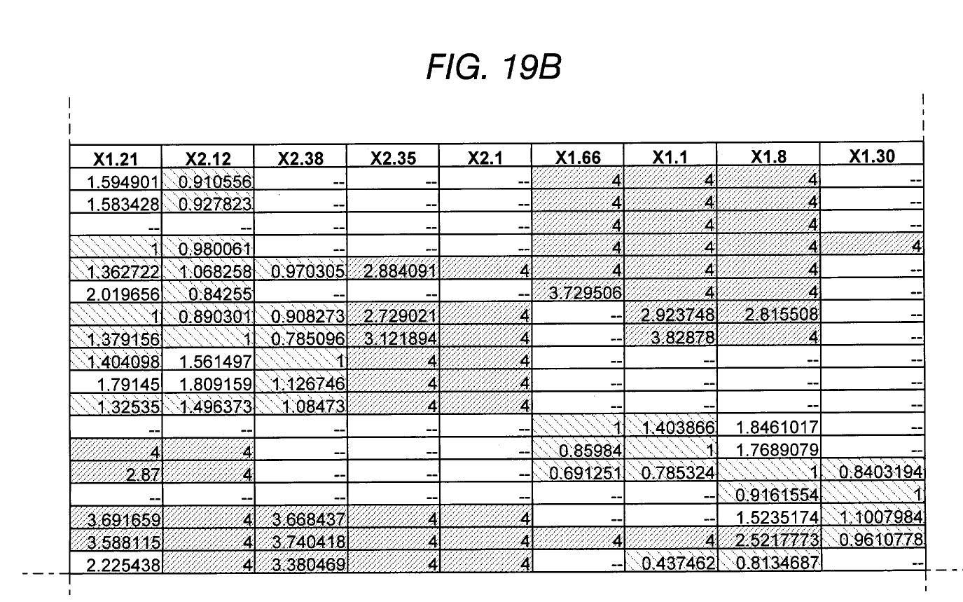

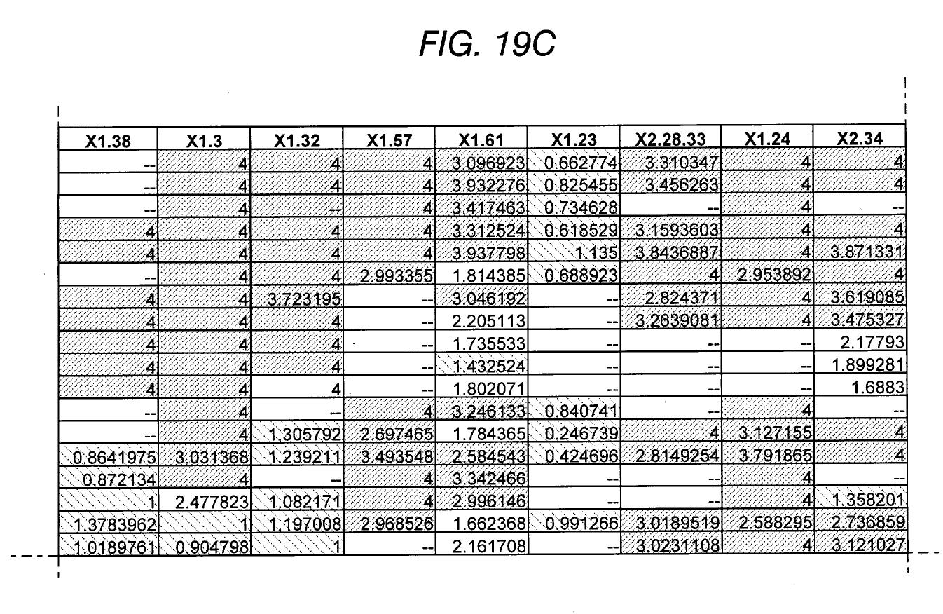

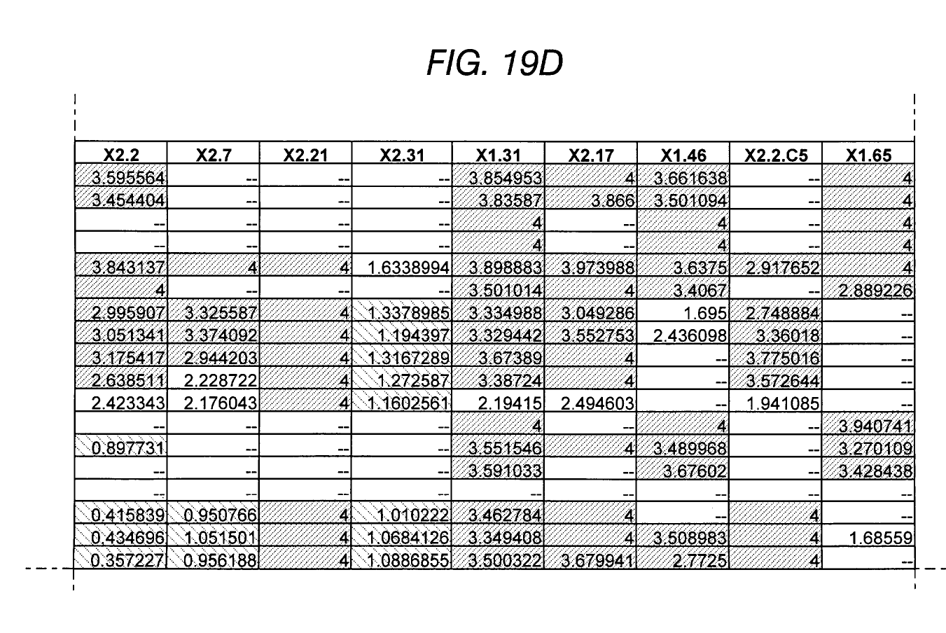

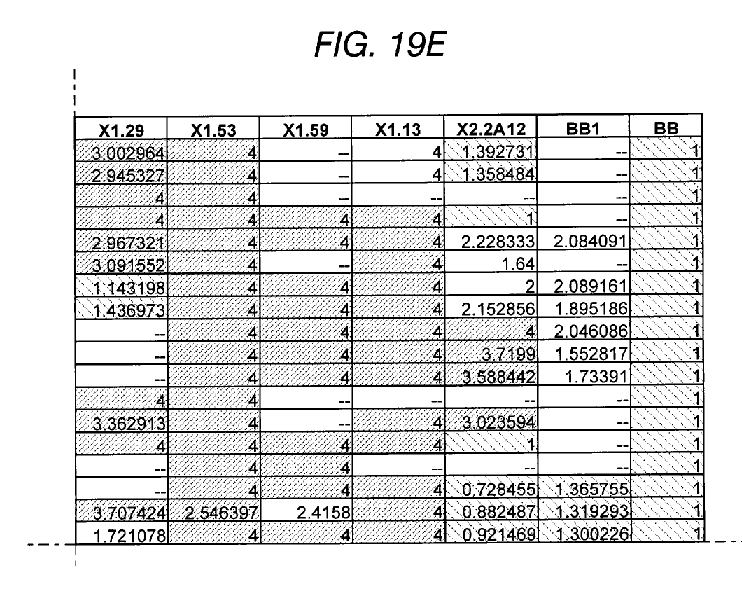

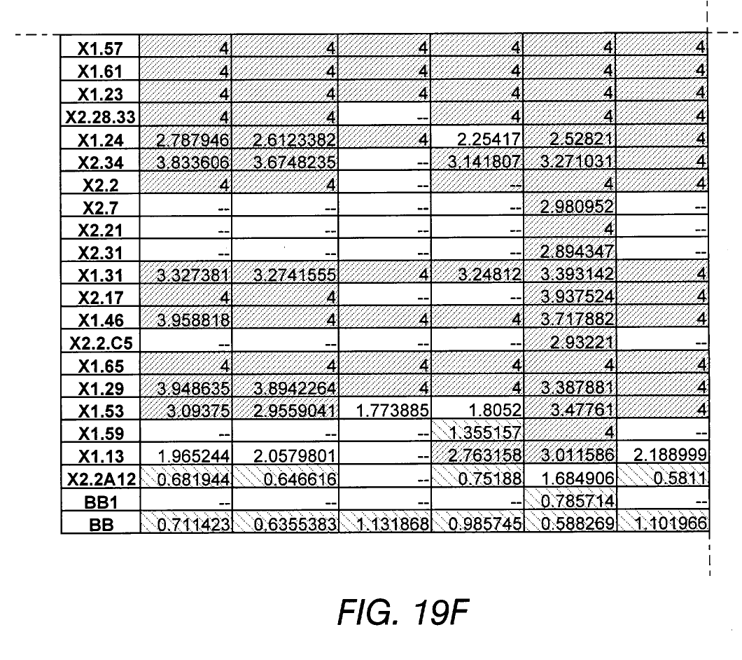

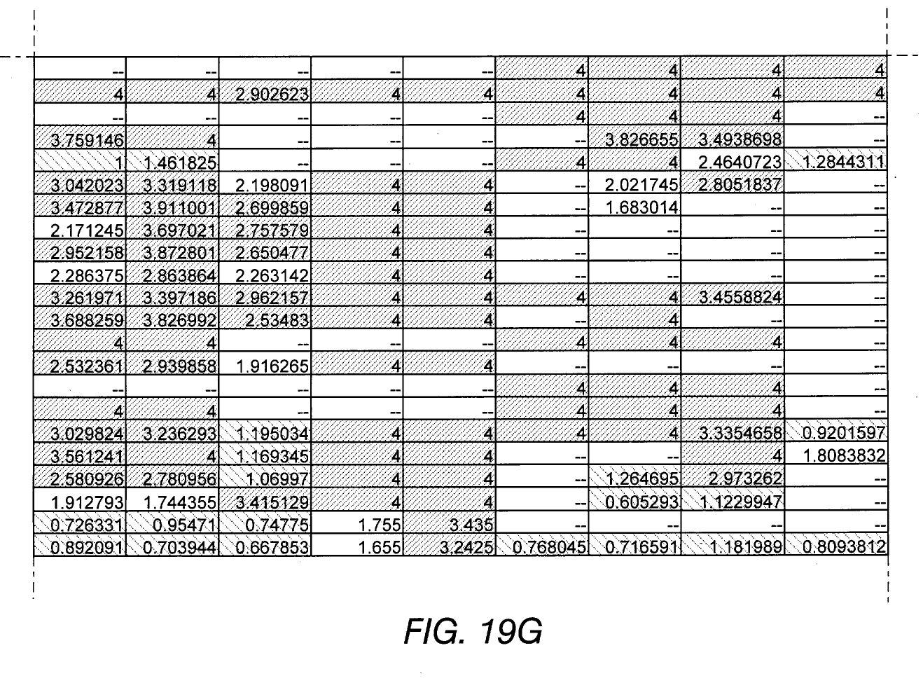

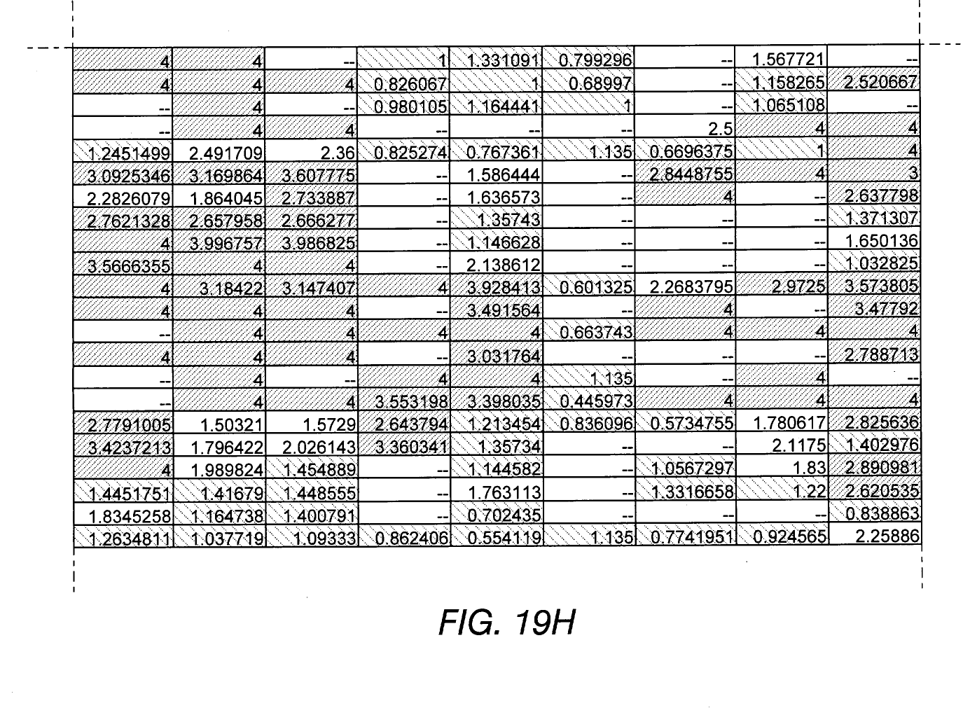

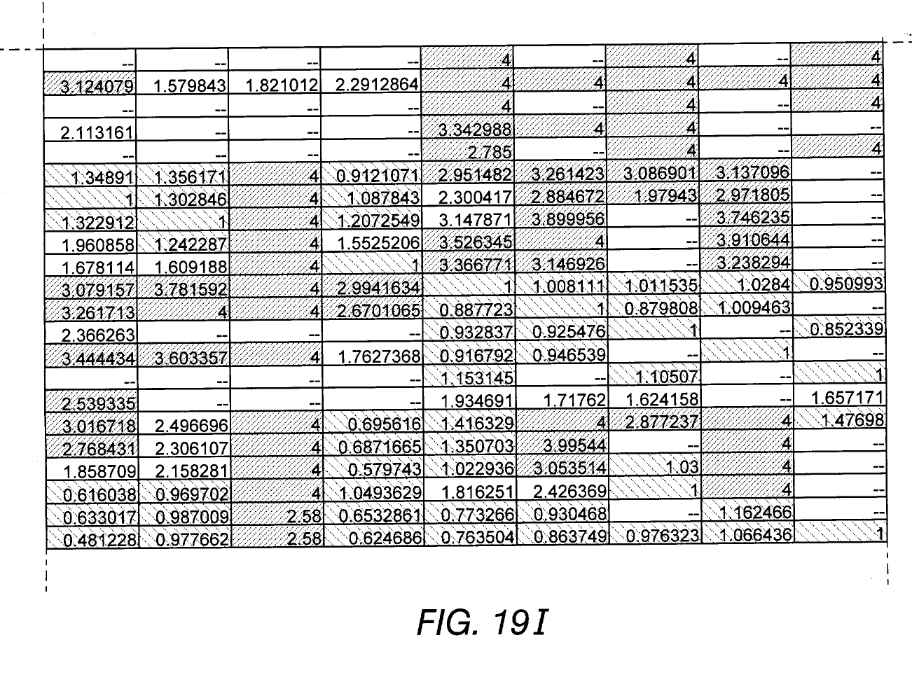

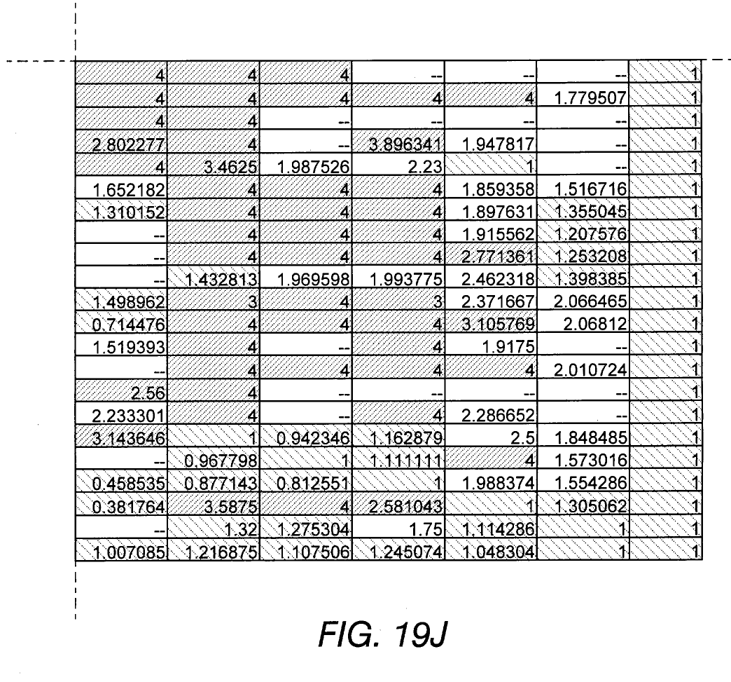

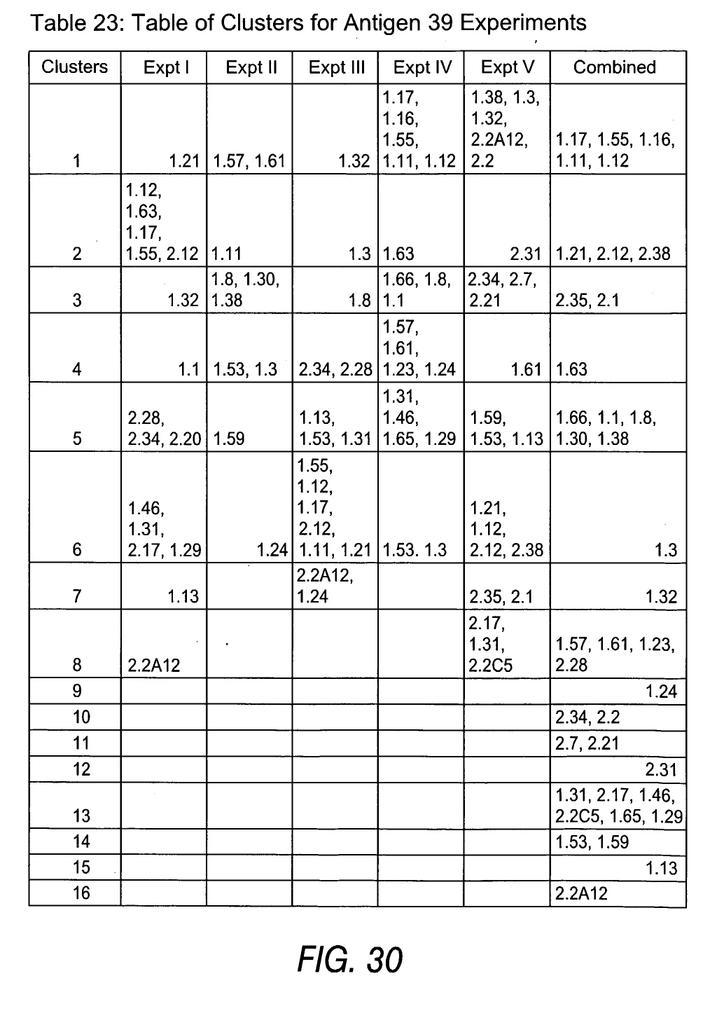

For five experiments using ANTIGEN39 antibodies, the clustering results for the five input data sets showed that there were a large number of clusters of varying degree of similarity, suggesting the presence of several different epitopes, some of which may overlap. This is shown in FIG. 6A, FIG. 18, FIG. 19, and FIG. 30. For example, the cluster containing antibodies 1.17, 1.55, 1.16, 1.11, and 1.12 and the cluster containing 1.21, 2.12, 2.38, 2.35, and 2.1 are fairly closely related, as each antibody pair shows no more than 25% difference, with the exception of 2.35 and 1.11. This high degree of similarity across the two clusters suggested that the two different epitopes may have a high degree of similarity

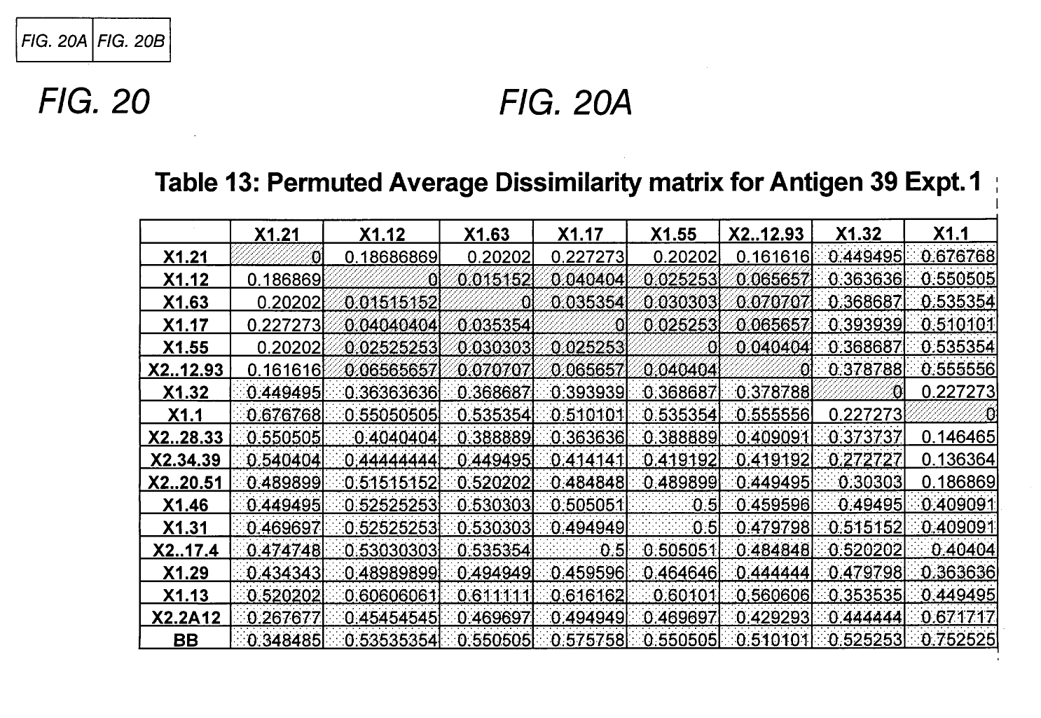

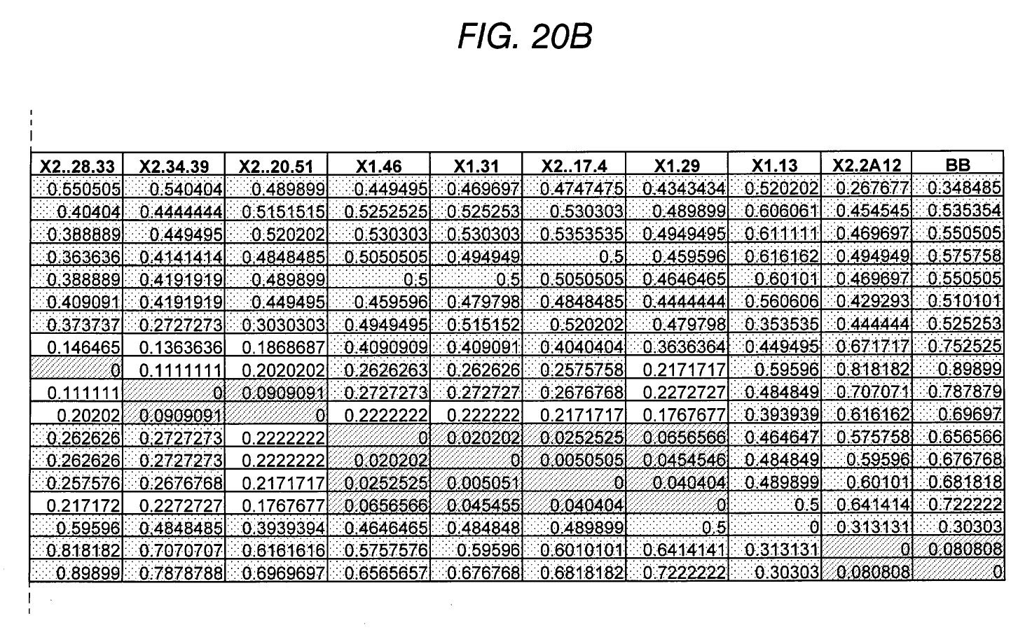

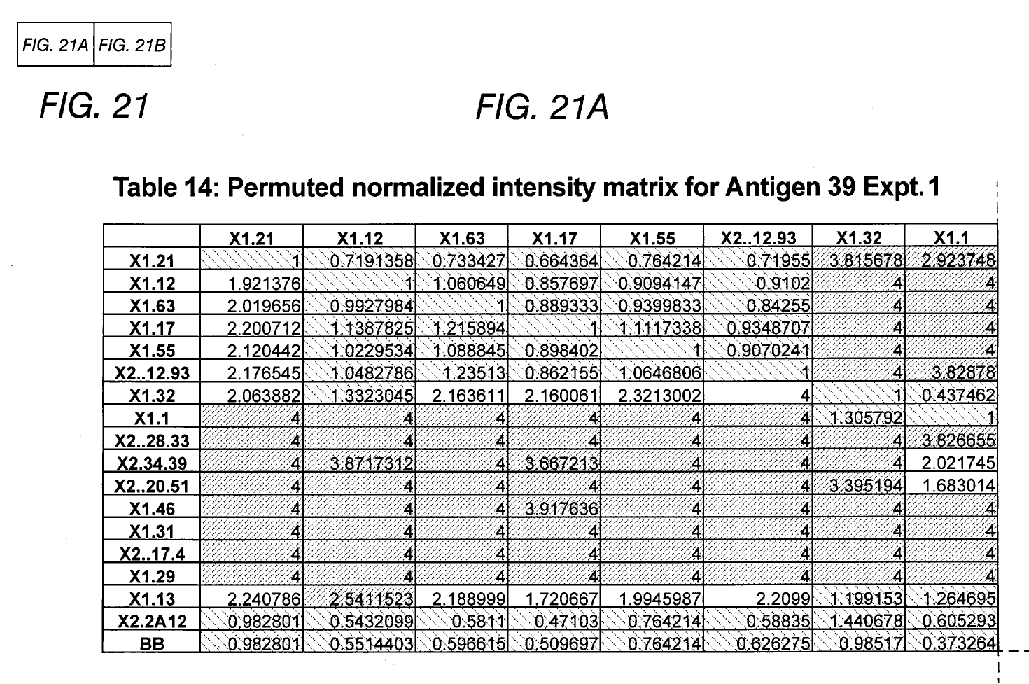

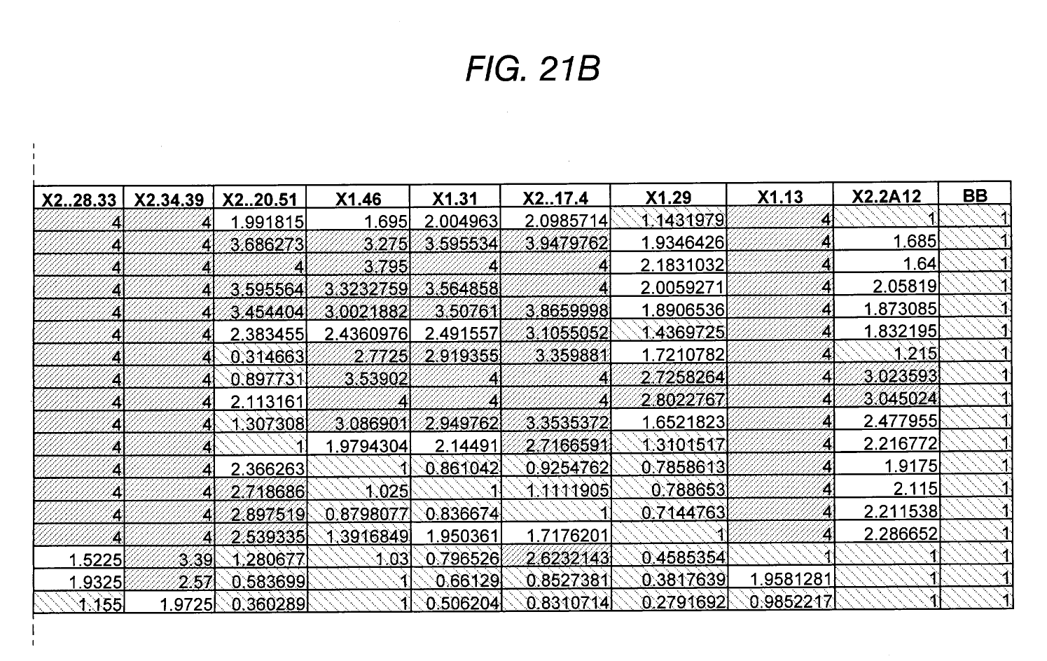

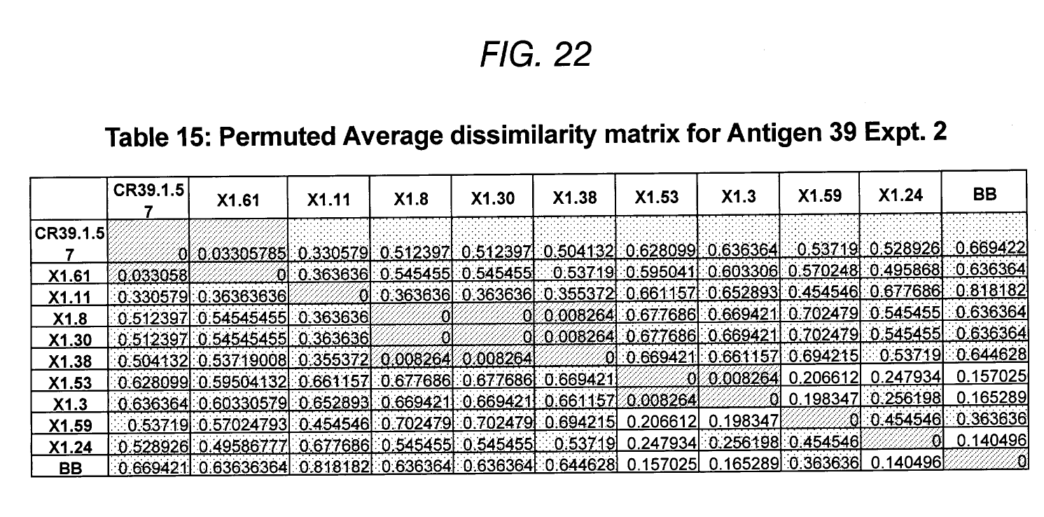

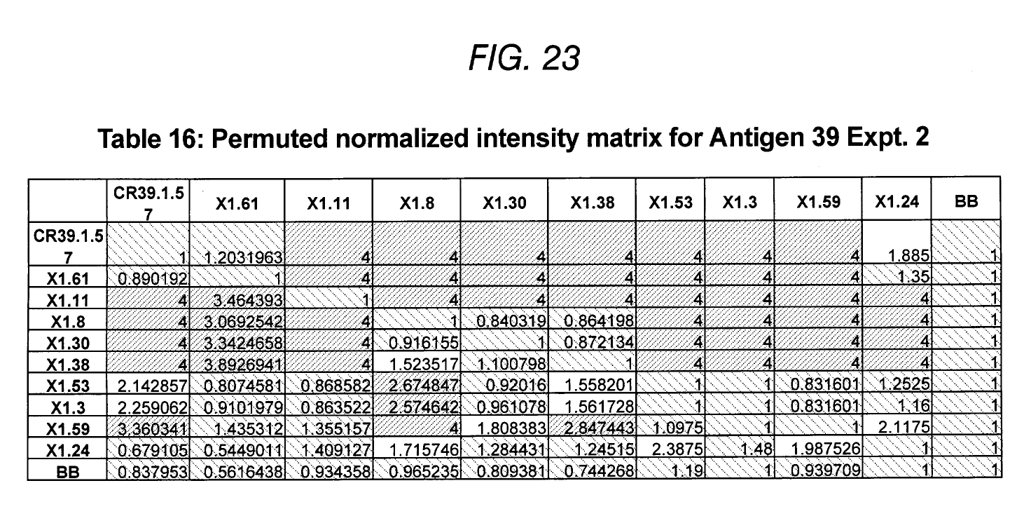

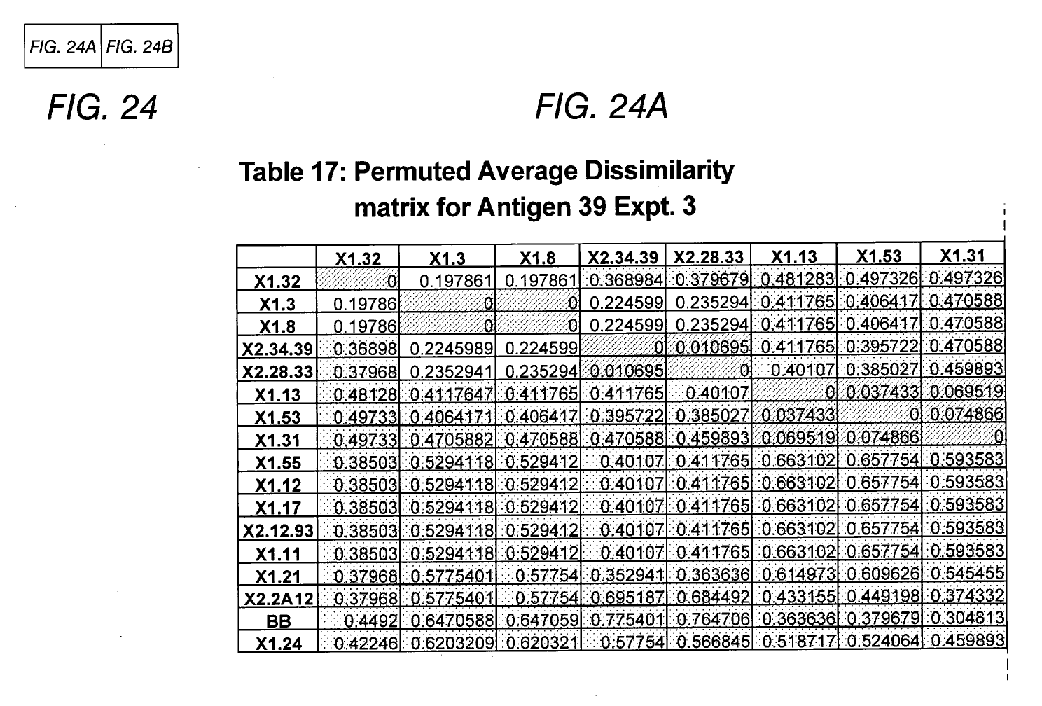

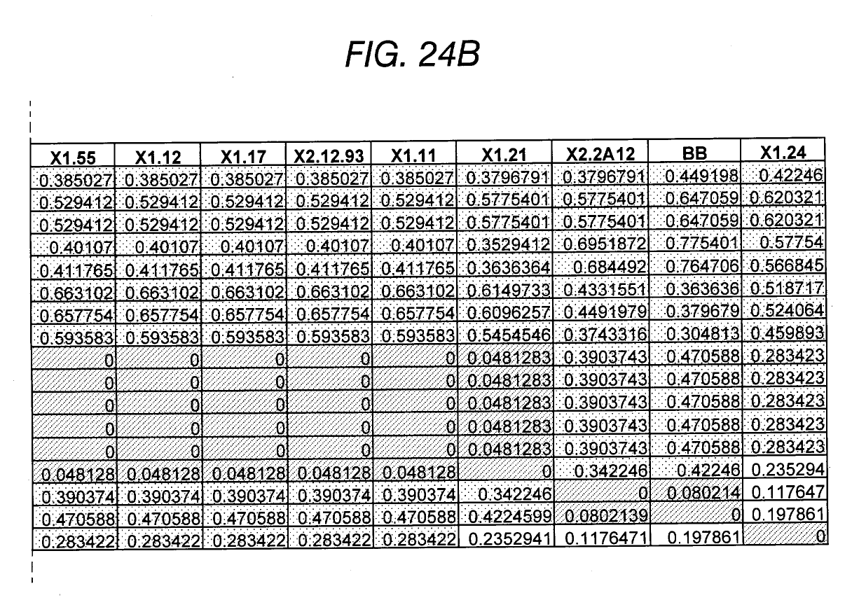

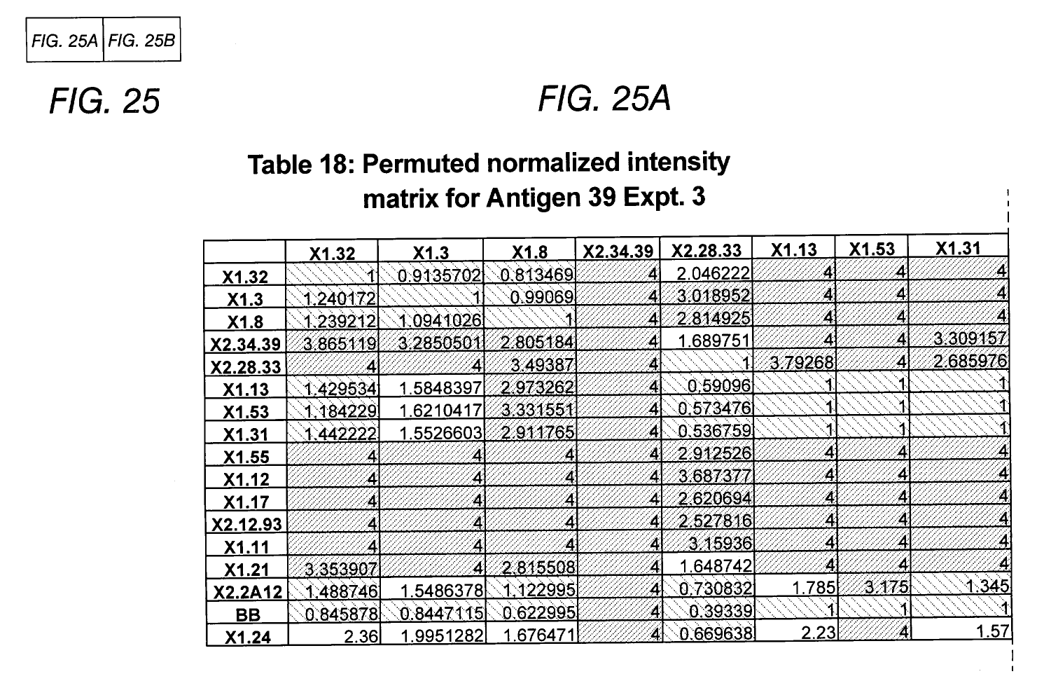

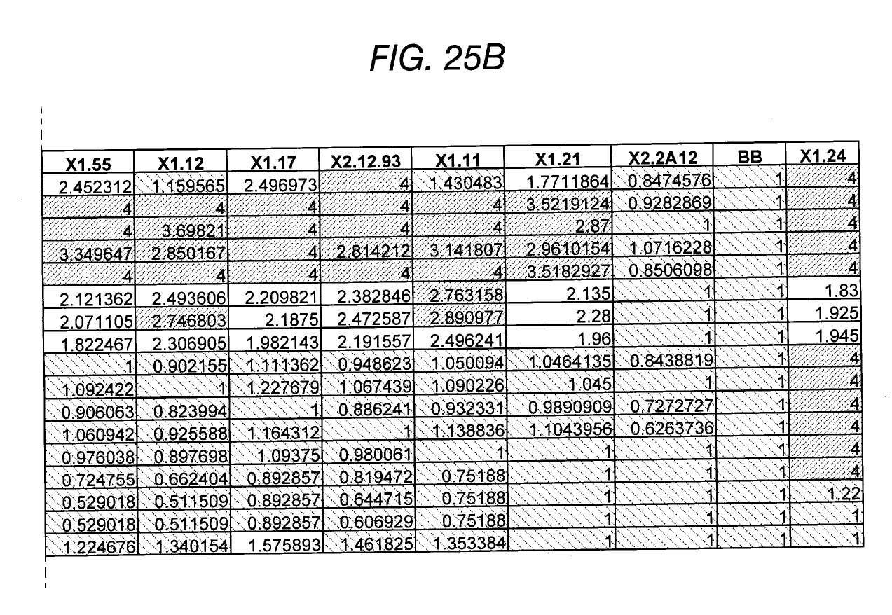

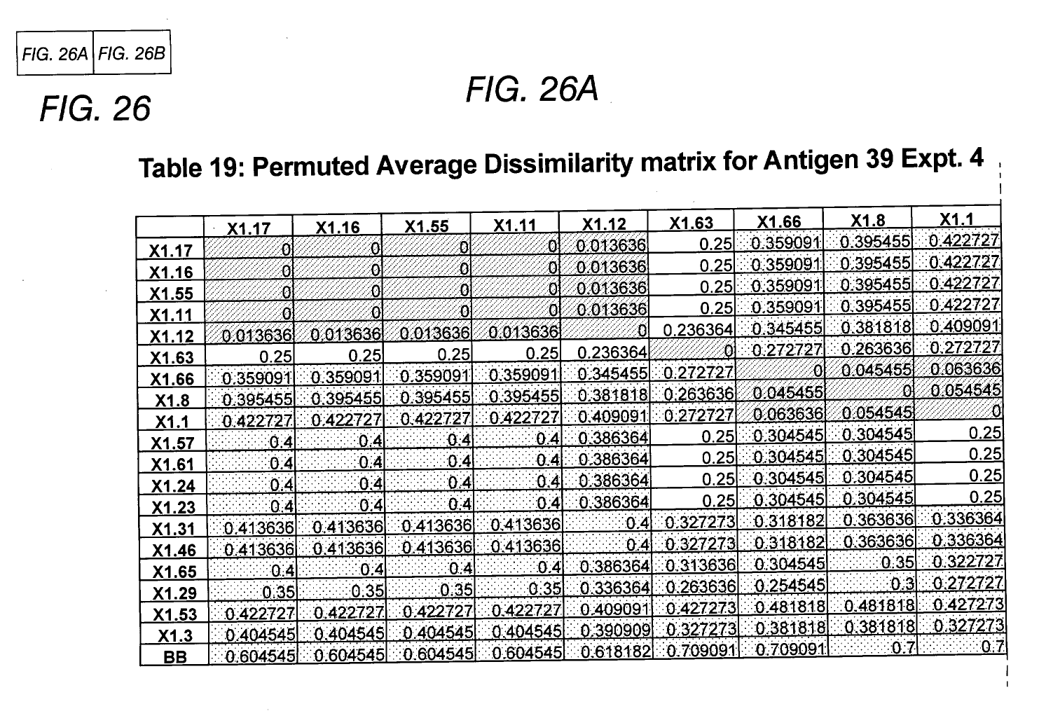

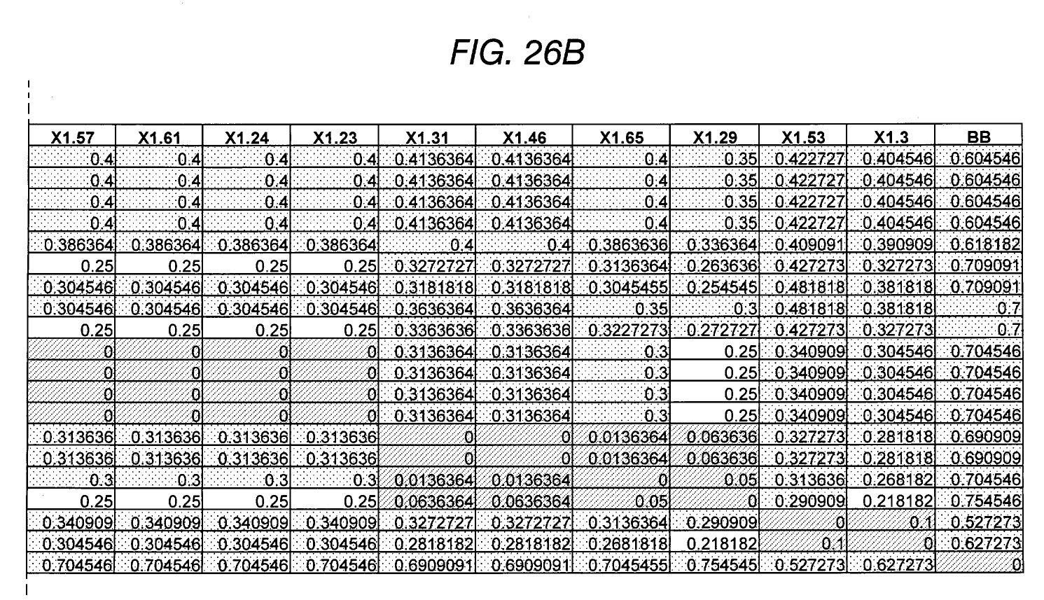

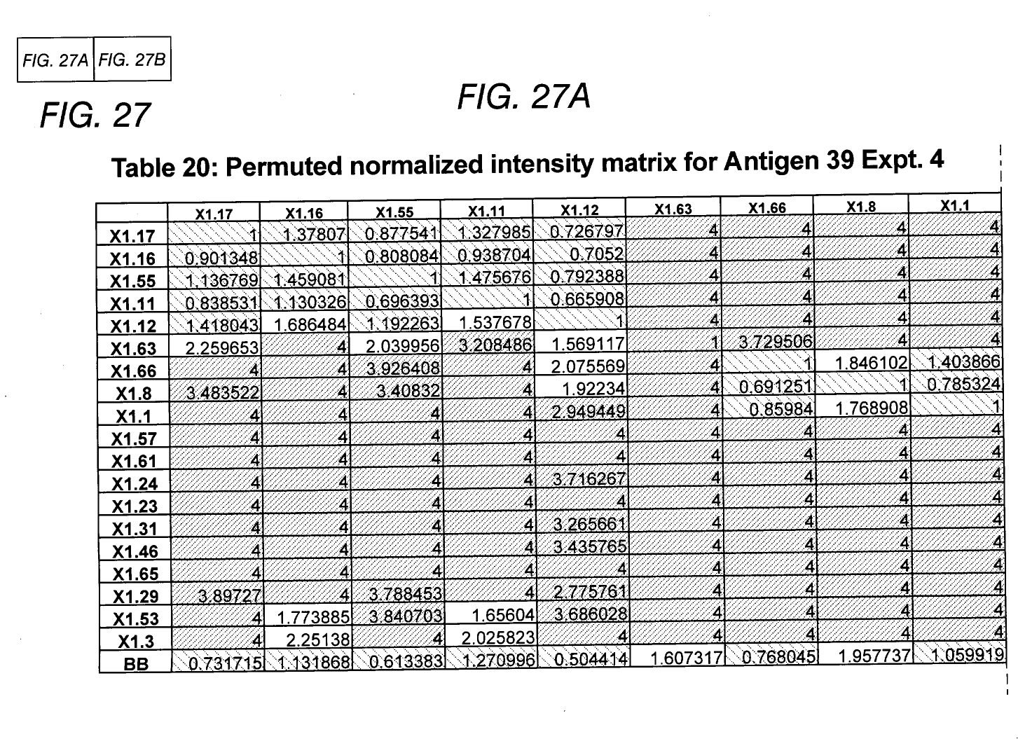

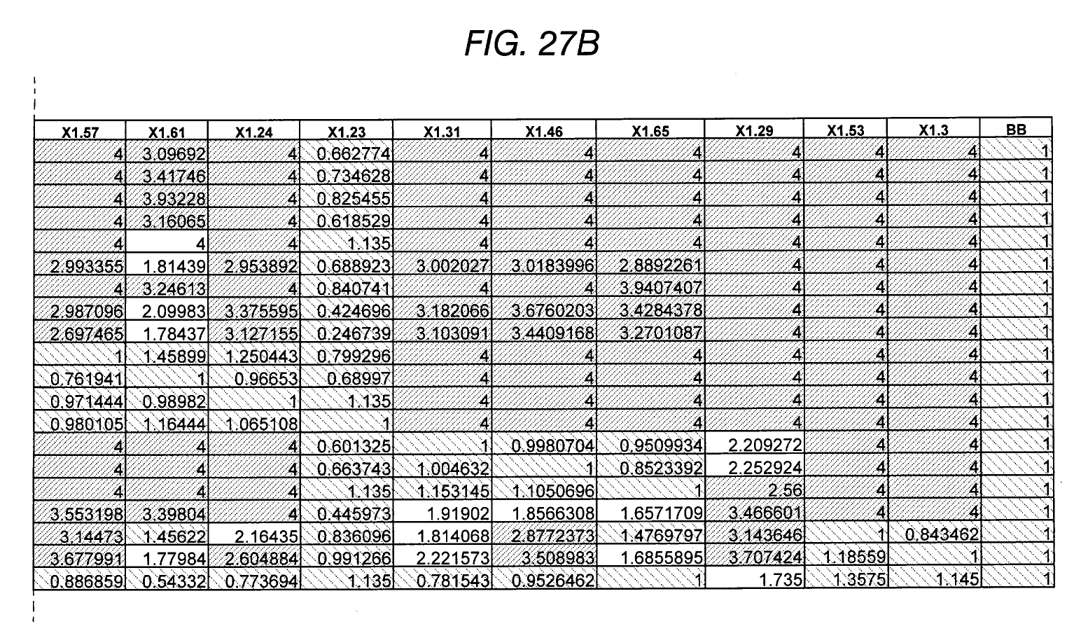

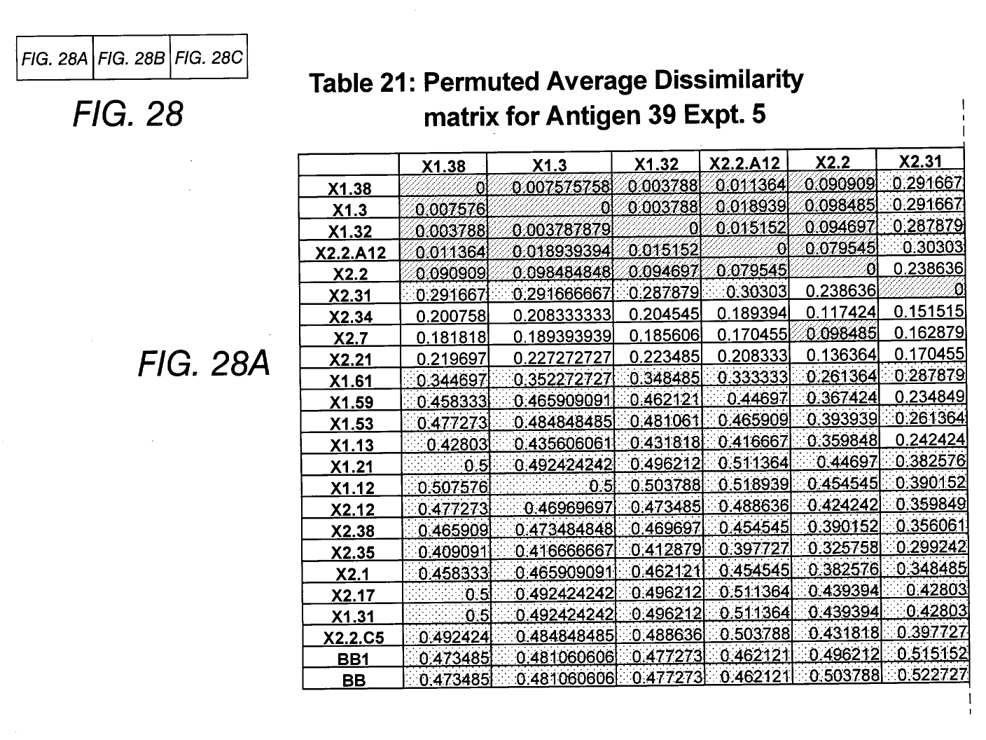

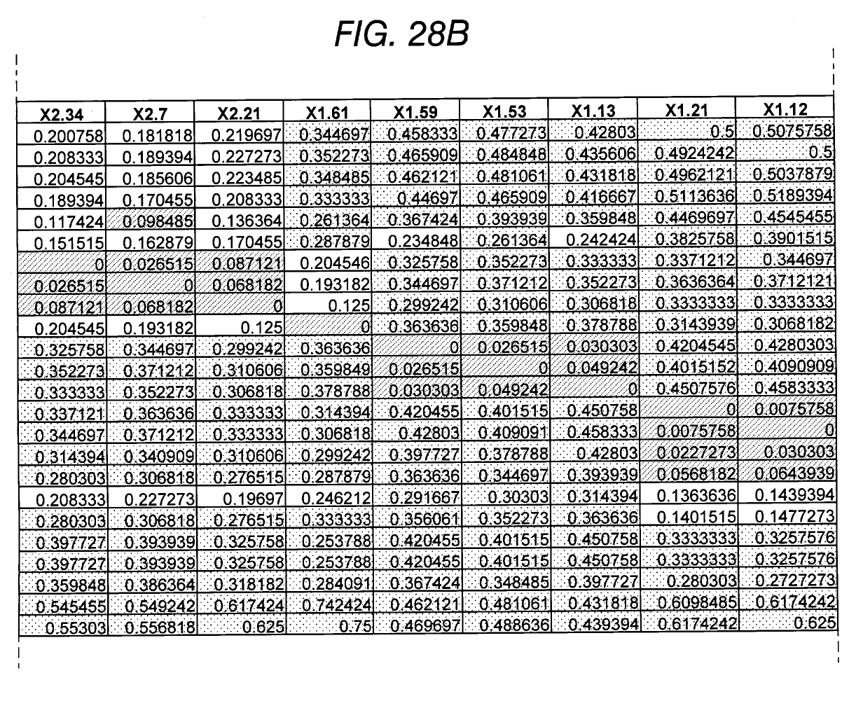

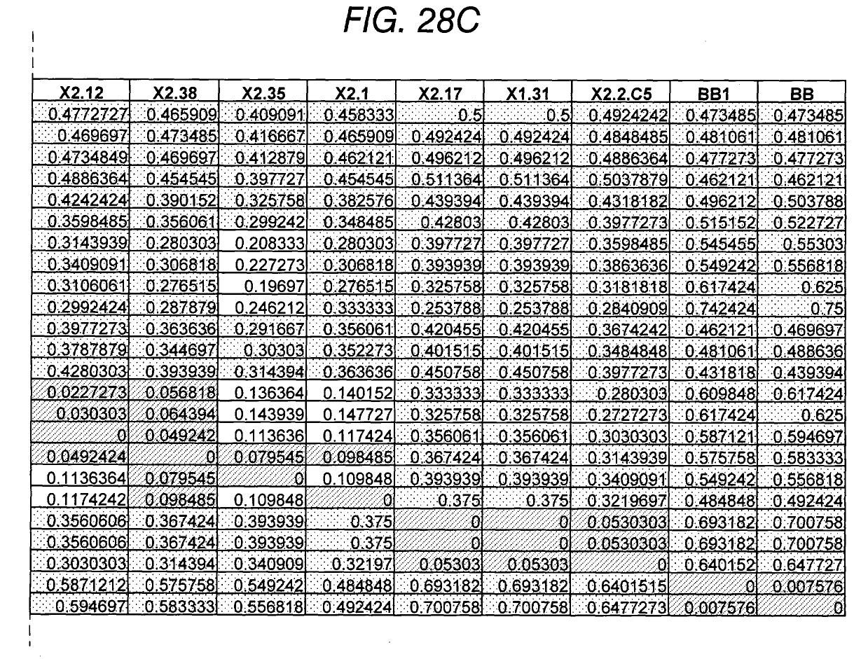

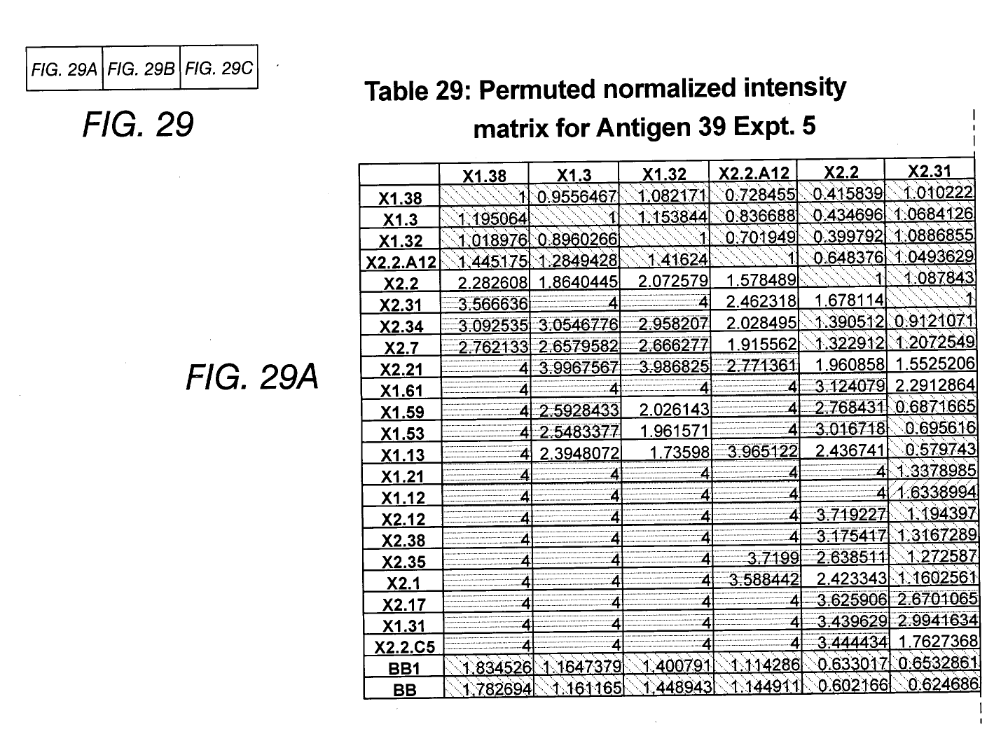

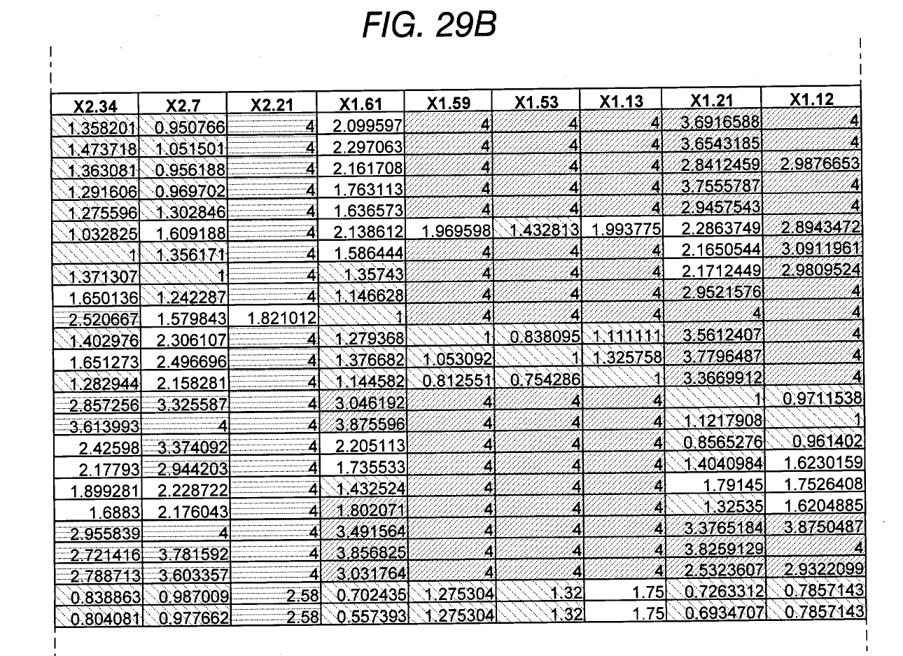

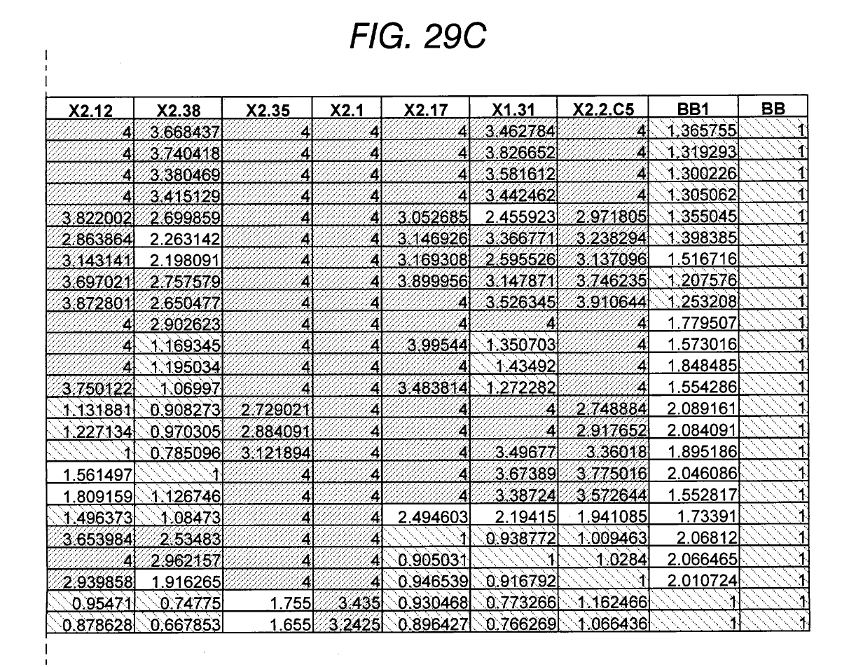

The five data sets from separate experiments using ANTIGEN39 antibodies were also independently clustered, to demonstrate that the processes disclosed and claimed herein produce consistent clustering results. Clustering results are summarized in FIGS. 6B-6F and in FIGS. 20-30, where FIG. 30 summarizes the clusters for each of the individual data sets and for the combined data set with all of the antibodies for the five experiments. FIG. 6B shows the dendrogram for the ANTIGEN39 antibodies for Experiment 1: Antibodies 1.12, 1.63, 1.17, 1.55, and 2.12 consistently clustered together in this experiment as well as in other experiments as do antibodies 1.46, 1.31, 2.17, and 1.29. FIG. 6C shows the dendrogram for the ANTIGEN39 antibodies for Experiment 2: Antibodies 1.57 and 1.61 consistently clustered together in this experiment as well as in other experiments.

FIG. 6D shows the dendrogram for the ANTIGEN39 antibodies for Experiment 3: Antibodies 1.55, 1.12, 1.17, 2.12, 1.11, and 1.21 consistently clustered together in this experiment as well as in other experiments. FIG. 6E shows the dendrogram for the ANTIGEN39 antibodies for experiment 4: Antibodies 1.17, 1.16, 1.55, 1.11, and 1.12 consistently clustered together in this experiment as well as in other experiments as do antibodies 1.31, 1.46, 1.65, and 1.29, as well as antibodies 1.57 and 1.61. FIG. 6F shows the dendrogram for the ANTIGEN39 antibodies for experiment 5: Antibodies 1.21, 1.12, 2.12, 2.38, 2.35, and 2.1 consistently clustered together in this experiment as well as in other experiments.

In general, the clustering algorithm produced consistent results both among the individual experiments and between the combined and individual data sets. Antibodies which cluster together or are in neighboring clusters for multiple individual data sets also cluster together or be in neighboring clusters for the combined data set. For example, cells having lighter shading indicate antibodies that consistently clustered together in the combined data set and in all of the data sets in which they were present (Experiments 1, 3, 4, and 5). These results indicate that the algorithm produces consistent clustering results both across multiple individual experiments and that it retains the consistency upon the merging of multiple data sets.

Finally, there is a high level of self-consistency for the data with regard to revealing whether or not two antibodies compete for the same epitope. The percent of antibody pairs for which the data consistently reveals whether or not they compete for the same epitope is summarized for each data set in Table 2, below, which reveals that the consistency was nearly 90% for four out of the five individual data sets as well as for the combined data set.

TABLE 2
Percent Consistency Values for ANTIGEN39
Antibody Experiments

| Experiment | % Consistency |
|---|---|
| 1 | 92 |
| 2 | 82 |
| 3 | 88 |
| 4 | 92 |
| 5 | 88 |
| Combined | 88 |

### Consistency of Epitope Binning Results With Flow Cytometry (FACS) Results

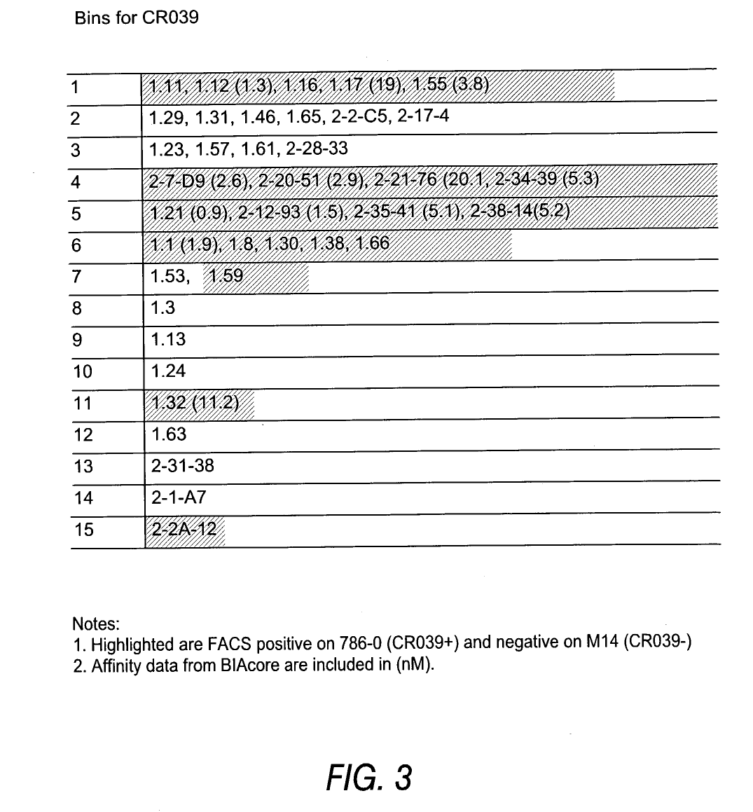

Results from the embodiment described in Example 3 below, using antibodies against the ANTIGEN39 target further demonstrate that results generated by epitope binning according to the methods of the present invention are consistent with the results generated using flow cytometry (fluorescence-activated cell sorter, FACS). Cells expressing ANTIGEN39 were sorted by FACS, and ANTIGEN39-negative cells were used as negative controls also sorted by FACS. The cell surface binding sites recognized by antibodies from different bins represent different epitopes. FIG. 3 shows a comparison of results from antibody experiments using the anti-ANTIGEN39 antibody, with results using FACS. As shown in FIG. 3, the antibodies in a given bin are either all positive (Bins 1,4,5) or all negative (bins 2 and 3) in FACS, which indicates that the antibody epitope binning assay indeed bins antibodies based on their epitope binding properties. Thus, epitope binning, as described herein, provides an efficient, rapid, and reliable method for determining the epitope recognition properties of antibodies, and sorting and categorizing antibodies based on the epitope they recognize.

### Alternative Data Analysis Process and Consistency of Epitope Binning With Sequence Results

An alternative data analysis process involves subtracting the data matrix for the experiment carried out with antigen from the data matrix for the experiment without antigen to generate a normalized background intensity matrix. The value in each diagonal cell is then used as a background value for determining the binding affinity of the antibody in the corresponding column. Cells in each column the normalized background intensity matrix (the subtracted matrix) having values significantly higher than the value of the diagonal cell for that column are highlighted or otherwise noted. Generally, a value of about two times the corresponding diagonal is considered “significantly higher”, although one of skill in the art can determine what increase over background is the threshold for “significantly higher” in a particular embodiment, taking into account the reagents and conditions used, and the “noisiness” of the input data. Columns with similar binding patterns are grouped as a bin, and minor differences within the bin are identified as sub-bins. This data analysis can be carried out automatically for a given set of input data. For example, input data can be stored in a computer database application where the cells in diagonal are automatically marked, and the cells in each column as compared with the numbers in diagonal are highlighted, and columns with similar binding patterns are grouped.

In a preferred embodiment using fifty-two (52) antibodies against ANTIGEN54, binning results using the data analysis process described above correlated with sequence analysis the CDR regions of antibodies binned using the MCAB competitive antibody assay. The 52 antibodies consisted of 2 or 3 clones from 20 cell lines. As expected, sequences of clones from same line were identical, so only one representative clone from each line was sequenced. The correspondence between the epitope binning results and sequence analysis of antibodies binned by this method indicates this approach is suitable for identifying antibodies having similar binding patterns. In addition, correspondence between the epitope binning results and sequence analysis of antibodies binned by this method means that the epitope binning method provides information and guidance about which antibody sequences are important in determining the epitope specificity of antibody binding.

### Limiting Dilution Assays

During a standard assay using moderate to high concentrations of target, a collection of different antibodies having different affinities for the same target antigen may generate signals of equal or similar intensity. However, as the amount of antigen is diluted, it becomes possible to discern differences in affinity among the antibodies. Using limiting concentrations of target antigen in the assay in accordance with the teachings of the present disclosure, it is possible to establish a kinetic ranking of a collection of antibodies against the same target antigen.

Under conditions of limiting amounts of antigen, a collection of antibodies against the same antigen will give a range of signals from high to low or no signal, even though in the original assay, using high to moderate levels of antigen, some of these antibodies may have produced signals of similar apparent strength. Antibodies can thus be affinity-ranked by their signal intensity in a limiting antigen assay carried out in accordance with the teachings of the present disclosure.

Embodiments of the invention relate to methods for rapidly determining the differential binding properties within a set of antibodies. Accordingly, rapid identification of optimal antibodies for binding to a target can be determined. Any set of antibodies raised against a particular target antigen may bind to a variety of epitopes on the antigen. In addition, antibodies might bind to one particular epitope with varying affinities. Embodiments of the invention provide methods for determining how strongly or weakly an antibody binds to a particular epitope in relation to other antibodies generated against the antigen.

One embodiment of the invention is provided by preparing a set of diluted antigen preparations and thereafter measuring the binding of each antibody in a set of antibodies to the diluted antigen preparations. A comparison of each antibody's relative affinity for a particular concentration of antigen can thereby be performed. Accordingly, this method discerns which antibodies bind to the more dilute concentration of antigen, or to the more concentrated antigen preparations, as part of a comparative assay for the relative affinity of each antibody in a set.

Another embodiment of the invention is provided by preparing a set of diluted antibody preparations and thereafter measuring the binding of an antigen to each of the diluted antibody preparations. A comparison of each antibody's relative affinity for a particular antigen can thereby be performed. Accordingly, this method discerns whether a particular concentration of an antigen binds to the more dilute concentration of antibody preparations, or to the more concentrated antibody preparations, as part of a comparative assay for the relative affinity of each antibody in a set.

Although a process is disclosed in which an antibody's relative affinity can be determined, a similar protocol can be foreseen for the identification of high affinity antibody fragments, protein ligands, small molecules or any other molecule with affinity toward another. Thus, the invention is not limited to only analyzing binding of antibodies to antigens.

One embodiment of the invention provides a method for analyzing the kinetic properties of antibodies to allow ranking and selection of antibodies with desired kinetic properties. Affinity, as defined herein, reflects the relationship between the rate at which one molecule binds to another molecule (association constant, Kₒₙ) and the rate at which dissociation of the complex occurs (dissociation constant, K_{off}). When an antibody and target are combined under suitable conditions, the antibody will associate with the target antigen. At some point the ratio of the amount of antibody binding and releasing from its target reaches an equilibrium. This equilibrium is referred to as the “affinity constant” or just “affinity”.

When binding reactions having identical concentrations of antibody and target molecule are compared, reactions containing higher affinity antibodies will have more antibodies bound to the target at equilibrium than reactions containing antibodies of lower affinity.

In assays where the binding of one molecule to another is measured by the formation of complexes which generate a signal, the amount of signal is proportional to the concentrations of the molecules as well as to the affinity of the interaction. For purposes of the present disclosure, assays are employed to measure formation of complexes between antibodies and their targets (on antigens), where signals being measured in such assays may be proportional to the concentrations of antibody or antibodies, concentration of target antigen, and the affinity of the interaction. Suitable assay methods for measuring formation of antibody-target complexes include enzyme linked immunosorbent assays (ELISA), fluorescence-linked immunosorbent assays (including Luminex systems, FMAT and FACS sytems), radioisotopic assay (RIA) as well as others which can be chosen by one of skill in the art.

Another aspect of the present invention includes methods for kinetically ranking antibodies by affinity based on the signal strength of an assay such as an assay listed above, when the target or antigen is provided at limiting concentrations. Antibody and antigen are combined, the binding reaction is allowed to go to equilibrium, and after equilibrium is achieved, an assay is performed to determine the amount of antibody bound to the target or antigen. According to one aspect of the present invention, the amount of bound antibody detected by the assay is directly proportional to the affinity of the antibody for the target or antigen. At very low concentrations of antigen, some antibodies of low affinity will not generate a detectable signal due to an insufficient amount of bound antibody. At the same very concentrations of antigen, antibodies of moderate affinity will generate low signals, and antibodies with high affinity will generate strong signals.

During a standard assay using moderate to high concentrations of target, a collection of different antibodies having different affinities for the same target antigen may generate signals of equal or similar intensity. However, as the amount of antigen is diluted, it becomes possible to discern differences in affinity among the antibodies. Using limiting concentrations of target antigen in the assay in accordance with the teachings of the present disclosure, it is possible to establish a kinetic ranking of a collection of antibodies against the same target antigen.

Under conditions of limiting amounts of antigen, a collection of antibodies against the same antigen will give a range of signals from high to low or no signal, even though in the original assay using high to moderate levels of antigen, some of these antibodies may have produced signals of similar apparent strength. Antibodies can thus be affinity-ranked by their signal intensity in a limiting antigen assay carried out in accordance with the teachings of the present disclosure.

Another aspect of the invention is a method of determining antibodies with higher affinities than currently known and characterized antibodies. This method involves using the characterized antibodies as kinetic standards. A plurality of test antibodies are then measured against the kinetic standard antibodies to determine those antibodies that bind to more dilute antigen preparations than to the standard antibodies. A plurality of test antibodies is then measured against the kinetic standard antibody to determine those antibodies which have more antibody bound to a given dilute preparation of antigen. This allows the rapid discovery of antibodies that have a higher affinity for antigen in comparison to the kinetic standard antibodies.

In one preferred embodiment, an ELISA is used in a limiting antigen assay in accordance with the present disclosure.

It has been empirically determined that supernatants of cultured B-cells generally secrete antibodies in a concentration range from 20 ng/ml to 800 ng/ml. Because there is often a limited amount of supernatant from these cultures, B-cell culture supernatants are typically diluted 10-fold for most assays, giving a working concentration of from 2 ng/ml-80 ng/ml for use in affinity determination assays. In one aspect of the invention, the appropriate concentration of target antigen used to coat ELISA plates was determined by using a reference solution from a monoclonal antibody at a concentration of 100 ng/ml. This number could change depending on the concentration range of test antibodies and the affinity of the reference antibody, such that the concentration of target antigen required to give half-maximal signal in a ELISA-based measurement of antibody/antigen binding can be empirically determined. This determination is discussed in more detail below.

Antigen at an empirically determined optimal coating concentration was used in affinity measurement assays to discern the antibodies produced by various B-cell cultures that gave an ELISA value higher than a reference monoclonal antibody. According to the methods of the present invention, the only way to obtain a higher signal than that obtained using the reference antibody is if (1) the antibody is of higher affinity than the reference antibody or (2) the antibody has the same affinity but is present in a higher concentration that the reference monoclonal antibody. As disclosed previously, antibodies in B-cell culture supernatants are usually at concentrations of between 20-800 ng/ml and are diluted to a working concentration of between 2 to 80 ng/ml. In one embodiment, test antibodies at a concentration of between 2 to 80 ng/ml are used in assays having a reference antibody concentration of 100 ng/ml. The signal achieved from the test antibodies is compared to that of the 100 ng/ml reference antibody. If antibodies within the test group are found to have a higher signal, then the antibody is assumed to be of a higher affinity than the reference antibody.

In another embodiment, antibodies generated from hybridomas were ranked using a limiting kinetic antigen assay in an ELISA-based protocol. The binding affinities for these antibodies was confirmed by quantifying and kinetically ranked the antibodies using a Biacore system. As is known, the Biacore system gives formal kinetic values for the binding coefficient between each antibody and the antigen. It was determined that the kinetic ranking of antibodies using the limiting antigen assay as taught by the present disclosure closely correlated with the formal kinetic values for these antibodies as determined by the Biacore method, as shown below.

Briefly, the Biacore technology uses surface plasmon resonance (SPR) to measure the decay of antibody from antigen at various concentrations of antigen and at a known concentration of antibody. For example, chips are loaded with antibody, washed, and the chip is exposed to a solution of antigen to load the antibodies with antigen. The chip is then continually washed with a solution without antigen. An initial increase in SPR is seen as the antibody and antigen complex forms, followed by decay as the antigen-antibody complex dissociates. This decay in signal is directly proportional to antibody affinity. Similarly this method could run the reverse assay with limited concentrations of antibody coated on the chip.

Using the Luminex (MiraiBio, Inc., Alameda, Calif.) technology antibodies are assayed for how they bound a plurality of different antigen coated beads. In this assay each bead set is preferably coated with a different concentration of antigen. As the Luminex reader has the ability to multiplex all the beads sets, the bead sets are combined and antibody binding to each of the different bead sets are determined. The behavior of antibodies on the differentially coated beads can then be tracked. Once normalized for antibody concentration, then antibodies which maintain a high degree of binding as one moves from non-antigen limiting concentrations to limited antigen concentrations correlate well to high affinity. Advantageously, these differential shifts can be used to relatively rank antibody affinities. For example, samples with smaller shifts correspond to higher affinity antibodies and antibodies with larger shifts correspond to lower affinity antibodies.

TABLE 3
Comparison of Affinity Rankings Between
Biacore and Luminex Methods

| BiaCore Affinity Measurements | | | | |
|---|---|---|---|---|
| | | Biacore | | |
| ka (M − 1 | | Med-res | | Luminex |
| s − 1) | kd (s − 1) | KD (nM) | Rank | rank |
| 9.9 × 10.5 | 9.3 × 10 − 3 | 9.4 | 1 | 1 |
| 2.7 × 10.5 | 4.2 × 10 − 3 | 16 | 2 | 14 |
| 3.1 × 10.5 | 5.6 × 10 − 3 | 18 | 3 | 57 |
| 8.2 × 10.5 | 2.7 × 10 − 2 | 33 | 4 | 83 |
| 1.4 × 10.6 | 6.2 × 10 − 2 | 42 | 5 | 116 |
| 2.9 × 10.5 | 1.6 × 10 − 2 | 54 | 6 | 123 |

In another embodiment of the invention, a series of limited concentrations of the antibody being tested are compared to a standard solution of antibody. Such a method using limiting concentrations of antibody would appear to be a “reverse” of the method using limiting antigen concentrations, but it provides a similar mechanism for rapidly screening a set of antibodies to determine each antibody's relative affinity for the target antigen. Other plates that are, or can be, chemically modified to allow covalent or passive coating can also be used. One of skill in the relevant art can devise further modifications of the methods presented herein to carry out an assay using limiting antibody dilution to screen and kinetically rank test antibodies.

### Determining Optimal Bound Antigen Concentration

Embodiments of the limiting antigen assay method are practiced using a method by which antigen is bound or attached to a stationary surface prior to subsequent manipulations. The surface is preferably part of a vessel in which subsequent manipulations may occur; more preferably, the surface is in a flask or test tube, even more preferably the surface is in the well of a microtiter plate such as a 96-well plate, a 384-well plate, or a 864-well plate. Alternately, the surface to which antigen is bound may be part of a surface such as a slide or bead, where the surface with bound antigen may be manipulated in subsequent antibody binding and detection steps. Preferably, the process by which the antigen is bound or attached to the surface does not interfere with the ability of antibodies to recognize and bind to the target antigen.

In one embodiment, the surface is coated with streptavidin and the antigen is biotinylated. In a particularly preferred embodiment, the plate is a microtiter plate, preferably a 96-well plate, having streptavidin coating at least one surface in each well, and the antigen is biotinylated. Most preferably, the plate is Sigma SA 96-well plate and the antigen is biotinylated with Pierce EZ-link Sulpho-NHS Biotin (Sigma-Aldrich Canada, Oakville Ontario, CANADA). Alternative methods of biotinylation which attach the biotin molecule to other moieties can also be used.

In the unlikely event that an antigen cannot be biotinylated, alternative surfaces to which antigen can be bound can be substituted. For example, the Costar® Universal-BIND™ surface, which is intended to covalently immobilize biomolecules via an abstractable hydrogen using UV illumination resulting in a carbon-carbon bond. (Corning Life Sciences, Corning, N.Y.). Plates, for example, Costar® Universal-BIND™ 96-well plates, may be used. One of skill in the art can modify subsequent manipulations in the event that the use of alternate surfaces such as Costar® Universal-BIND™ increases the time of the assay and/or requires the use of more antigen.

In one embodiment of the present invention, a “checkerboard” assay design is used to find optimal concentration of bound antigen. One example is shown below in Table 5. The following description includes a disclosure of the steps to determine the optimal coating concentration of biotinylated antigen using 96-well plates coated with streptavidin. This disclosure is intended merely to illustrate one way to practice various aspects of the present invention. The scope of the present invention is not limited to the methods of the assay described above and below, as one of skill in the art can practice the methods of the present invention using a wide variety of materials and manipulations. Methods including but not limited to; expression of antigen on cells (transient or stable), using phage which express different copy number of antigen per phage.

### Antigen Dilution and Distribution

An antigen to be tested is selected. Such an antigen may be, for example, any antigen that might provide a therapeutic target by antibodies. For example, tumor markers, cell surface molecules, Lymphokines, chemokines, pathogen associated proteins, and immunomodulators are non-limiting examples of such antigens.

A solution of antigen at an initial concentration, preferably about 1 ug/ml, is diluted in a series of stepwise dilutions. Diluted samples are then placed on surfaces such as in the wells of a microtiter plate, and replicates of each sample are also distributed on surfaces. Antigen solutions may contain blocking agents if desired. In a preferred embodiment, serial dilutions of antigen are distributed across the columns of a 96-well plate. Specifically, a different antigen dilution is placed in each column, with replicate samples in each row of the column. In a 96-well plate, replicates of each dilution are placed in rows A-H under each column. Although the standard dilutions vary from antigen to antigen, the typical dilution series starts at 1 μg/ml and is serially diluted 1:2 to a final concentration of about 900 pg/ml.

In one embodiment, biotinylated antigen is diluted from a concentration of 1 ug/ml to 900 pg/ml horizontally across a 96 well plate. While a preferred blocking buffer is a PBS/Milk solution, others buffers such as BSA diluted in PBS can be substituted. In another embodiment, biotinylated antigen is diluted from a concentration of 1 ug/ml to 900 pg/ml in 1% skim milk/1×PBS pH 7.4, and pipetted into the wells of columns 1 to 11 of a Sigma SA (streptavidin) microtiter plate, with 8 replicates of each dilution placed in rows A-H of each column. Column 12 is left blank, serving as the “antibody-only” control. The final volume in each well is 50 ul. Antigen is incubated on the surface (e.g., in the wells of the plate) for a suitable amount of time for the antigen to become attached to the surface; incubation time, temperature, and other conditions can be determined from manufacturer's instructions and/or standard protocols for the surface being used. After incubation, excess antigen solution is removed. If needed, plates are then blocked with a suitable blocking solution containing, e.g., skim milk, powdered milk, BSA, gelatin, detergent, or other suitable blocking agents, to prevent nonspecific binding during subsequent steps.

Plates with biotinylated antigen are then incubated for a suitable amount of time for antigen to bind or attach to the surface. Biotinylated antigen in a Sigma SA plate is incubated at room temperature for 30 minutes. Excess biotinylated antigen solution is then removed from the plate. In this embodiment, blocking is not necessary because Sigma SA plates are pre-blocked.

In another embodiment using Costar® Universal-BIND™ plates, antigen is passively adsorbed overnight at 4 degrees C. in 1×PBS pH 7.4, 0.05% azide. Generally, if Costar® Universal-BIND™ plates are used, the initial concentration of antigen is a somewhat higher concentration, preferably 2-4 ug/ml. The next morning, excess antigen solution is removed from Costar® Universal-BIND™ plate or plates, preferably by “flicking”, and each plate is exposed to UV light at 365 nm for four (4) minutes. Each plate is then blocked with 1% skim milk/1×PBS pH 7.4 at 100 ul of blocking solution per well, for 30 minutes.

After incubation with antigen and removal of excess antigen solution, and blocking, if necessary, plates are washed four times (4×) with tap water. Plates may be washed by hand, or a microplate washer or other suitable washing tool may be used.

### Reference Antibody Dilution and Distribution

A reference antibody that recognizes and binds to the antigen is then added. The reference antibody is preferably a monoclonal antibody, but can alternatively be polyclonal antibodies, natural ligands or soluble receptors, antibody fragments or small molecules.

A solution of reference antibody, also known as anti-antigen antibody, at an initial concentration, preferably about 1 μg/ml, is diluted in a series of stepwise dilutions. Diluted samples are placed on surfaces such as in the wells of a microtiter plate, and replicates of each sample are also distributed on surfaces. Serial dilutions of reference antibody are distributed across the rows of a 96-well plate. Specifically, each reference antibody dilution is placed in a row, with replicate samples placed in each column of the row. In a 96-well plate, a different dilution of reference antibody is placed in each row, with replicates of each dilution placed in each column across each row starting at an initial concentration of about 1 μg/ml progressively and diluted 1:2 seven times for a series of seven wells. An ending concentration of about 30 ng/ml is used as the standard solution series. Solutions of reference antibody are incubated with bound antigen under suitable conditions determined by the materials and reagents being used, preferably about 24 hours at room temperature. One of skill in the art can determine whether incubation for longer or shorter times, or at higher or lower temperatures would be suitable for a particular embodiment.

Optional Step: Incubation with shaking. If desired, the plate may be tightly wrapped and incubation of the reference antibody with bound antigen may be carried out with shaking to promote mixing and more efficient binding. Plates containing reference antibody and bound antigen may be incubated overnight with shaking, for example as provided by a Lab Line Microplate Shaker at setting 3.

### Add Detection Antibody

Plates are washed to remove unbound reference antibody, preferably about five times (5×) with water. Next, a labeled detection antibody that recognizes and binds to the reference antibody is added, and the solution is incubated to permit binding of the detection antibody to the reference antibody. The detection antibody may be polyclonal or monoclonal. The detection antibody may be labeled in any manner that allows detection of antibody bound to the reference antibody. The label may be an enzymatic label such as alkaline phosphatase or horseradish peroxidase (HRP), or a non-enzymatic label such as biotin or digoxygenin, or may be a radioactive label such as ³²P, ³H, or ¹⁴C, or may be any other label suitable for the assay based on reagents, materials, and detection methods available.

Following labeling, 50 μl of goat anti-Human IgG Fe HRP polyclonal antibody (Pierce Chemical Co, Rockford Ill., catalog number 31416) at a concentration of 0.5 μg/ml in 1% skim milk, 1×PBS pH 7.4 is added to each well of a microtiter plate. The plate is then incubated for 1 hr at room temperature.

Excess solution containing detection antibody is removed, and plates are washed with water repeatedly, preferably at least five times, in order to remove all unbound detection antibody.

### Measurement of Bound Detection Antibody

The amount of detection antibody bound to reference antibody is determined by using the appropriate method for measuring and quantifying the amount of label present. Depending on the label chosen, methods of measuring may include measuring enzymatic activity against added substrate, measuring binding to a detectable binding partner (e.g., for biotin) scintillation counting to measure radioactivity, or any other suitable method to be determined by one of skill in the relevant art.

In the embodiment described above using goat anti-Human IgG Fc HRP polyclonal antibody as the detection antibody, 50 ul of the chromogenic HRP substrate tetramethylbenzidine (TMB) is added to each well. The substrate solution is incubated for about 30 minutes at room temperature. The HRP/TMB reaction is stopped by adding 50 ul of 1M phosphoric acid to each well.

### Quantification

The amount of bound label is then quantified by the appropriate method, such as spectrophotometric measurement of formation of reaction products or binding complexes, or calculation of the amount of radioactive label detected. Under the conditions disclosed here, the amount of label measured in this step is a measure of the amount of labeled detection antibody bound to the reference antibody.

In the embodiment described above using goat anti-Human IgG Fc HRP polyclonal antibody and TMB substrate, the amount of detection antibody bound to reference antibody is quantified by reading the absorbance (optical density or “OD”) at 450 nm of each well of the plate.

### Data Analysis to Determine Optimal Antigen Concentration

A known reference antibody concentration is chosen, and the results from wells having the chosen antibody concentration and different amounts of antigen are examined. The antigen concentration that produces the desired signal strength, or standard signal, is chosen as the optimal antigen concentration for subsequent experiments. The standard signal may be empirically determined according to the conditions and materials used in a particular embodiment, because the standard signal will serve as a reference point for comparing signals from other reactions. For a detection method that produces a chromogenic product, a desirable standard signal is one that falls within the most dynamic region of the ELISA reader or other detector and may be an optical density (OD) of between about 0.4 and 1.6 OD units and for this system preferably about 1.0 OD units, although it is possible to achieve signals ranging from 0.2 to greater than 3.0 OD units. Any OD value may be chosen as the standard signal, although an OD value of about 1.0 OD units permits a accurate measurement of a range of test signals above and below 1.0 OD units, and further permits easy comparison with other test signals and reference signals. The concentration of antigen identified as the concentration that produces the standard signal will be used in subsequent experiments to screen and kinetically rank antibodies.

In a preferred embodiment using a 96-well plate, a reference antibody concentration of 100 ng/ml is chosen. It is possible, depending on the sensitivity and antibody concentrations employed in the system, to use other reference antibody concentrations. The signals from the detection antibody reaction in the wells in all columns of the row containing 100 ng/ml antibody are then examined to find the antigen concentration that produces an OD value of about 1.0. In the preferred embodiment described above using goat anti-Human IgG Fe HRP polyclonal antibody and TMB substrate, the wells in the row containing 100 ng/ml antibody are examined to determine which antigen concentration produces a reaction which, when absorbance is measured at 450 nm, has an OD value of about 1.0. This concentration of antigen will then be used for the subsequent experiments to screen and kinetically rank antibodies. A similar approach for identifying optimal antigen densities was used for the Luminex bead based system.

### Screening Antibodies Using Limiting Antigen Concentrations

Coat Surfaces at Optimized Antigen Concentration

The surface or surfaces being used to carry out antibody screening are coated with antigen at the optimal concentration as previously determined. In a preferred embodiment, the surfaces are wells of a 96-well streptavidin plate such as a Sigma SA plate, and biotinylated antigen at optimal concentration is added the wells. In a more preferred embodiment, 50 μl of antigen in a solution of 1% skim milk, 1×PBS pH 7.4, and plates are incubated for 30 minutes. In another preferred embodiment, unmodified antigen is added to Costar® Universal-BIND™ plates, and incubation and UV-mediated antigen binding are carried out according to manufacturer's instructions and/or standard protocols, as described above.

After incubation with antigen solution for a suitable amount of time, plates are washed to remove unbound antigen, preferably at least four times (4×).

### Addition of Test Antibodies to be Screened and Ranked

Antibodies to be screened and ranked by the limiting antigen assay are called test antibodies. Test antibodies may be recovered from the solution surrounding antibody-producing cells. Preferably, test antibodies are recovered from the media of antibody-producing B cell cultures, hybridoma supernatants, antibody or antibody fragments expressed from any type of cell, more preferably from the supernatant of B cell cultures. Solutions containing test antibodies, for example B cell culture supernatants, generally do not require additional processing; however, additional steps to concentrate, isolate, or purify test antibodies would also be compatible with the disclosed methods.

Each solution containing test antibodies is diluted to bring the concentration within a desirable range and samples are added to a surface having attached antigen. Typically, a desirable concentration range for test antibodies has a maximum concentration lower than the concentration of reference antibody used to select the optimal antigen concentration as described above. One aspect of the present invention provides that a test antibody would produce a signal higher than that of the reference antibody for the same antigen concentration if the test antibody (a) has a higher affinity for the antigen, or (b) has a similar affinity but is present in higher concentration than the reference antigen. Thus, when test antibodies are used at concentrations lower than the concentration of the reference antibody used to select the antigen concentration used in the screening assay, only a test antibody having higher affinity for the antigen would produce a higher signal than the reference antibody signal.

In one embodiment in which a reference antibody concentration of 100 ng/ml is used to select the optimal antigen concentration (as described above), B cell culture supernatants having an empirically determined test antibody concentration range of between about 20 ng/ml to 800 ng/ml are typically diluted ten-fold to produce a working assay test antibody concentration of between about 2 ng/ml to 80 ng/ml. Preferably, at least two duplicate samples of each diluted B cell culture supernatant are tested. Preferably, the diluted B cell culture supernatants are added to wells of a microtiter plate, where the wells are coated with antigen at an optimal concentration previously determined using antigen and a reference antibody.

A positive control should be included as part of the screening, wherein the reference antibody used to optimize the assay by determining optimal antigen concentration is diluted and reacted with the antigen. The positive control provides a set of measurements useful both as an internal control and also to compare with previous optimization results in order to confirm, assure, and demonstrate that results from a screening of test antibodies are comparable with the expected results of the positive control, and are consistent with previous optimization results.

In one embodiment, each B cell culture supernatant to be tested is diluted 1:10 in 1% skim milk/1×PBS pH 7.4 /0.05% azide, and 50 ul is added to each of two antigen-coated wells of a 96-well plate, such that 48 different samples are present in each 96-well plate. A positive control comprising a dilution series of the reference antibody is preferably added to wells of about one-half a 96-well plate, to provide confirmation and to demonstrate that results of the screening of test antibodies in B cell culture supernatants run in parallel with the positive control are internally consistent and also consistent with previous optimization results.

Test antibodies are incubated with antigen under suitable conditions. Reference antibodies used as positive controls are incubated in parallel under the same conditions. In one preferred embodiment, plates are wrapped tightly, for example with plastic wrap or paraffin film, and incubated with shaking for 24 hours at room temperature.

### Add Detection Antibody to Test Antibodies

Plates are washed to remove unbound test antibodies, preferably about five times (5×) with water. Next, a labeled detection antibody that recognizes and binds to the test antibody is added, and the solution is incubated to permit binding of the detection antibody to the test antibody. Detection antibody is also added to the positive control, to confirm the interaction between the reference antibody and detection antibody. The detection antibody may be polyclonal or monoclonal. The detection antibody may be labeled in any matter that allows detection of antibody bound to the reference antibody. The label may be an enzymatic label such as alkaline phosphatase or horseradish peroxidase (HRP), or a non-enzymatic label such as biotin or digoxygenin, or a radioactive label such as ³²P, ³H, or ¹⁴C, or fluorescence, or it may be any other label suitable for the assay based on reagents, materials, and detection methods available.

In one embodiment, using human test antibodies, 50 μl of goat anti-Human IgG Fc HRP polyclonal antibody (Pierce Chemical Co, Rockford Ill., catalog number 31416) at a concentration of 0.5 μg/ml in 1% skim milk, 1×PBS pH 7.4 is added to each well of microtiter plates containing test antibodies and reference antibodies (as a positive control). The plate is then incubated for 1 hr at room temperature.

Excess solution containing detection antibody is removed, and plates are washed with water repeatedly, preferably at least five times, in order to remove all unbound detection antibody.

### Measurement of Bound Detection Antibody

The amount of detection antibody bound to test antibody (and bound to reference antibody of the control) is determined by using the appropriate method for measuring and quantifying the amount of label present. Depending on the label chosen, methods of measuring may include measuring enzymatic activity against added substrate, measuring binding to a detectable binding partner (e.g., for biotin) scintillation counting to measure radioactivity, or any other suitable method to be determined by one of skill in the relevant art.

In the method described above, using goat anti-Human IgG Fc HRP polyclonal antibody as the detection antibody, 50 μl of the chromogenic HRP substrate tetramethylbenzidine (TMB) is added to each well. The antibody-substrate solution is incubated for about 30 minutes at room temperature. The HRP/TMB reaction is stopped by adding 50 μl of 1M phosphoric acid to each well.

### Quantification

The amount of bound label is then quantified by the appropriate method, such as the spectrophotometric measurement of formation of reaction products or binding complexes, or calculation of the amount of radioactive label detected. In accordance with one aspect of the present invention, the amount of label provides a measure of the amount of labeled detection antibody bound to the test antibody (or, in the positive control, bound to the reference antibody). In accordance with another aspect of the present invention, the amount of label provides a measure of the amount of test antibody bound to antigen. Thus, detecting and quantifying the amount of label provides a means of measuring the binding of test antibody to the test antigen. By comparing the standard signal with the signal that quantifies the amount of test antibody bound to antigen, it is possible to identify test antibodies with higher affinities by searching for test antibodies which give a higher signal than the reference.

In the method described above using goat anti-Human IgG Fc HRP polyclonal antibody and TMB substrate, the amount of detection antibody bound to test antibody (and reference antibody in the positive control) is quantified by reading the absorbance (optical density, OD) at 450 nm of each well of each plate.

### Data Analysis to Identify and Rank Antibodies of Interest

The results from each test antibody are averaged and the standard range is determined. In a preferred embodiment wherein two samples of each test antibody are assayed using a HRP-labeled detection antibody, OD values at 450 nm are averaged and the standard deviation is calculated. The average OD values of test antibodies are compared against the OD value of the standard signal. Values from the positive control assays are also calculated and examined for reliability of the assay.

Test antibodies are kinetically ranked by considering the average OD value and the range of the OD's between replicates. The average OD value provides a measure of the affinity of the test antibody for the antigen, where affinity is determined by comparison with the standard signal, or the OD value of the reference antibody in the positive control. The range provides a measure of reliability of the assay, where a narrow range indicates that the OD values are likely to be accurate measurements of the amount of test antibody bound to the antigen, and a wide range indicates that the OD values may not be accurate measurements of binding. Acceptable standard deviations are typically OD's of between 5-15% of each other. Test antibodies giving the highest OD values, where the standard deviation of the average value is low, are given the highest kinetic ranking.

In one embodiment, wherein the standard signal is 1.0 OD units, any test antibody with both an average OD of greater than 1.0 OD units, and an acceptably low standard deviation, is considered to have a higher affinity for the antigen than the affinity of the reference antibody.

In another embodiment, Luminex based assays using differentially antigen coated beads were used. In this assay antibodies were ranked based on how they bound antigen at higher then at lower antigen densities.

### EXAMPLES

### Example 1

### Assay of Epitope Recognition Properties

Generation and Preliminary Characterization of Antibodies.

Hybridoma supernatants containing antigen-specific human IgG monoclonal antibodies used for binning were collected from cultured hybridoma cells that had been transferred from fusion plates to 24-well plates. Supernatant was collected from 24-well plates for binning analysis. Antibodies specific for the antigen of interest were selected by hybridoma screening, using ELISA screening against their antigens. Antibodies positive for binding to the antigen were ranked by their binding affinity through a combination of a 96-well plate affinity ranking method and BlAcore affinity measurement. Antibodies with high affinity for the antigen of interest were selected for epitope binning. These antibodies will be used as the reference and probe test antibodies in the assay.

Assay Using Luminex Beads

First, the concentration of mouse anti-human IgG (mxhIgG) monoclonal antibodies used as capture antibody to capture the reference antibody was measured, and mxhIgG antibodies were dialyzed in PBS to remove azides or other preservatives that could interfere with the coupling process. Then the mxhIgG antibodies were coupled to Luminex beads (Luminex 100 System, Luminex Corp., Austin Tex.) according to manufacturer's instructions in the Luminex User Manual, pages 75-76. Briefly, mxhIgG capture antibody at 50 μg/ml in 500 μl PBS was combined with beads at 1.25×10⁷ beads/ml in 300 μl. After coupling, beads were counted using a hemocytometer and the concentration was adjusted to 1×10⁷ beads/ml.

The antigen-specific antibodies were collected and screened as described above, and their concentrations were determined. Up to 100 antibodies were selected for epitope binning. The antibodies were diluted according to the following formula for linking the antibodies to up to 100 uniquely labelled beads to form labelled reference antibodies:

Total volume of the samples in each tube: Vt=(n+1)×100 μl +150μl, where n=total number of samples including controls.

Volume of individual sample needed for dilution: Vs=C×Vt/Cs, Cs=IgG concentration of each sample. C=0.2−0.5 μg/ml.

Samples were prepared according to the above formula, and 150 μl of each diluted sample containing a reference antibody was aliquotted into a well of a 96-well plate. Additional aliquots were retained for use as a probe antibody at a later stage in the assay. The stock of mxhIgG-coupled beads was vortexed and diluted to a concentration of 2500 of each bead per well or 0.5×10⁵/ml. The reference antibodies were incubated with mxhIgG-coupled beads on a shaker in the dark at room temperature overnight.

A 96-well filter plate was pre-wetted by adding 200 μl wash buffer and aspirating. Following overnight incubation, beads (now with reference antibodies bound to mxhIgG bound to beads) were pooled, and 100 μl was aliquotted into each well of a 96-well microtiter filter plate at a concentration of 2000 beads per well. The total number of aliquots of beads was twice the number of samples to be tested, thereby permitting parallel experiments with and without antigen. Buffer was immediately aspirated to remove any unbound reference antibody, and beads were washed three times.

Antigen was added (50 μl) to one set of samples; and beads were incubated with antigen at a concentration of 1 μg/ml for one hour. A buffer control is added to the other set of samples, to provide a negative control without antigen.

All antibodies being used as probe antibodies were then added to all samples (with antigen, and without antigen). In this experiment, each antibody being used as a reference antibody was also used as a probe antibody, in order to test all combinations. The probe antibody should be taken from the same diluted solution as the reference antibody, to ensure that the antibody is used at the same concentration. Probe antibody (50 μl/well) was added to all samples and mixtures were incubated in the dark for 2 hours at room temperature on a shaker. Samples were washed three times to remove unbound probe antibody.

Detection antibody: Biotinylated mxhIgG (50 μl/well) was added at a 1:500 dilution, and the mixture was incubated in the dark for 1 hour on a shaker. Beads were washed three times to remove unbound Biotinylated mxhIgG. Streptavidin-PE at 1:500 dilution was added, 50 μl/well. The mixture was incubated in the dark for 15 minutes at room temperature on a shaker, and then washed three times to remove unbound components.

In accordance with manufacturer's instructions, the Luminex 100 and XYP base were warmed up using Luminex software. A new session was initiated, and the number of samples and the designation numbers of the beads used in the assay were entered.

Beads in each well were resuspended in 80 μl dilution buffer. The 96-well plate was placed in the Luminex based and the fluorescence emission spectrum of each well was read and recorded.

Optimization of Assay

To optimize the assay, the Luminex User's Manual Version 1.0 was initially used for guidance regarding the concentrations of beads, antibodies, and incubation times. It was determined empirically that a longer incubation time provided assured binding saturation and was more suitable for the nanogram antibody concentrations used in the assay.

### Example 2

### Analysis of a Single Data Set: ANTIGEN14 Antibodies

Data Input

Antibodies were assayed as described in Example 1, and results were collected. Input files consisted of input matrices shown in FIG. 8A (antigen present) and FIG. 8B (antigen absent) for a data set corresponding to a single experiment for the ANTIGEN14 target.

Normalization of ANTIGEN14 Target Data

First, the matrix corresponding to the experiment without antigen (negative control, FIG. 8B) experiment was subtracted from the matrix corresponding to the experiment with antigen (FIG. 8A), to eliminate the amount of background signal due to nonspecific binding of the labelled antibody. The difference between the two matrices is shown in FIG. 9. The column corresponding to antibody 2.42 has unusually large values both on and off the diagonal and is flagged and treated separately in the data analysis as described above.

Row Normalization

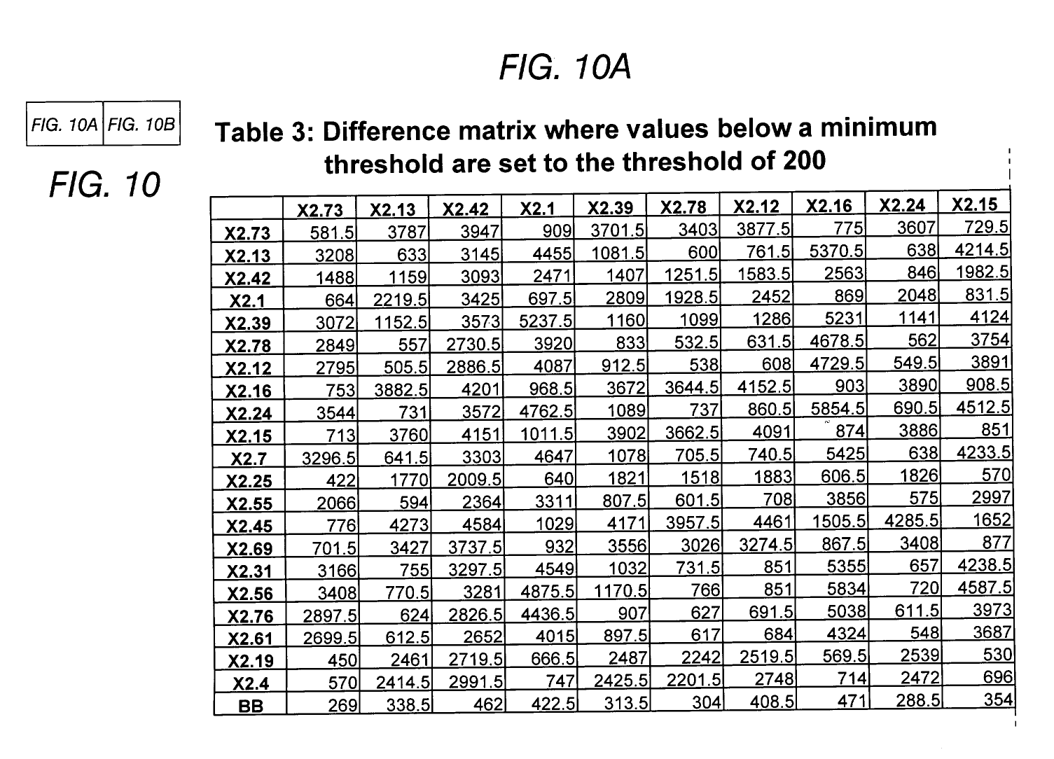

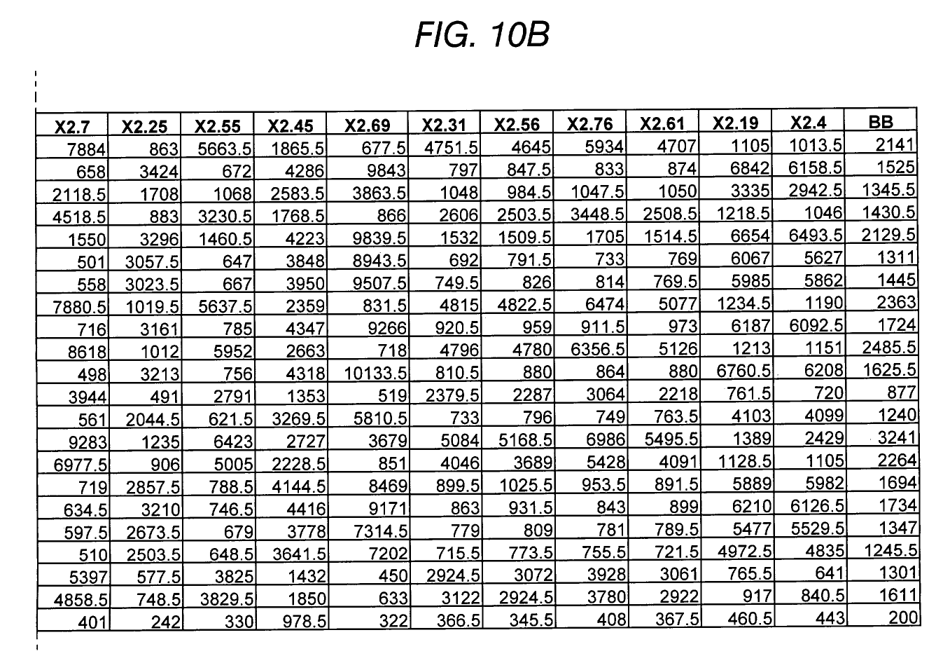

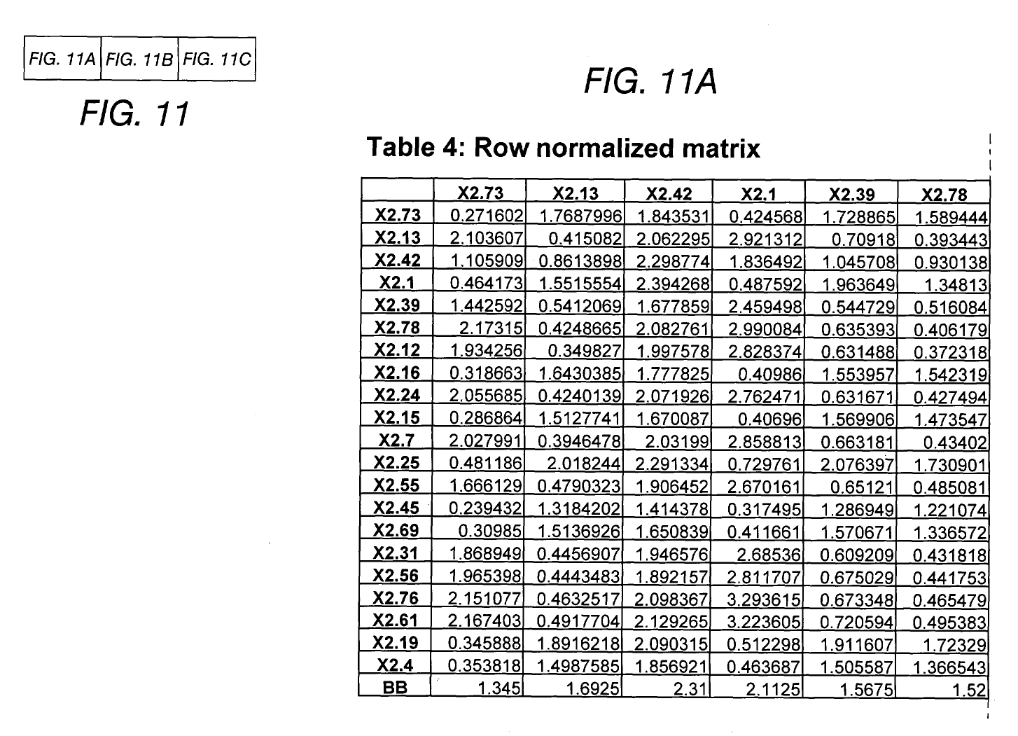

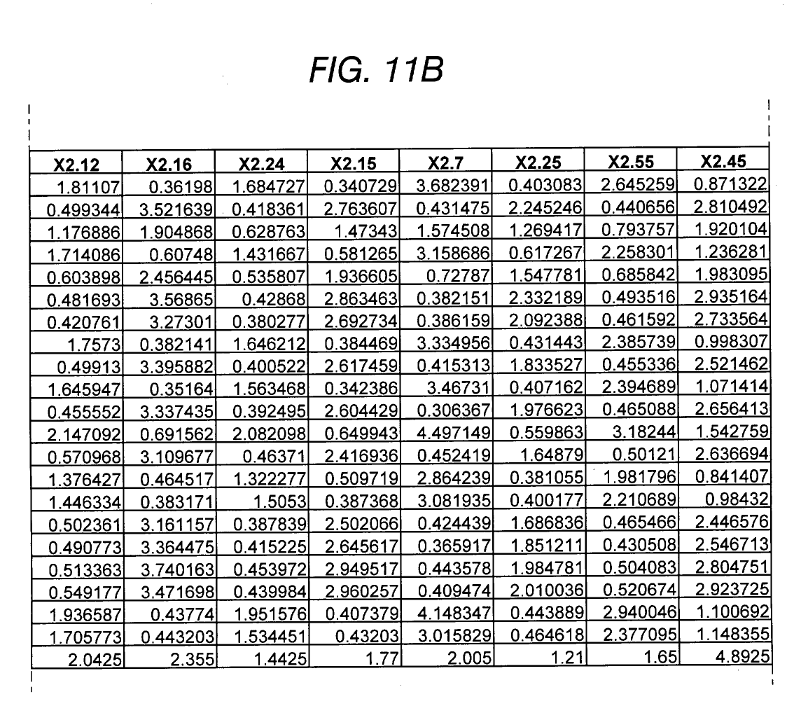

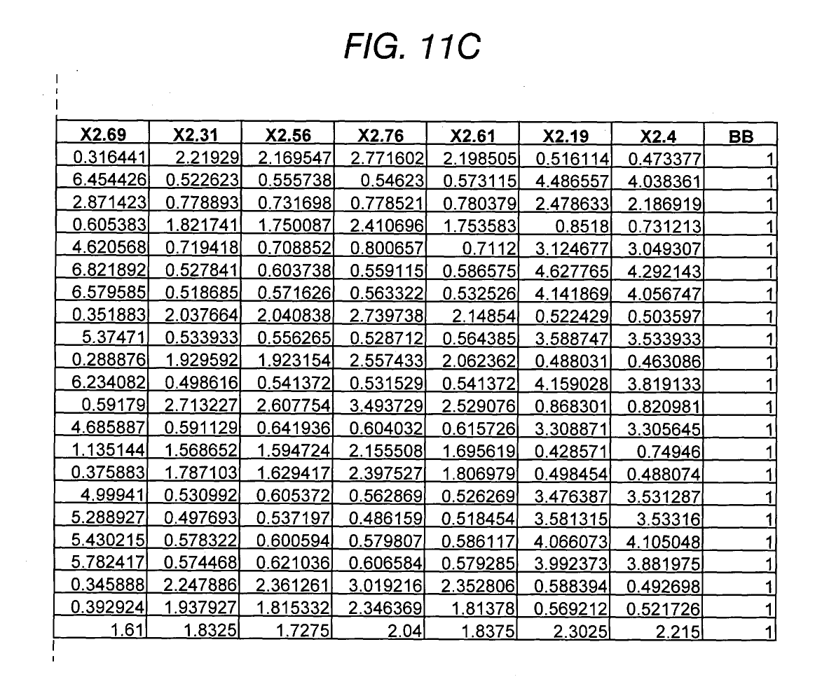

The difference matrix was adjusted by setting values below the user-defined threshold value of 200 to this threshold value as shown in FIG. 10. This adjustment was done to prevent significant artificial inflation of low signal values in subsequent normalization steps (as described above). The intensities of each row in the matrix were then normalized by dividing each row value by the row value corresponding to blocking buffer (FIG. 11). This adjusts for the well-to-well intensity variation as discussed above and illustrated in FIG. 2A.

Column Normalization

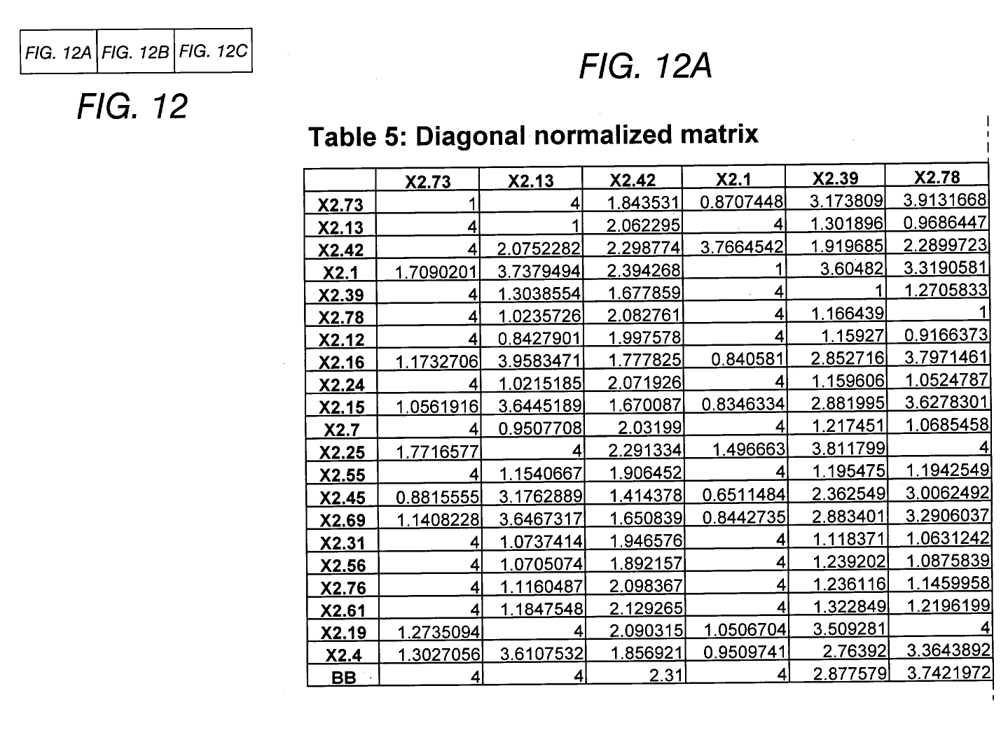

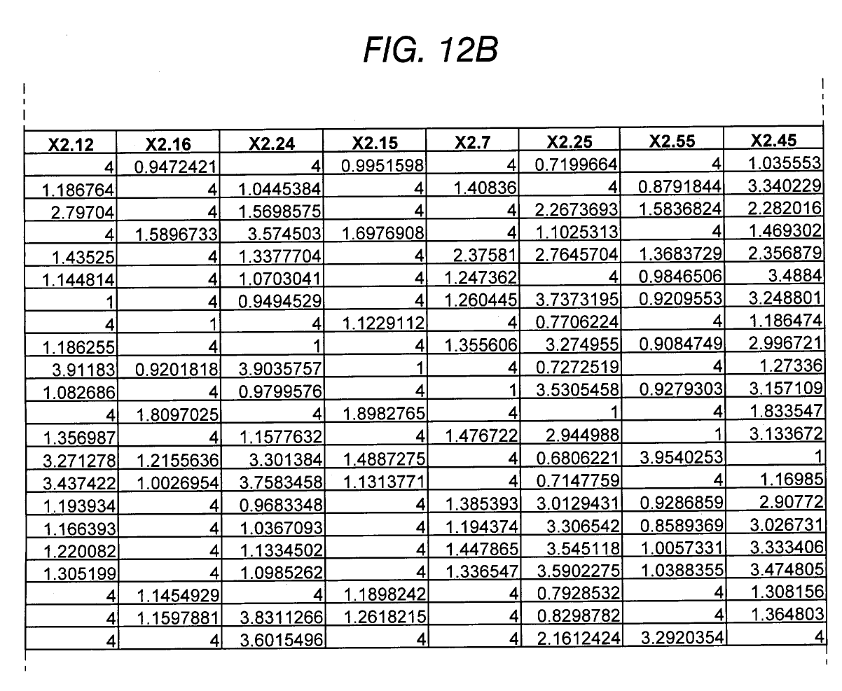

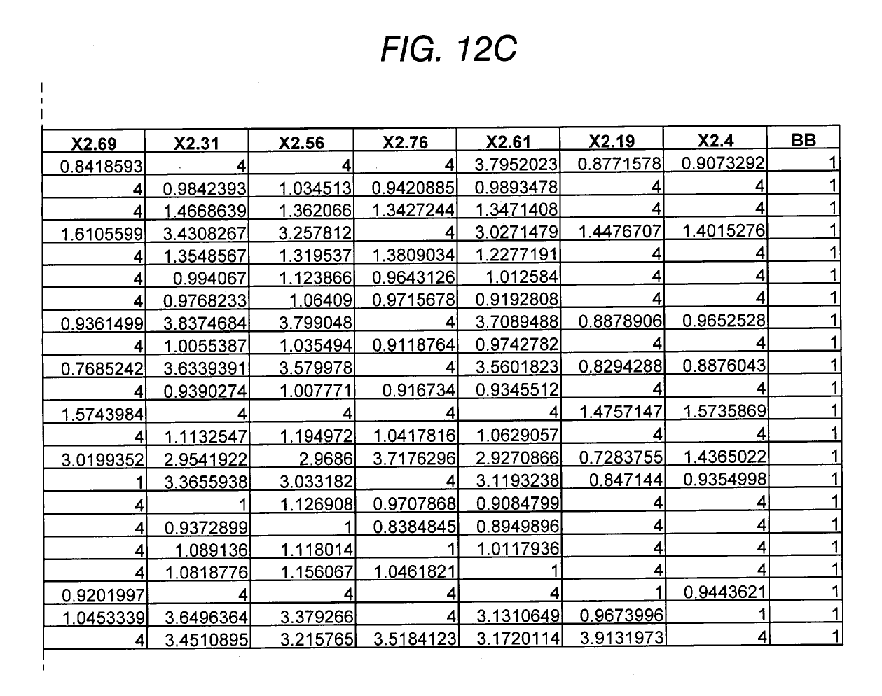

All columns except the one corresponding to antibody 2.42 were column-normalized as described above and are shown in FIG. 12.

Dissimilarity Matrix

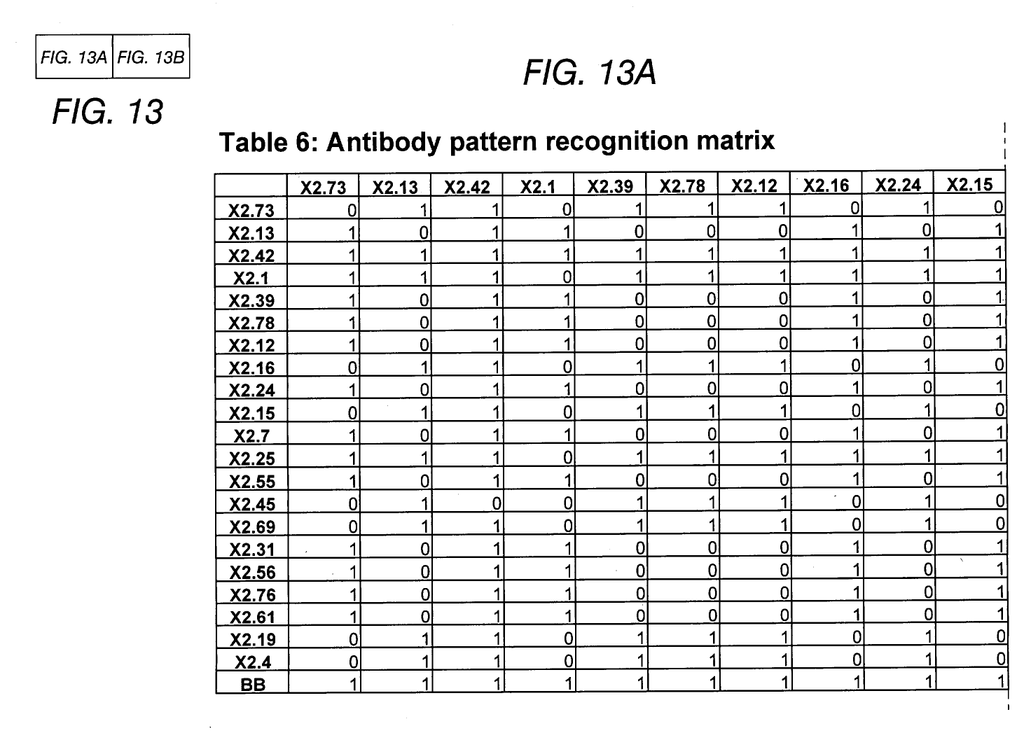

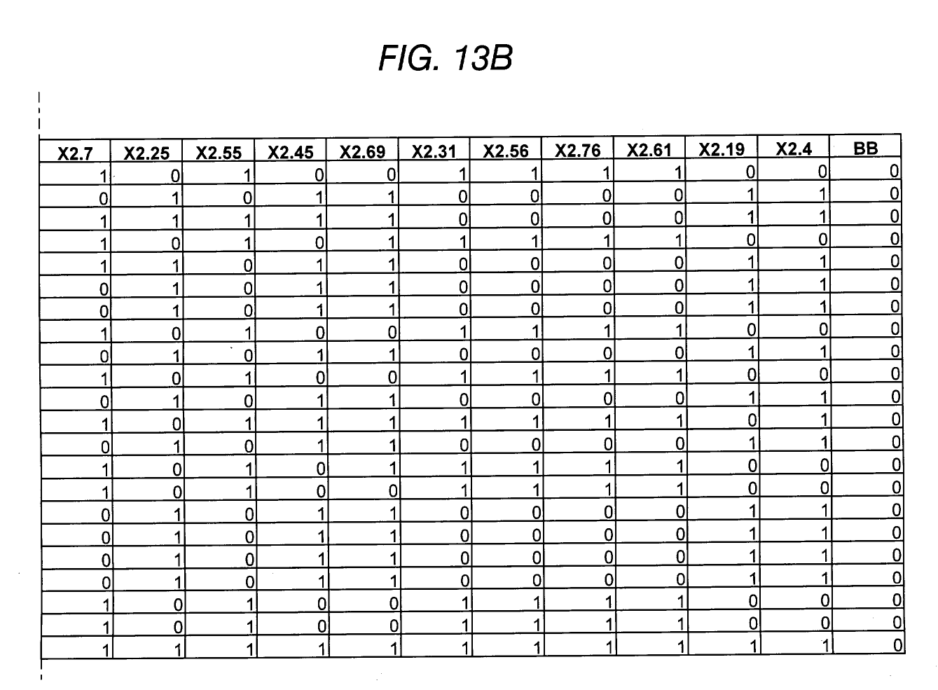

A dissimilarity (or distance) matrix was generated in a multistep procedure. First, intensity values below the user-defined threshold (set to two times the diagonal intensity values) were set to zero and the remaining values were set to one (FIG. 13). This means that intensity values that are less than twice the intensity value of the diagonal value are considered low enough to represent competition for the same epitope by the antibody pair. The dissimilarity matrix is generated from the matrix of zeroes and ones by setting the entry in row i and column j to the fraction of the positions at which two rows, i and j differ. FIG. 14 shows the number of positions (out of 22 total) at which the patterns for any two antibodies differed for the set of antibodies generated against the ANTIGEN14 target.

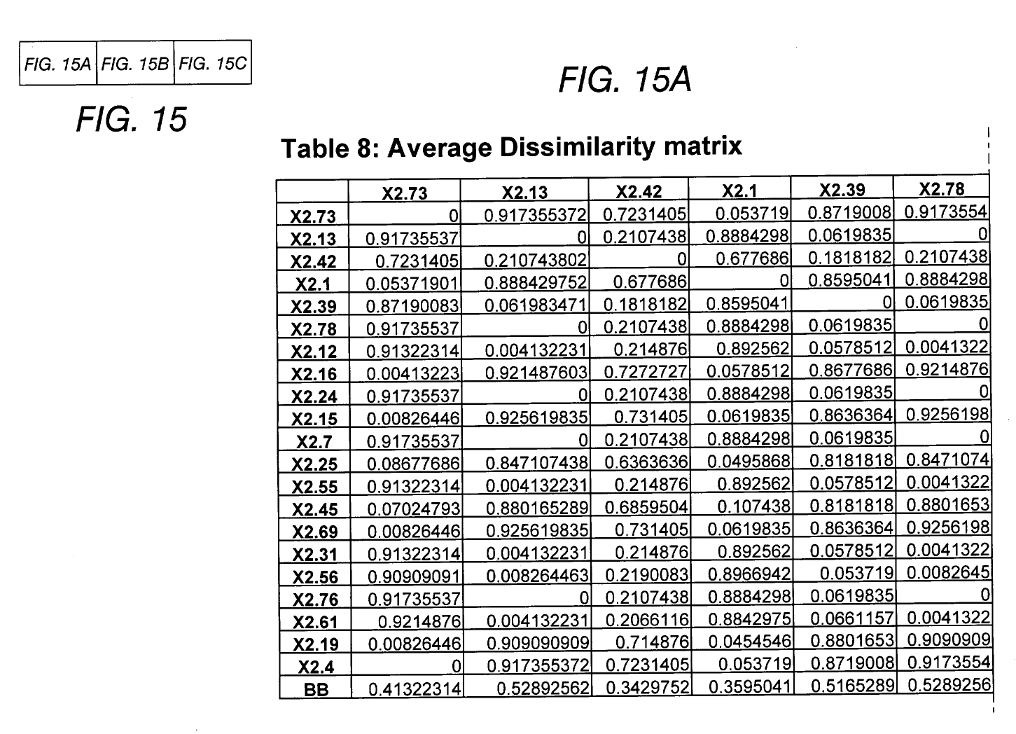

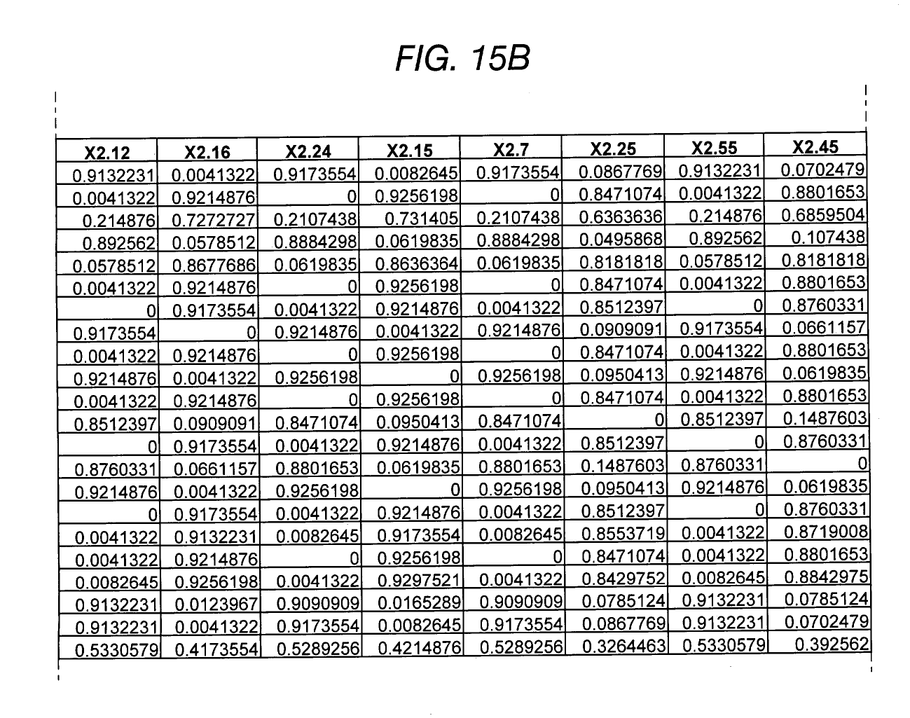

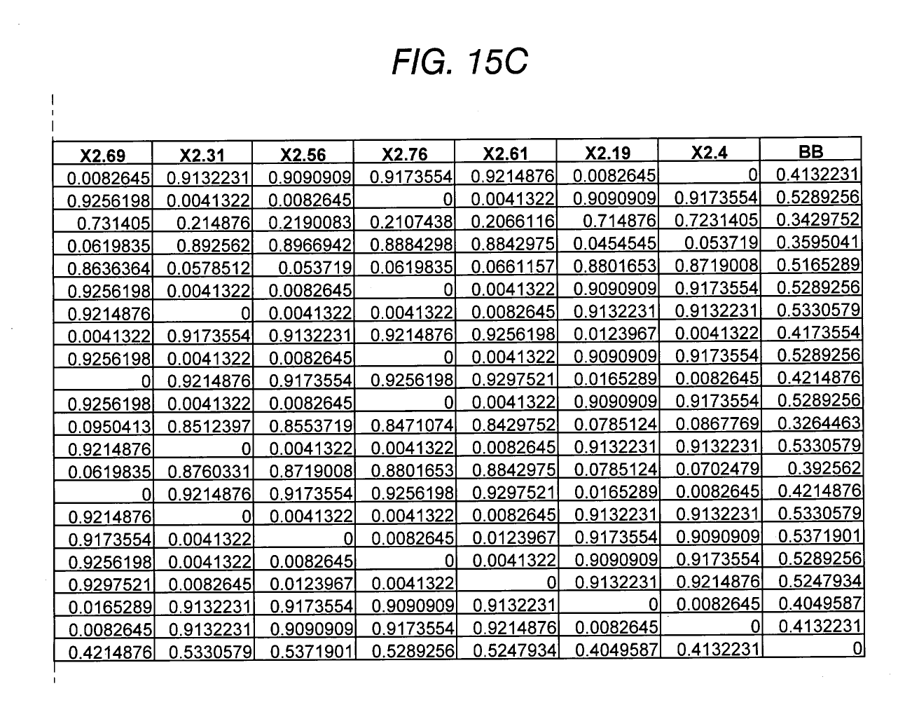

A dissimilarity matrix was generated from the matrix of zeroes and ones generated from each of several threshold values ranging from 1.5 to 2.5 (times the values of the diagonals), in increments of 0.1. The average of these dissimilarity matrices was computed (FIG. 15) and used as input to the clustering algorithm. The significance of taking the average of several dissimilarity matrices is illustrated in FIG. 4. FIG. 4 shows the fraction of dissimilarities for a pair of antibodies (2.1 and 2.25) as a function of the threshold value for threshold values ranging from 1.5 to 2.5. As the threshold value changed from 1.8 and 1.9 the amount of dissimilarity between the signal patterns for the two antibodies changed substantially from 0% to nearly 15%. This figure shows how the amount of dissimilarity between the signal patterns for a pair of antibodies may be sensitive to one particular choice of cutoff value, as it can vary substantially for different threshold values.

### Clustering

Hierarchical Clustering

Using the Agglomerative Nesting Subroutine in SPLUS 2000 statistical analysis software, antibodies were grouped (or clustered) using the average dissimilarity matrix described above as input. In this algorithm, antibodies were forced into a strict hierarchy of nested subsets. The pair of antibodies with the smallest corresponding dissimilarity value in the entire matrix is grouped together first. Then, the pair of antibodies, or antibody-cluster, with the second smallest dissimilarity (or average dissimilarity) value is grouped together next. This process was iteratively repeated until one cluster remained.

Visualizing Clusters in Dendrograms

The dendrogram calculated for the ANTIGEN14 target is shown in FIG. 5. The length (or height) of the branches connecting two antibodies is inversely proportional to the degree of similarity between the antibodies it binds. This dendrogram shows that there were two very distinct epitopes recognized by these antibodies. One epitope was recognized by antibodies 2.73, 2.4, 2.16, 2.15, 2.69, 2.19, 2.45, 2.1, and 2.25. A different epitope was recognized by antibodies 2.13, 2.78, 2.24, 2.7, 2.76, 2.61, 2.12, 2.55, 2.31, 2.56, and 2.39. Antibody 2.42 does not have a pattern that was very similar to any other antibody but had some noticeable similarity to the second cluster, indicating that it may recognize yet a third epitope which partially overlaps with the second epitope.

Visualizing Clusters in Matrices

Clustering of these antibodies can also be seen in FIG. 16 and FIG. 17. In FIG. 16 the rows and columns of the dissimilarity matrix were rearranged according to the order of the “leaves” or leaves on the dendrogram and the individual cells were visually coded according to the degree of dissimilarity. Cells that have darker shading correspond to antibody pairs that were very similar (less than 10% dissimilar). Cells that are unshaded correspond to those antibodies that were fairly similar (between 10% and 25% dissimilar). Cells that have lighter shading correspond to antibody pairs that were more than 25% dissimilar. The darker shaded blocks correspond to different clusters of antibodies. Excluding the blocking buffer, there appeared to be two, or possibly three, blocks corresponding to the groups of antibodies mentioned above. FIG. 16 also shows that, allowing for a slightly higher tolerance for dissimilarity, Antibody 2.42 can be considered a member of the second cluster.

In FIG. 17, the rows and columns of the normalized intensity matrix were rearranged according to the order of the leaves on the dendrogram and the individual cells were visually coded according to their normalized intensity values. Cells that are have darker shading correspond to antibody pairs that had a high intensity (at least 2.5 times greater than the background). Cells that are unshaded had an intensity between 1.5 and 2.5 times the background. Cells that have lighter shading correspond to intensities that were less than 1.5 times the background. When comparing the visual markings of the rows of this matrix, two very distinct patterns emerged corresponding to the two epitopes shown above. Furthermore, note that the visual coding is very symmetric with respect to the diagonal. This shows that there was a high level of self-consistency for the data with regard to revealing whether two antibodies compete for the same epitope. The reason is that if antibody A and antibody B compete for the same epitope, then the intensity should be low both when antibody A is the primary antibody and antibody B is the secondary antibody, as well as when antibody B is the primary antibody and antibody B is the secondary antibody. Therefore, the intensity for the cell of the i^{th} row and j^{th} column as well that for the j^{th} row and i^{th} column should both be low. Likewise, if these two antibodies recognized different epitopes, then both corresponding intensities should have been high. Out of the approximately 200 pairs of cells, for only one pair did one member of the pair have an intensity below 1.5 while the other member had an intensity above 2.5. The level of self-consistency of the resulting normalized matrices produced by the algorithm provided a measure of the reliability of both the data generated as well as the algorithm's analysis of the data. The high level of self-consistency for the ANTIGEN14 data set (over 99%) suggests that one can trust the results of the algorithm for this data set with a high level of confidence.

### Example 3

### Analysis of Multiple Data Sets: ANTIGEN39

When there are input data sets for more than one experiment, normalized intensity matrices are first generated as described above for each individual experiment. Normalized values above a threshold value (typically set to 4) are set to the corresponding threshold value. This prevents any single normalized intensity value from having too much influence on the average value for that antibody pair. A single normalized matrix is generated from the individual normalized matrices by taking the average of the normalized intensity values over all experiments for each antibody pair for which there is data. Antibody pairs with no corresponding intensity values are flagged. The generation of the dissimilarity matrix is as described above with the exception that the fraction of the positions at which two rows, i and j differ only considers the number of positions for which both rows have an intensity value. If the two rows have no such positions, then the dissimilarity value is set arbitrarily high and flagged.

Five experiments were conducted using ANTIGEN39 antibodies, using methods described in Examples 1 and 2, and throughout the description. The clustering results for the five input data sets of ANTIGEN39 antibodies are summarized in FIG. 6A, FIG. 18, FIG. 19, and FIG. 30. The results show that there were a large number of clusters of varying degree of similarity. This suggests there were several different epitopes, some of which may overlap. For example, the cluster containing antibodies 1.17, 1.55, 1.16, 1.11, and 1.12 and the cluster containing 1.21, 2.12, 2.38, 2.35, and 2.1 are fairly closely related (each antibody pair with the exception of 2.35 and 1.11 being no more than 25% different). This high degree of similarity across the two clusters suggests that the two different epitopes may have a high degree of similarity

In order to test the algorithm's ability to produce consistent clustering results, the five data sets were also independently clustered. The clustering results for the different experiments are summarized in FIGS. 6B-6F and in FIGS. 20-30. FIG. 30 summarizes the clusters for each of the individual data sets and for the combined data set with all of the antibodies for the five experiments. FIG. 6B shows the dendrogram for the ANTIGEN39 antibodies for Experiment 1: Antibodies 1.12, 1.63, 1.17, 1.55, and 2.12 consistently clustered together in this experiment as well as in other experiments as do antibodies 1.46, 1.31, 2.17, and 1.29. FIG. 6C shows the dendrogram for the ANTIGEN39 antibodies for Experiment 2: Antibodies 1.57 and 1.61 consistently clustered together in this experiment as well as in other experiments.

FIG. 6D shows the dendrogram for the ANTIGEN39 antibodies for Experiment 3: Antibodies 1.55, 1.12, 1.17, 2.12, 1.11, and 1.21 consistently clustered together in this experiment as well as in other experiments. FIG. 6E shows the dendrogram for the ANTIGEN39 antibodies for experiment 4: Antibodies 1.17, 1.16, 1.55, 1.11, and 1.12 consistently clustered together in this experiment as well as in other experiments as do antibodies 1.31, 1.46, 1.65, and 1.29, as well as antibodies 1.57 and 1.61. FIG. 6F shows the dendrogram for the ANTIGEN39 antibodies for experiment 5: Antibodies 1.21, 1.12, 2.12, 2.38, 2.35, and 2.1 consistently clustered together in this experiment as well as in other experiments.

In general, the clustering algorithm produced consistent results both among the individual experiments and between the combined and individual data sets. Antibodies which cluster together or are in neighboring clusters for multiple individual data sets also cluster together or be in neighboring clusters for the combined data set. For example, the cells with lighter shading correspond to antibodies that consistently clustered together in the combined data set and in all of the data sets in which they were present (Experiments 1, 3, 4, and 5). These results indicate that the algorithm produces consistent clustering results both across multiple individual experiments and that it retains the consistency upon the merging of multiple data sets.

Finally, there is a high level of self-consistency for the data with regard to revealing whether or not two antibodies compete for the same epitope. The percent of antibody pairs for which the data consistently reveals whether or not they compete for the same epitope is summarized for each data set in Table 2, above. Table 2 (above) reveals that the consistency was nearly 90% for four out of the five individual data sets as well as for the combined data set.

### Example 4

### Analysis of a Small Set of IL-8 Human Monoclonal Antibodies Using the Competitive Pattern Recognition Data Analysis Process

A small set of well-characterized human monoclonal antibodies developed against IL-8, a proinflammatory mediator, was used to evaluate the program applying the CPR process. Previously, plate-based ELISAs had shown that antibodies within the set bound two different epitopes: HR26, a215, and D111 recognized one epitope, whereas K221 and a33 competed for a second epitope. Further analysis using epitope mapping studies showed that HR26, a809, and a928 bound to the same or overlapping epitopes, while a837 bound to a different epitope.

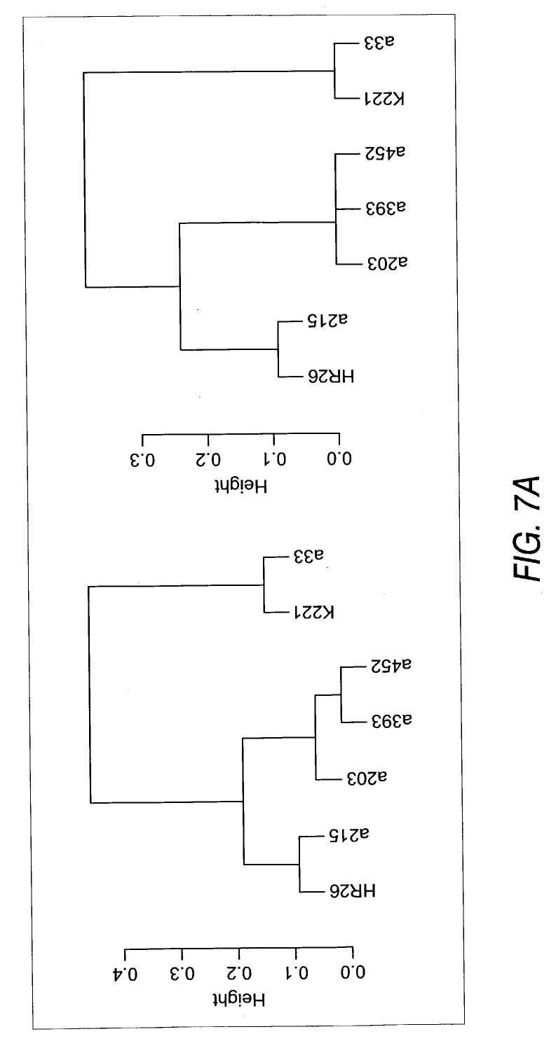

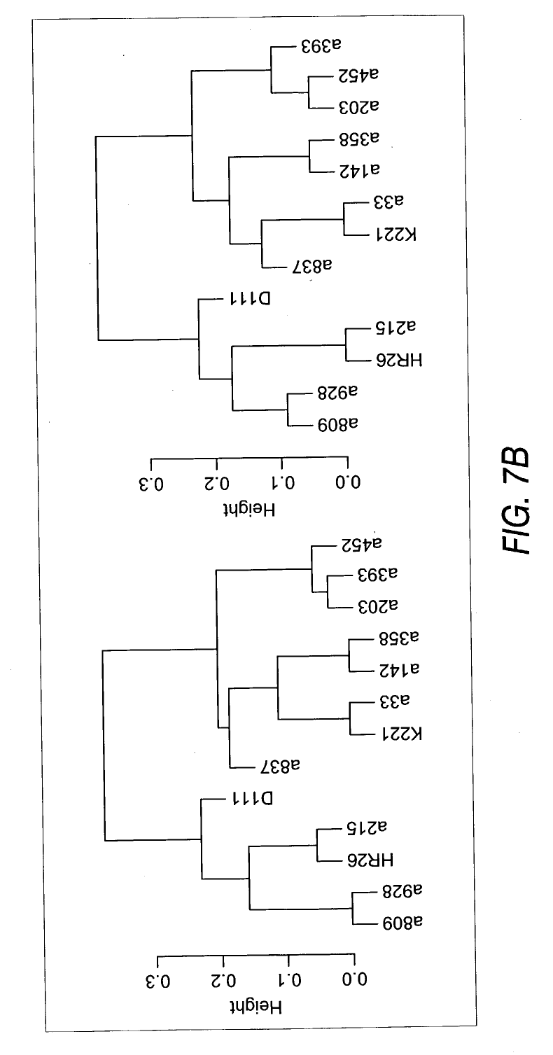

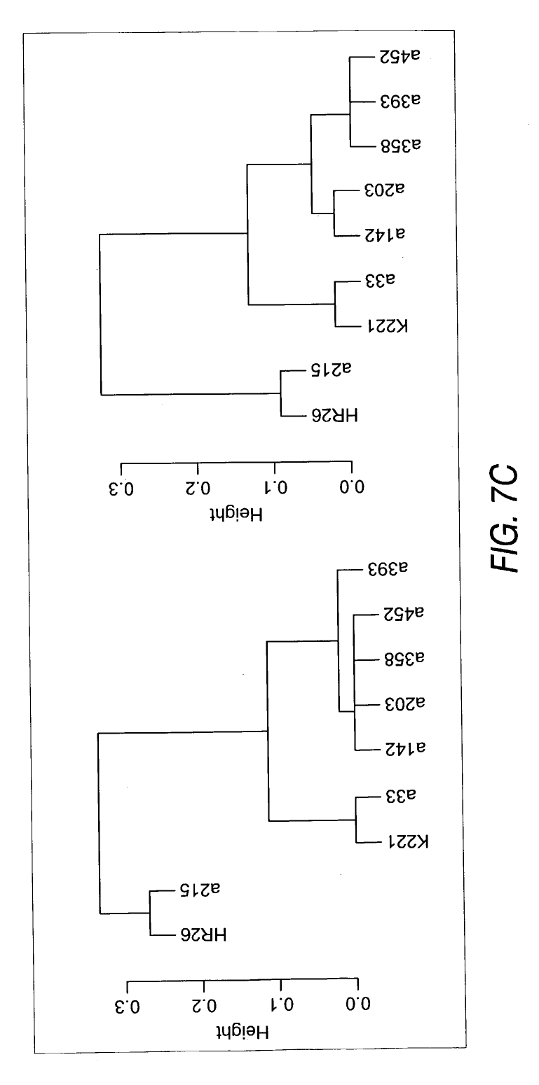

In a new experiment to determine whether the CPR process was capable of correctly clustering antibodies, the process was tested on a set of seven IL-8 antibodies, including some of the monoclonal antibodies listed above. The results are summarized in the dendrograms shown in FIG. 7A. The dendrogram on the left was generated by clustering columns, and the dendrogram on the right was generated by clustering rows of the background-normalized signal intensity matrix. Both dendrograms indicated that there were two epitopes for a dissimilarity cut-off of 0.25: one epitope recognized by HR26, a215, a203, a393, and a452, and a second epitope recognized by K221 and a33.

These results using the CPR process to cluster antibodies were consistent with the data from plate-based ELISA assays summarized above. The results obtained using the CPR process indicated that the target antigen appeared to have two distinct epitopes, confirming the results seen using plate-based ELISA assays. Using the CPR process for clustering indicated that HR26 and a215 clustered together, as did K221 and a33, again consistent with the results from plate-based ELISA assays.

The degree of similarity between the two dendrograms provided a measure of the self-consistency of the analyses performed by this process. Ideally, the two dendrograms (the one on the left generated by clustering columns and the one on the right generated by clustering rows) should have been identical for the following reason: if Antibody #1 and Antibody #2 compete for the same epitope, then the intensity should be low when Antibody #1 is the reference antibody and Antibody #2 is the probe antibody, as well as when Antibody #2 is the reference antibody and Antibody #1 is the probe antibody. Likewise, when the two antibodies bind to different epitopes, the intensities should be uniformly high. By this reasoning, the degree of similarity between two rows of the signal intensity matrix should be the same as between two columns of the similarity matrix. In the present example, the dendrograms on the left- and right-hand side of FIG. 7A are nearly identical. In each case, the same antibodies appeared in the two clusters. This high level of self-consistency between row and column clusterings suggested that the experimental protocol, together with the process, produces robust results.

### Example 5

### Analysis of Multiple Data Sets of IL-8 Antibodies Using the Competitive Pattern Recognition (CPR) Data Analysis Process

Multiple screening experiments using IL-8 antibodies were carried out, generating multiple data sets. Normalized intensity matrices were first generated as described above for the matrices for each individual experiment. Normalized values greater than a user-defined threshold value were set to the user-defined threshold value. High-intensity values were assigned to the threshold value to prevent any single intensity value from having too much weight when the average normalized intensity value was computed for that particular pair of antibodies in a subsequent step. The rows and columns of the average normalized intensity matrix corresponded to the set of “unique” antibodies identified using the methods of the present invention. These “unique” antibodies were identified from among all the antibodies used in all the experiments. The average intensity was computed for each cell in this matrix for which there was at least one intensity value. Cells corresponding to antibody pairs with no data were identified as missing data points. Generation of the dissimilarity matrix was as described above, except that the fraction was determined based on the number of positions at which two rows differed relative to the total number of positions for which both rows had intensity values. If the two rows had no common data, then the dissimilarity value for the corresponding cell was flagged and set arbitrarily high, so the corresponding antibodies would not be grouped together as an artifact.

The clustering results for a set of monoclonal antibodies from five overlapping sets of monoclonal antibodies are summarized in FIG. 7B and Table 4 (below). These dendrograms corroborate the results showing there are two different epitopes on the target antigen. The first epitope is defined by monoclonal antibodies a809, a928, HR26, a215, and D111 and the second epitope is defined by monoclonal antibodies a837, K221, a33, al42, and a358, a203, a393, and a452. The lengths of the branches connecting the clusters indicated that, whereas the first cluster was very different from the other two, the second and third clusters were similar to each other.

To test the capacity of the CPR process to produce consistent results across separate experiments, the five data sets were also independently clustered. The clustering results for the different experiments are summarized in the dendrograms shown in FIGS. 7A, 7B, and 7C. These dendrograms demonstrated that the CPR clustering process produced consistent results among the individual experiments and between combined and individual data sets. Each dendrogram had two major branches, indicating two epitopes. Antibodies that clustered together for multiple individual data sets also clustered together or were in neighboring clusters for the combined data set. As shown in Table 4, below, there were only two minor discrepancies in the clustering results across different experiments or between an individual experiment and the combined data set, where these discrepancies are indicated by bold type in Table 4. In a data set generated in Experiment 3, D111 clustered with antibodies a33 and K221, instead of HR26 and a215. In a data set generated in Experiment 4, antibodies a203, a393, and a452 appeared in the first cluster, whereas in another experiment (as well as in the combined data set), they appeared in a second cluster. This slight difference'is likely attributable to differences in individual antibody affinity between experiments in which the antibody is used as a probe antibody and experiments in which the same antibody is used as a reference antibody. Antibodies with lower affinity may have a reduced capacity to capture antigen out of the solution when used as a reference antibody. However, the overall similarity of the clustering results, as well as the grouping of the antigens, indicated that the process produced consistent clustering results that were in good agreement with results from other experiments across multiple individual experiments, and that the results remained consistent when multiple data sets were merged.

Finally, there was a high level of consistency in clustering results for each of these data sets when the process was used to cluster by rows and by columns, for the individual and combined data sets. The only discrepancy in the clustering results between row and column clusterings was with D111 in the third data set, in which it clustered with antibodies HR26 and a215 when row clustering was performed, whereas D111 clustered with antibodies a33 and K221 when column clustering was performed.

TABLE 4
Results of Clustering for Individual and Combined Data Sets

| | Expt1 | Expt1 | Expt2 | Expt2 | Expt3 | Expt3 | Expt4 | Expt4 | Expt5 | Expt5 | Comb | Comb |
|---|---|---|---|---|---|---|---|---|---|---|---|---|
| Cluster | Rows | Cols | Rows | Cols | Rows | Cols | Rows | Cols | Rows | Cols | Rows | Cols |
| 1 | a809 | a809 | D111 | D111 | D111 | HR26 | HR26 | HR26 | HR26 | HR26 | a809 | a809 |
| | a928 | a928 | HR26 | HR26 | HR26 | a215 | a215 | a215 | a215 | a215 | a928 | a928 |
| | HR26 | HR26 | a215 | a215 | a215 | | a203 | a203 | | | D111 | D111 |
| | | | | | | | a393 | a393 | | | HR26 | HR26 |
| | | | | | | | a452 | a452 | | | a215 | a215 |
| 2 | a837 | a837 | a33 | a33 | a33 | D111 | a33 | a33 | a33 | a33 | a837 | a837 |
| | K221 | K221 | K221 | K221 | K221 | a33 | K221 | K221 | K221 | K221 | a33 | a33 |
| | | | | | | K221 | | | a203 | a203 | K221 | K221 |
| | | | | | | | | | a393 | a393 | a142 | a142 |
| | | | | | | | | | a452 | a452 | a358 | a358 |
| | | | | | | | | | a142 | a142 | a203 | a203 |
| | | | | | | | | | a358 | a358 | a393 | a393 |
| | | | | | | | | | | | a452 | a452 |

### Example 6

### Determination of Optimal Antigen Concentration

Antigen Preparation

Parathryroid hormone (PTH) was biotinylated using Pierce EZ-Link Sulpho-NHS biotin according the manufacturer's directions (Pierce EZ-link Sulpho-NHS Biotin, (Pierce Chemical Co., Rockford, Ill., Catalogue number 21217). When the antigen could not be biotinylated, Costar UV plates were substituted. The use of Costar UV plates increased the time of the assay and generally required the use of considerably more antigen.

Checkerboard ELISA

An assay laid out in a “checkerboard” arrangement was carried out as described below to determine optimal coating concentration of the antigen. The assay was performed using streptavidin-coated 96-well plates (Sigma SA mitcrotiter plates, Sigma-Aldrich Chemicals, St Louis Mo., Catalogue number-M5432) as follows.

The parathyroid hormone (PTH) antigen was biotinylated using Pierce EZ-link Sulpho-NHS biotin ((Pierce Chemical Co, Rockford Ill., catalog number 21217) according to manufacturer's instructions. Biotinylated antigen diluted in 1% skim milk/1×PBS pH 7.4 in a series of stepwise dilutions from a beginning concentration of 500 ng/ml to a final concentration of 0.5ng/ml. Diluted biotinylated antigen was distributed horizontally across a 96-well Sigma SA microtiter plate (Sigma Aldrich Chemicals, catalogue M-5432), placing 50 ul of each dilution in wells of each of columns 1 through 11, with replicates in each well of rows A-H under each column. No antigen was added to column 12. The plate was incubated at room temperature for 30 minutes. No blocking step was performed because Sigma SA plates are pre-blocked.

The plate was washed four times with tap water. Plates were washed by hand, or using a microplate washer when available.

An anti-PTH antibody with known affinity was used as a reference antibody. Anti-PTH antibody 15g2 was diluted 1% skim milk/1×PBS pH 7.4/0.05% to final initial dilution of 1 ug/ml was serially diluted 1:2, 7 wells to an ending concentration 15 ng/ml and 50 ul of each dilution was distributed in each well of row A to row G, with replicates in each well of columns 1-12. No antibody was added to row H. Plates containing the antigen and reference antibody were incubated at room temperature for approximately 24 hours.

The plate was wrapped tightly (“air tight”) with plastic wrap or paraffin film, and incubated overnight with shaking using a Lab Line Titer Plate Shaker at setting 3.

The plates were washed five times (5×) with water to remove unbound reference antibody. Bound reference antibody was detected by adding fifty microliters (50 ul) of 0.5 ug/ml goat anti-Human IgG Fc HRP polyclonal antibody (Pierce Chemical Co, Rockford Ill., catalog number 31416) in 1% skim milk/1×PBS pH 7.4 to each well and incubating the plate 1 hr at room temperature. (Gt anti-Human Fc HRP—Pierce catalogue number-31416).

The plate was washed at least five times (5×) with water to remove unbound goat anti-Human IgG Fc HRP polyclonal antibody

Fifty microliters (50 ul) of the HRP substrate TMB (Kirkegaard & Perry Laboratories, Inc, Gaithersberg, Md.) was added to each well and the plate was incubated for one-half hour at room temperature. The HRP-TMB reaction was stopped by adding 50 ul of 1M phosphoric acid to each well. Optical density (absorbance) at 450 nm was measured for each well of the plate.

Data Analysis

Table 2 shows the results from the reference assay using PTH as the antigen and 15g2 anti-PTH as the reference antibody. OD measurements from the row of samples corresponding to the reference antibody concentration of 100 ng/ml were examined to find the antigen concentration that gives an OD of approximately 1.0. This concentration was determined to be approximately 15 ng/ml PTH. This concentration of antigen was considered the optimal antigen concentration and will be used for the subsequent experiments.

TABLE 5
Optical Density Measurements of Test Antibodies Bound to
Various Concentrations of PTH

PTH Contration (ng/mL)

| | | 500.00 | 250.00 | 125.00 | 62.50 | 31.25 | 15.63 | 7.81 | 3.91 | 1.95 | 0.98 | 0.49 | 0.00 |
|---|---|---|---|---|---|---|---|---|---|---|---|---|---|
| Reference | 1000 | 3.218 | 3.273 | 3.075 | 3.103 | 2.521 | 1.910 | 1.269 | 0.885 | 0.438 | 0.329 | 0.256 | 0.086 |
| antibody | 500 | 3.199 | 3.133 | 3.144 | 3.068 | 2.608 | 1.928 | 1.283 | 0.708 | 0.424 | 0.293 | 0.224 | 0.062 |
| concentra- | 250 | 3.130 | 3.274 | 3.208 | 2.945 | 2.393 | 1.634 | 3.182 | 0.543 | 0.295 | 0.201 | 0.156 | 0.055 |
| tion (ng/mL) | 125 | 3.190 | 3.194 | 3.177 | 2.733 | 2.116 | 1.251 | 0.863 | 0.444 | 0.489 | 0.178 | 0.147 | 0.067 |
| | 62.5 | 3.187 | 3.262 | 2.952 | 2.137 | 1.678 | 0.946 | 0.515 | 0.295 | 0.179 | 0.126 | 0.103 | 0.055 |
| | 31.3 | 3.148 | 3.001 | 2.628 | 1.767 | 1.168 | 0.604 | 0.336 | 0.199 | 0.131 | 0.098 | 0.127 | 0.063 |
| | 15.6 | 2.998 | 2.792 | 2.099 | 1.245 | 0.736 | 0.371 | 0.189 | 0.127 | 0.093 | 0.073 | 0.070 | 0.056 |
| | 0 | 0.114 | 0.121 | 0.089 | 0.088 | 0.069 | 0.068 | 0.054 | 0.052 | 0.054 | 0.057 | 0.058 | 0.063 |

### Example 7

### Limiting Antigen Assay of Test Antibodies

SA microtiter plates were coated with biotinylated antigen PTH at the optimal concentration of 15 ng/ml as determined in Example 6. Fifty microliters (50 ul) of biotinylated antigen at a concentration of 15 ng/ml in 1% skim milk/1×PBS pH 7.4 was added to each well, in a dilution pattern as described in Example 1. The plate was incubated for 30 minutes.

Plates were washed four times (4×) with water, and a B-cell culture supernatant containing test antibodies diluted 1:10 in 1% skim milk/1×PBS pH 7.4/0.05% azide, and 50 ul of each sample was added to each of two wells. Forty-eight (48) different samples were added per 96 well plate. On a separate plate, reference antibody 15g2 anti-PTH at the concentration used to determine the optimal antigen concentration was diluted out at least half a plate. This provided a positive control to assure that results from assays of test antibodies are comparable with optimization results.

Plates were wrapped tightly with plastic wrap or paraffin film, and incubated with shaking for 24 hours at room temperature.

On the following day, all plates were washed five times (5×) and 50 ul goat anti-Human IgG Fc HRP polyclonal antibody at a concentration of 0.5 ug/ml in 1% milk/1×PBS pH 7.4 was added to each well. The plates were incubated for 1 hour at room temperature.

Plates were washed at least five times (5× with tap water). Fifty microliters (50) ul of HPR substrate TMB was added to each well, and the plate were incubated for 30 minutes. The HRP-TMB reaction was stopped by adding 50 ul of 1M phosphoric acid to each well. Optical density (absorbance) at 450 nm was measured for each well of the plate.

Data Analysis

OD values of test antibodies were averaged and the range was calculated. Antibodies with the highest signal and acceptably low standard deviation were selected as antibodies having a higher affinity for the antigen than did the reference antibody.

Table 6 shows the results of a limiting antigen dilution assay using PTH as a ligand. Antibodies are ranked according to their relative affinity for various PTH antigens, and identified by their well number.

TABLE 6
Affinity Ranking of Test Antibodies to Limited Dilution of PTH

| | Limiting | Limiting | Primary | Secondary | | | | Rat |
|---|---|---|---|---|---|---|---|---|
| Well | Ag OD | Ag Rank | OD | OD | PTH(1-84) | PTH(7-84) | PTH(17-44) | PTH(1-84) |
| 292A10 | 2.747 | 1 | 0.992 | ND | 1.40 | 1.95 | 3.26 | 0.62 |
| 302A7 | 1.376 | 2 | 0.317 | ND | 0.35 | 0.36 | 2.66 | 0.19 |
| 253D10 | 1.009 | 3 | 0.954 | 0.511 | 0.79 | 1.10 | 2.10 | 1.18 |
| 263C8 | 0.693 | 5 | 0.372 | 0.286 | 1.75 | 1.98 | 3.29 | 1.34 |
| 245B10 | 0.644 | 6 | 0.622 | 0.580 | 0.84 | 0.32 | 0.12 | 0.19 |
| 238F8 | 0.566 | 7 | 0.667 | 0.541 | 1.05 | 1.34 | 2.79 | 1.19 |
| 228E3 | 0.504 | 8 | 0.560 | 0.259 | 0.48 | 0.80 | 3.12 | 1.40 |
| 262H1 | 0.419 | 9 | 0.461 | 0.274 | 0.86 | 1.20 | 2.45 | 0.36 |
| 161G7 | 0.411 | 10 | 0.409 | 0.212 | 0.49 | 0.90 | 1.88 | 0.84 |
| 331H6 | 0.322 | 11 | 0.312 | ND | 0.52 | 0.45 | 2.40 | 0.24 |
| 287E7 | 0.261 | 12 | 0.682 | ND | 0.71 | 0.13 | 0.36 | 1.03 |
| 315D8 | 0.221 | 13 | 0.441 | ND | 0.14 | 0.17 | 0.29 | 0.31 |
| 279E6 | 0.213 | 14 | 0.379 | ND | 0.31 | 0.10 | 0.17 | 0.19 |
| 250G6 | 0.178 | 15 | 0.560 | 0.248 | 0.44 | 0.66 | 1.77 | 0.19 |
| 244H11 | 0.175 | 16 | 0.405 | 0.556 | 0.50 | 0.86 | 0.98 | 0.31 |
| 313D5 | 0.170 | 17 | 0.664 | ND | 0.12 | 0.29 | 0.43 | 0.30 |
| 339F5 | 0.120 | 18 | 0.319 | ND | 0.40 | 0.21 | 0.11 | 0.25 |
| 279D2 | 0.114 | 19 | 0.353 | ND | 0.31 | 0.11 | 0.27 | 0.18 |
| 307H1 | 0.084 | 20 | 0.401 | ND | 0.10 | 0.14 | 0.30 | 0.42 |
| 308A1 | 0.079 | 21 | 0.312 | ND | 0.19 | 0.22 | 0.30 | 0.45 |
| 322F2 | ND | 22 | 1.870 | ND | 1.01 | 0.15 | 0.34 | 1.41 |

### Example 8

### Dilutions of Antibodies Against Interleukin-8 (IL-8)

The proper coating concentration of IL-8 was determined as described above to determine a concentration of IL-8 that resulted in an OD of approximately 1. The optimal concentration was then incubated with a variety of anti-IL-8 antibody supernatants derived from XenoMouse animals immunized with IL-8. Table 4 illustrates typical results and ranking of antibodies screened for their affinity for IL-8. The columns “primary OD” and “secondary OD” refer to primary and secondary binding screen OD's achieved when non-limited amounts of IL-8 were used in the binding ELISA. OD values reported in the limited antigen section refer to an average of two binding ELISA's done at limited antigen. As shown by Table 7, the top three atibodies are able retain their binding to antigen even at the limited concentrations. Other antibodies which also achieved high OD's in the primary and secondary non-limited antigen binding ELISA were not able to achieve the same signal when antigen concentrations were limiting.

TABLE 7
Affinity Ranking of Test Antibodies to Limited Dilution of IL-8

| | | | | Limited Ag | | |
|---|---|---|---|---|---|---|
| Clone Number | | Primary | Secondary | | | Limited |
| plate | well | OD | OD | Average | St dev. | Ag Rank |
| 36 | C6 | 1.95 | 3.023 | 1.32 | 4% | 1 |
| 6 | G11 | 2.021 | 1.403 | 0.90 | 9% | 2 |
| 50 | B1 | 1.818 | 2.398 | 0.82 | 14% | 3 |
| 41 | C11 | 1.83 | 3.218 | 0.81 | 19% | 4 |
| 53 | G5 | 1.128 | 2.521 | 0.80 | 1% | 5 |
| 44 | B8 | 2.09 | 2.707 | 0.78 | 2% | 6 |
| 51 | G10 | 1.408 | 1.652 | 0.78 | 2% | 7 |
| 53 | E1 | 1.992 | 3.035 | 0.72 | 12% | 8 |
| 38 | C1 | 2.571 | 2.945 | 0.71 | 3% | 9 |
| 32 | F3 | 2.339 | 3.322 | 0.66 | 13% | 10 |
| 13 | F10 | 1.505 | 1.833 | 0.66 | 5% | 11 |
| 41 | D2 | 2.997 | 2.944 | 0.66 | 5% | 12 |
| 53 | C2 | 1.56 | 1.869 | 0.64 | 22% | 13 |
| 14 | E2 | 1.255 | 1.875 | 0.57 | 25% | 14 |
| 54 | C3 | 2.131 | 2.486 | 0.51 | 12% | 15 |
| 50 | F3 | 0.572 | 1.635 | 0.51 | 26% | 16 |
| 55 | E8 | 1.031 | 1.917 | 0.50 | 10% | 17 |
| 42 | E5 | 3.07 | 3.147 | 0.49 | 4% | 18 |
| 6 | E7 | 0.637 | 1.545 | 0.49 | 22% | 19 |
| 7 | E10 | 1.794 | 1.953 | 0.48 | 18% | 20 |
| 8 | B2 | 1.725 | 1.777 | 0.48 | 5% | 21 |
| 48 | E6 | 2.103 | 3.004 | 0.48 | 25% | 22 |
| 33 | A1 | 2.623 | 2.351 | 0.47 | 17% | 23 |
| 51 | F5 | 2.062 | 2.838 | 0.45 | 15% | 24 |
| 51 | B1 | 1.778 | 2.631 | 0.45 | 0% | 25 |
| 44 | A5 | 2.473 | 2.55 | 0.44 | 5% | 26 |
| 6 | G4 | 2.117 | 1.505 | 0.41 | 7% | 27 |
| 43 | G4 | 0.991 | 1.943 | 0.41 | 2% | 28 |
| 47 | E3 | 1.049 | 2.222 | 0.40 | 16% | 29 |
| 46 | F11 | 1.641 | 1.843 | 0.39 | 9% | 30 |
| 43 | F4 | 0.744 | 1.449 | 0.39 | 7% | 31 |
| 54 | H1 | 1.465 | 1.584 | 0.38 | 25% | 32 |
| 44 | F4 | 2.05 | 2.573 | 0.38 | 13% | 33 |
| 49 | G11 | 1.334 | 2.019 | 0.37 | 6% | 34 |
| 11 | C10 | 1.169 | 1.498 | 0.37 | 3% | 35 |
| 41 | B12 | 1.107 | 1.347 | 0.37 | 3% | 36 |
| 46 | F2 | 0.865 | 1.15 | 0.37 | 11% | 37 |
| 52 | E11 | 0.961 | 2.034 | 0.37 | 5% | 38 |
| 7 | B6 | 2.039 | 1.802 | 0.33 | 6% | 39 |
| 39 | F6 | 1.434 | 1.196 | 0.33 | 6% | 40 |
| 10 | E5 | 0.886 | 1.262 | 0.33 | 6% | 41 |
| 36 | C12 | 1.078 | 1.991 | 0.33 | 10% | 42 |
| 44 | B9 | 1.469 | 1.683 | 0.32 | 4% | 43 |
| 8 | H1 | 1.338 | 1.316 | 0.31 | 2% | 44 |
| 52 | F3 | 1.289 | 1.204 | 0.28 | 16% | 45 |
| 45 | A4 | 1.136 | 1.302 | 0.28 | 13% | 46 |
| 25 | A11 | 1.199 | 1.17 | 0.27 | 25% | 47 |
| 51 | C12 | 0.955 | 1.148 | 0.26 | 11% | 48 |
| 6 | E5 | 1.41 | 1.138 | 0.24 | 8% | 49 |
| 39 | H3 | 0.471 | 1.155 | 0.23 | 6% | 50 |
| 14 | E3 | 1.958 | 1.255 | 0.22 | 15% | 51 |
| 3 | D1 | 2.254 | 3.497 | 0.21 | 24% | 52 |
| 33 | F4 | 1.323 | 1.408 | 0.21 | 24% | 53 |
| 51 | A12 | 0.555 | 1.522 | 0.19 | 17% | 54 |
| 5 | G1 | 2.205 | 2.274 | 0.17 | 4% | 55 |
| 35 | C9 | 1.217 | 1.249 | 0.17 | 4% | 56 |
| 6 | B10 | 1.006 | 1.145 | 0.17 | 8% | 57 |
| 39 | B4 | 1.326 | 1.62 | 0.17 | 8% | 58 |
| 5 | G3 | 1.192 | 1.387 | 0.17 | 29% | 59 |
| 35 | F10 | 1.307 | 1.777 | 0.17 | 29% | 60 |
| 17 | E11 | 0.839 | 1.805 | 0.17 | 15% | 61 |
| 3 | D3 | 0.605 | 1.351 | 0.16 | 5% | 62 |
| 31 | A1 | 1.557 | 1.826 | 0.16 | 17% | 63 |
| 28 | C5 | 1.373 | 1.942 | 0.16 | 5% | 64 |
| 14 | F5 | 1.441 | 1.482 | 0.15 | 25% | 65 |
| 43 | D8 | 0.714 | 1.501 | 0.15 | 22% | 66 |
| 29 | D5 | 1.326 | 1.322 | 0.14 | 23% | 67 |
| 32 | F11 | 1.36 | 1.284 | 0.48 | 71% | 68 |
| 7 | D4 | 0.874 | 2.333 | 0.44 | 34% | 69 |
| 47 | G11 | 0.811 | 1.209 | 0.42 | 76% | 70 |
| 39 | G2 | 0.676 | 1.157 | 0.42 | 32% | 71 |
| 15 | G4 | 2.046 | 2.461 | 0.39 | 41% | 72 |
| 31 | G12 | 1.902 | 1.929 | 0.36 | 44% | 73 |
| 41 | C2 | 1.201 | 2.522 | 0.33 | 34% | 74 |
| 7 | E11 | 1.402 | 1.719 | 0.32 | 50% | 75 |
| 40 | A4 | 1.786 | 1.427 | 0.32 | 50% | 76 |
| 45 | E12 | 1.986 | 2.887 | 0.26 | 54% | 77 |
| 2 | B10 | 1.871 | 1.389 | 0.22 | 38% | 78 |
| 7 | H8 | 1.516 | 1.171 | 0.22 | 45% | 79 |
| 28 | C3 | 1.246 | 1.182 | 0.15 | 52% | 80 |

TABLE 7A
Affinity Measurement of Reference Antibody 1
Reference antibody 1

| Conc. ng/ml | Limited Ag OD | St. Dev. |
|---|---|---|
| 125.00 | 1.52 | 1% |
| 62.50 | 1.38 | 2% |
| 31.25 | 1.25 | 12% |
| 15.63 | 1.13 | 28% |
| 7.81 | 0.80 | 2% |
| 3.91 | 0.78 | 18% |
| 1.95 | 0.67 | 0% |
| 0.98 | 0.73 | 8% |
| 0.49 | 0.53 | 18% |
| 0.24 | 0.39 | 17% |

TABLE 7B
Affinity Measurement of Reference Antibody 2
Reference antibody 2

| Conc. ng/ml | Limited Ag OD | St. Dev. |
|---|---|---|
| 125.00 | 0.52 | 23% |
| 62.50 | 0.38 | 11% |
| 31.25 | 0.34 | 1% |
| 15.63 | 0.42 | 43% |
| 7.81 | 0.54 | 13% |
| 3.91 | 0.46 | 30% |
| 1.95 | 0.54 | 9% |
| 0.98 | 0.34 | 9% |
| 0.49 | 0.49 | 32% |
| 0.24 | 0.55 | 38% |

### Example 9

### Affinity Ranking

Preparation of Antigens

In order to increase the effective throughput of the antibody affinity ranking process, we labeled different concentrations of an antigen with different colored beads. In this example, beads from the Luminex system were used. As is known, each bead, when activated, emits light of a varying wavelength. When put in a Luminex reader, the identity of each bead can be readily ascertained.

In this example, a different color of strepavidin luminex bead was bound to each of four concentrations of biotinylated antigen (1 ug/ml, 100 ng/ml, 30 ng/ml, and 10 ng/ml). Thus, each concentration of the antigen was represented by a different color bead. The four concentrations were the mixed into a single solution containing all four color-bound concentrations.

All of the antibody samples were then diluted to the same concentration (˜500 ng/ml) using Luminex quantitation results or a one-point quantitation by Luminex. A serial dilution (1:5) of all of the samples was then performed so a total of four dilution points were obtained, while preferably diluting enough sample for two plates: a quantitation plate and the ranking plate.

Ranking of Antibodies

In order to rank the antibodies, ˜2000 of each mixture of luminex bead-antigen samples was loaded into each well of the luminex plate, and then the well was aspirated. Then 50 ul of each antibody sample (24 samples total) was loaded into each well and left overnight while shaking in 4° C. The plates were washed three times (3×) with washing buffer. Detection with a fluorescent anti-human antibody (hIgG-Phycoerythrin (PE) (1:500 dilution)) that bound 50 ul/well was then performed while shaking at room temperature for 20 min. The plates were then washed three times (3×) with washing buffer. The plates were re-suspended in 80 ul blocking buffer. Next, the plates were loaded in the Luminex apparatus.

Data Analysis

Because each well held four different concentrations of the same antigen, that could be distinguished based on color, it was possible to rapidly rank binding affinities of the different antibodies. For example, antibodies that had very strong binding affinity for the antigen bound to even the weakest dilution of antibody. This could be measured by analyzing the amount of fluorescent anti-human antibody bound to the colored bead attached to the weakest antigen concentration. Alternatively, antibodies that did not bind strongly might were only detected as binding with the 1 ug/ml and 100 ng/ml antigen concentrations, but not the 30 ng/ml or 10 ng/ml concentrations.

Data analysis was performed using SoftMax Pro for the quantitation data. The Luminex signal of samples tested at several concentrations were compared. The samples were then ranked accordingly.

### Example 10

### Comparison of Limiting Antigen Output Compared to Absolute Biacore KD Measurements

The following kinetic ranking technique was performed by ELISA and compared to formal BiaCore kinetics. Below in Table 8 is a comparison of a typical limited antigen output as compared to absolute Biacore derived KD measurements. In short, 68 antibodies were ranked (relative to each other) using limited antigen ranked. From the 68 antibodies 17 were scaled up to sufficient quantities for formal affinity measurements using BiaCore technology.

TABLE 8
Comparison of Affinity Measurement Based on Limited
Dilutions with Biacore Affinity Measurements

| | Limited | |
|---|---|---|
| | Antigen | Biacore |
| Sample ID | Ranking | Affinity (nM) |
| A | 1 | 1.9 |
| B | 3 | 1.9 |
| C | 4 | 1.3 |
| D | 5 | 6.9 |
| E | 7 | 3.3 |
| F | 10 | 17.7 |
| G | 11 | 28.9 |
| H | 12 | 3.8 |
| I | 13 | 4.4 |
| J | 23 | 11.2 |
| K | 28 | 57.8 |
| L | 30 | 29.2 |
| M | 34 | 1667 |
| N | 46 | 115.2 |
| O | 47 | 305.1 |
| P | 51 | 1000 |
| Q | 60 | 33.1 |

Data Analysis

As can be seen overall there is a high degree of correlation between high limited antigen rank and the formal KD. In the case of antibodies which do not correlate well, there are a number of reasons why such discrepancies could exist. For example, although antigen is coated on ELISA plates at a low density avidity effects cannot completely be ruled out. In addition, it is possible that, when coating assay material for the limited antigen ranking technique, certain epitopes could be masked or altered. In Biacore analysis, if antigen is flowed over an antibody coated chip, these epitopes on the antigen could be presented in a different conformation and, therefore, seen at a different relative concentration. This could, in turn, could result in a different kinetic ranking between the two methods.

It is also possible that an antibody with lower Biacore derived affinities may give a high limited antigen rank due to a much higher than average concentration of antigen specific antibody being present in the test sample. This could, in turn, lead to an artificially high limited antigen score.

Importantly, the limited antigen kinetics method did allow a rapid determination of relative affinity and it identified the antibodies with the highest formal affinity of the tested antibodies in this panel. Further, as the limited antigen kinetic relative ranking method is easily scalable to interrogate thousands of antibodies at early stages of antibody generation it offers significant advantage over other technologies which do not offer similar advantages of scale.

It will be understood by those of skill in the art that numerous and various modifications can be made without departing from the spirit of the present invention. Therefore, it should be clearly understood that the forms of the present invention are illustrative only and are not intended to limit the scope of the present invention.

What is claimed is:

1. A method of identifying potential therapeutic products comprising:
providing a protein target;
identifying molecules that interact with said protein target;
categorizing said molecules that interact with said protein target according to selected criteria;
determining the characteristics of molecules from each said category;
identifying characteristics of said molecules from each said category that indicate potential therapeutic utility of said protein target; and
determining the potential therapeutic utility of said protein target in connection with said molecules that interact with said protein target in a way that enables such therapeutic utility.

2. The method of claim 1, wherein said identifying molecules that interact with said protein target comprises screening said protein target against a plurality of molecules.

3. The method of claim 1, wherein said molecules that interact with said protein target are small molecules, protein, peptides, or antibodies.

4. The method of claim 1, wherein said molecules that interact with said protein target are antibodies

5. The method of claim 1, wherein said target protein has a known function or utility.

6. The method of claim 1, wherein said target protein has an unknown function or utility.

7. The method of claim 1, wherein said target protein is an antigen and said molecules that interact with said protein target are antibodies against said antigen.

8. The method of claim 7, wherein said categorizing said molecules that interact with said protein target according to selected criteria comprises categorizing a panel of antibodies according to the epitope on said antigen recognized by said antibodies.

9. The method of claim 8, further wherein said determining the characteristics of said representative molecules from each category comprises determining binding affinity of said panel of antibodies to each said epitope.

10. The method of claim 9, further wherein determining the characteristics of said representative molecules from each category comprises ranking said panel of antibodies according to binding affinity of said antibodies to each said epitope.

11. The method of claim 10, further wherein said identifying characteristics of said representative molecules that indicate potential therapeutic utility of said protein target comprises identifying optimized binding affinity of said panel of antibodies to each said epitope.

12. The method of claim 10 comprising utilizing epitope binning to categorize said panel of antibodies according to the epitope recognized by each said antibody and utilizing at least one limiting antigen dilution assay to kinetically rank said panel of antibodies according to binding affinity of said antibodies to each said epitope.

13. The method of claim 12, comprising utilizing a competitive antibody assay to discern the epitope recognition properties of said panel of antibodies, further comprising utilizing a clustering process to categorize said antibodies in said panel, and further comprising utilizing a limiting antigen dilution assay to kinetically rank said panel of antibodies according to binding affinity of said antibodies to each said epitope.

14. A method for determining the therapeutic potential of an antibody identified by epitope binning and limiting antigen dilution assay as a high-affinity antibody against an antigen of interest comprising evaluating said antibody for the ability to act directly on cells to cause a desired effect.

15. The method of claim 14, wherein said antibody is conjugated, such that said conjugated antibody is evaluated for said ability to act directly on cells to cause a desired effect.

16. The method of claim 15, wherein said conjugated antibody is an immunotoxin.

17. The method of claim 14, comprising determining the therapeutic potential of said antibody to treat a disorder or disease state in an animal.

18. The method of claim 17, wherein said animal is a mammal.

19. The method of claim 18, wherein said mammal is a human.

20. The method of claim 17, wherein said antibody is an antibody against disease-specific antigens.

21. The method of claim 20 wherein said disease-specific antigens are cancer antigens and said disorder or disease state is cancer.

22. The method of claim 21, wherein said cancer comprises solid tumors.
